(19) United States
(12) Patent Application Publication
(10) Pub. No.: US 2023/0332126 A1
(43) Pub. Date: Oct. 19, 2023

(54) Botulinum Neurotoxin Proteins and Methods to Engineer and Generate Same

(71) Applicants: The Regents of the University of California, Oakland, CA (US); Allergan, Inc., Irvine, CA (US)

(72) Inventors: Gregory A. Weiss, Irvine, CA (US); Amy D. Brideau-Andersen, San Clemente, CA (US); Rebekah P. Dyer, Irvine, CA (US); Lance E. Steward, Irvine, CA (US); Linh Q. Le, Tustin, CA (US); Gaetano Speciale, Irvine, CA (US); David C. Rupp, San Pedro, CA (US); Birgitte P.S. Jacky, Orange, CA (US); Madison H. Fletcher, Irvine, CA (US); Sudipta Majumdar, Irvine, CA (US)

(21) Appl. No.: 18/042,244

(22) PCT Filed: Aug. 24, 2021
(86) PCT No.: PCT/US2021/047349

§ 371 Date: Feb. 20, 2023

Related U.S. Application Data
(60) Provisional application No. 63/070,399, filed on Aug. 26, 2020.

(51) Int. Cl. C12N 9/52; C12N 15/10

(52) U.S. Cl. CPC C12N 9/52; C12Y 304/24069; C12N 15/1058

(57) ABSTRACT

Botulinum neurotoxin proteins and fragments thereof that bind and/or cleave a noncanonical substrate, e.g., non-neuronal SNARE proteins such as human SNAP-23 (hSNAP-23) and SNAP-29 (hSNAP-29), are described.

### CROSS-REFERENCE TO RELATED APPLICATIONS

This application claims the benefit of U.S. Patent Application No. 63/070,399, filed Aug. 26, 2020, which is herein incorporated by reference in its entirety.

### STATEMENT REGARDING GOVERNMENT INTEREST

This invention was made with Government support under Grant No. 5T32CA009054-37 awarded by the National Institutes of Health (NIH). The Government has certain rights in the invention.

### REFERENCE TO A SEQUENCE LISTING SUBMITTED VIA EFS-WEB

The content of the ASCII text file of the sequence listing named “20200826_034044_214P1_ST25” which is 124 kb in size was created on Aug. 26, 2020 and electronically submitted via EFS-Web herewith the application is incorporated herein by reference in its entirety.

### TECHNICAL FIELD

The technical field generally relates to botulinum neurotoxin proteases having the ability to cleave non-neuronal SNARE proteins, and the use thereof for suppressing undesirable secretion from a mammalian cell by cleavage of said non-neuronal SNARE proteins in said mammalian cell.

### BACKGROUND

Toxins fall into one of two classes, namely cytotoxic toxins (e.g., plant toxin such as ricin) which kill their natural target cells, and non-cytotoxic toxins (e.g., botulinum neurotoxins) which do not kill their natural target cells. Non-cytotoxic toxins exert their effects on a target cell by inhibiting a cellular process other than protein synthesis.

Botulinum neurotoxin proteases act by proteolytically cleaving intracellular transport proteins known as SNARE proteins (e.g., SNAP-25, VAMP, or Syntaxin). The acronym SNARE derives from the term Soluble NSF Attachment protein Receptor, where NSF means N-ethylmaleimide-Sensitive Factor. SNARE proteins are a large super family of proteins. A function of SNARE proteins is to mediate the exocytosis of neurotransmitter molecules to the post-synaptic junction. SNARE proteins are therefore integral to secretion of molecules via vesicle transport from a cell.

Clostridium botulinum produces different neurotoxins (BoNTs) that are differentiated serologically by the lack of anti-serum cross serotype neutralization. BoNTs elicit neuronal-specific flaccid paralysis by targeting neurons and cleaving neuron-specific SNARE proteins.

BoNTs have a 150 kDa polypeptide chain comprising a 100 kDa heavy chain and a 50 kDa light chain linked by a disulfide bond. BoNTs are organized into three functional domains: an N-terminal proteolytic light chain (L-chain); and a C-terminal heavy chain (H-chain), the latter consisting of a translocation domain (HN) and a C-terminal neuron-binding domain (He).

BoNTs follow a three-step mechanism of action. First, the H_{c} portion binds to a cholinergic nerve cell and becomes internalised via receptor-mediated endocytosis. Secondly, the HN portion translocates the L-chain across the endosomal membrane and into the cytosol of the nerve cell. Thirdly, the L-chain binds to and cleaves a neuronal SNARE protein within the cytosol, thereby suppressing neurotransmitter release from the nerve cell and resulting in nerve cell intoxication.

Native BoNTs are able to target and cleave neuronal SNARE isoforms such as VAMP-1, VAMP-2, VAMP-3, SNAP-25, syntaxin 1a and syntaxin 1b. The protease of BoNT/X, a botulinum neurotoxin identified by bioinformatic approaches, cleaves VAMP-1, VAMP-2, VAMP-3, VAMP-4, VAMP-5 and Ykt6. The BoNT proteases, however, have little or no cleavage effect on the majority of non-neuronal SNARE proteins. The seven classical BoNT serotypes cleave specific residues on one or more SNARE proteins. For example, serotypes B, D, F, and G cleave VAMP-1, VAMP-2 and VAMP-3; serotypes A and E cleave SNAP-25; and serotype C cleaves SNAP-25 and syntaxin 1a. The protease domain from BoNT/En, identified from gram-positive enterococcus, cleaves VAMP-2 and SNAP25. This neuronal SNARE substrate specificity is consistent with and understood to be reflective of the natural neuronal cell binding specificity demonstrated by BoNTs. For example, BoNT/A cleaves human SNAP-25, but not human non-neuronal isoforms.

BoNT/A is known for the treatment of strabismus, blepharospasm, hemifacial spasm, axially hyperhidrosis, and cervical dystonia, and is also used in cosmetic treatment of glabellar facial lines, lateral canthal line, and forehead lines. BoNT/A efficacy in dystonia and other disorders related to involuntary skeletal muscle activity, coupled with a satisfactory safety profile, has prompted empirical/off-label use in a variety of secretions and pain and cosmetic disorders.

Clinical applications of BoNTs have focused on targeting disorders associated with neuromuscular activity. More recently, the design of re-targeted BoNTs that bind to unique subset of neurons (e.g., nociceptive afferents—see WO96/33273, which is hereby incorporated in its entirety) and/or to non-neuronal cells (e.g., airway epithelium cells—see WO00/10598, which is hereby incorporated in its entirety) has been described. This technology involves replacement of the native BoNT binding domain by a different targeting moiety (e.g., a growth factor or other signaling molecule). However, the specific cleavage of neuronal specific SNARE proteins by BoNTs has limited development of new proteins and new therapies. Neuronal and non-neuronal SNARE proteins are believed to be of equal importance to the process of intracellular vesicle fusion, and thus to the secretion of molecules via vesicle transport from a cell. Accordingly, the use of BoNT-based therapeutics to inactivate neuronal SNARE protein driven secretion may not address any corresponding non-neuronal SNARE driven cellular secretion.

For example, the non-neuronal SNARE protein SNAP23 has similar amino acid sequence and function to that of the neuronal SNARE protein SNAP25, but participates in a much greater diversity of vesicle fusion events across many tissues. SNAP23 is also involved in diseases including mucin hypersecretion in asthma and chronic obstructive pulmonary disease, ovarian cancer and malignancy, and granule secretion in inflammatory diseases. BoNT-based therapeutics and BoNT L-chain proteases capable of cleaving the non-neuronal SNARE protein SNAP23 with improved efficiency and/or specificity for treating non-neuronal SNAP23-associated diseases and conditions (e.g., asthma, chronic obstructive pulmonary disease, cancer including ovarian cancer, malignancy, etc.) are desired.

Another SNARE protein, SNAP29, facilitates a cellular recycling process known as autophagy. Inhibiting autophagy could enhance the effects of chemotherapy for cancers. Depletion of SNAP29 severely impedes a fusion event that enables cellular recycling. A BoNT retargeted to cleave SNAP29 would therefore serve as a potent and specific inhibitor of autophagy. SNAP29 has low homology to SNAP25, but shares sequence similarity in a region of SNAP25 and is cleaved by BoNT/E L-chain protease, a BoNT serotype related to BoNT/A L-chain protease. Therefore, retargeting the BoNT/E L-chain protease to cleave SNAP29 for treating SNAP29-associated diseases and conditions (e.g., cancer) is desired.

Accordingly, described is an engineered BoNT L-chain protease that cleaves a non-neuronal SNARE protein with improved efficiency and/or specificity. The engineered or modified BoNT L-chain proteases, including BoNT/A and BoNT/E L-chain proteases, cleave SNARE protein isoforms that are mainly expressed in non-neuronal cells, namely human SNAP-23 (hSNAP-23) and SNAP-29 (hSNAP-29). The engineered botulinum neurotoxin proteins, also referred to as modified botulinum neurotoxin proteins, provide a new class of non-cytotoxic therapeutic agents that may be useful for treating diseases and conditions associated with SNARE proteins, such as non-neuronal SNARE proteins SNAP23 and SNAP29.

### BRIEF SUMMARY

The following aspects and embodiments thereof described and illustrated below are meant to be exemplary and illustrative, not limiting in scope.

In some embodiments, a botulinum neurotoxin protein or fragment thereof is provided that comprises an amino acid sequence having at least about 90%, e.g., about 90%, about 91%, about 92%, about 93%, about 94%, about 95%, about 96%, about 97%, about 98%, or about 99%, sequence identity to SEQ ID NO: 1 and two or more amino acid substitutions selected from the group consisting of: N26X1, wherein X1 is S, T, M, or C; A27X2, wherein X2 is L, R, I, V, M, K, or Q; Q29X3, wherein X3 is R, S, K, M, I, or T; N53X4, wherein X4 is H, R, Q, K, M, or I; E55X5, wherein X5 is I, N, V, L, M, Q, H, or D; E56X6, wherein X6 is I, L, V, or M; Q162X7, wherein X7 is R, K, M, or I; E201X8, wherein X8 is D, N, or Q; D203X9, wherein X9 is V, I, L, or M; N240X10, wherein X10 is A, S, G, T, M, or C; 5254X11, wherein X11 is A, L, M, I, V, G, or C; K364X12, wherein X12 is R, Q, M, or I; and Y387X13, wherein X13 is N, Q, H, E, or D. In some embodiments, the botulinum neurotoxin protein or fragment further comprises one or more additional amino acid substitutions selected from the group consisting of: E148X14, wherein X14 is Y, W, F, or H; K166X15, wherein X15 is F, M, L, Y, W, or H; and G305X16, wherein X16 is G, D, E, N, or Q. In some embodiments, X1 of N26X1 is S. In some embodiments, X2 of A27X2 is L or R. In some embodiments, X3 of Q29X3 is R or S. In some embodiments, X4 of N53X4 is H or R. In some embodiments, X5 of E55X5 is I, N, or V. In some embodiments, X6 of E56X6 is I. In some embodiments, X7 of Q162X7 is R. In some embodiments, X8 of E201X8 is D. In some embodiments, X9 of D203X9 is V. In some embodiments, X10 of N240X10 is A or S. In some embodiments, X11 of 5254X11 is A, L, or M. In some embodiments, X12 of K364X12 is R. In some embodiments, X13 of Y387X13 is N. In some embodiments, X14 of E148X14 is Y. In some embodiments, X15 of K166X15 is F. In some embodiments, X16 of G305X16 is G or D.

In some embodiments, a botulinum neurotoxin protein or fragment thereof is provided that comprises an amino acid sequence having at least about 90%, e.g., about 90%, about 91%, about 92%, about 93%, about 94%, about 95%, about 96%, about 97%, about 98%, or about 99%, sequence identity to SEQ ID NO: 1 and (a) at least amino acid substitution selected from the group consisting of: N26X1, wherein X1 is S, T, M, or C; A27X2, wherein X2 is L, R, I, V, M, K, or Q; Q29X3, wherein X3 is R, S, K, M, I, or T; N53X4, wherein X4 is H, R, Q, K, M, or I; E55X5, wherein X5 is I, N, V, L, M, Q, H, or D; E56X6, wherein X6 is I, L, V, or M; Q162X7, wherein X7 is R, K, M, or I; E201X8, wherein X8 is D, N, or Q; D203X9, wherein X9 is V, I, L, or M; N240X10, wherein X10 is A, S, G, C, T, or M; S254X11, wherein X11 is A, L, M, I, V, G, or C; K364X12, wherein X12 is R, Q, M, or I; and Y387X13, wherein X13 is N, Q, H, E, or D, and (b) at least one amino acid substitution selected from the group consisting of: E148X14, wherein X14 is Y, W, F, or H; K166X15, wherein X15 is F, M, L, Y, W, or H; and G305X16, wherein X16 is G, D, E, N, or Q, and with the proviso that the amino acid sequence is not SEQ ID NO: 27. In some embodiments, X1 of N26X1 is S. In some embodiments, X2 of A27X2 is L or R. In some embodiments, X3 of Q29X3 is R or S. In some embodiments, X4 of N53X4 is H or R. In some embodiments, X5 of E55X5 is I, N, or V. In some embodiments, X6 of E56X6 is I. In some embodiments, X7 of Q162X7 is R. In some embodiments, X8 of E201X8 is D. In some embodiments, X9 of D203X9 is V. In some embodiments, X10 of N240X10 is A or S. In some embodiments, X11 of 5254X11 is A, L, or M. In some embodiments, X12 of K364X12 is R. In some embodiments, X13 of Y387X13 is N. In some embodiments, X14 of E148X14 is Y. In some embodiments, X15 of K166X15 is F. In some embodiments, X16 of G305X16 is G or D. In some embodiments, the botulinum neurotoxin protein or fragment thereof, comprises an amino acid sequence having at least about 90% sequence identity to SEQ ID NO: 1 and amino acid substitution 5254X11 wherein X11 is L or M and/or amino acid substitution N53H, and optionally further including one or more of the following amino acid substitutions: N26S, Q29R, E55V, E148Y, K166F, N240A, G305D. In some embodiments, the botulinum neurotoxin protein or fragment thereof, comprises an amino acid sequence having at least about 90% sequence identity to SEQ ID NO: 1 and amino acid substitutions E201D and D203V, and optionally further including one or more of the following amino acid substitutions: K166F, N240A, S254A, G305D.

In some embodiments, a botulinum neurotoxin protein is provided that comprises an amino acid sequence with at least about 90% sequence identity to SEQ ID NO: 1 and wherein the amino acid sequence has at least two amino acid positions with an amino acid modification set forth in Table 1 and at least one amino acid position with one of the amino acid modifications set forth in Table 3.

In some embodiments, a botulinum neurotoxin protein is provided that comprises an amino acid sequence with at least about 90% sequence identity to SEQ ID NO: 1 and wherein the amino acid sequence has (i) one set of amino acid modifications set forth in Table 2 and (ii) one or more amino acid positions with an amino acid modification set forth in Table 3.

In some embodiments, a botulinum neurotoxin protein is provided that comprises an amino acid sequence with at least about 90% sequence identity to SEQ ID NO: 1 and wherein the amino acid sequence has one or more amino acid positions with one or more of the amino acid substitutions set forth in Table 3.

In some embodiments, a botulinum neurotoxin protein is provided that comprises an amino acid sequence with at least about 90% sequence identity to SEQ ID NO: 1 and wherein the amino acid sequence has (i) at least two amino acid positions modified by an amino acid modification set forth in Table 1 and (ii) one or more of the amino acid modifications set forth in Table 4 or one set of amino acid modifications set forth in Table 4.

In some embodiments, a botulinum neurotoxin protein is provided that comprises an amino acid sequence with at least about 90% sequence identity to SEQ ID NO: 1 and wherein the amino acid sequence has (i) at least one set of amino acid modifications set forth in Table 2 and (ii) one or more of the amino acid modifications set forth in Table 4 or one set of amino acid modifications set forth in Table 4.

In some embodiments, a botulinum neurotoxin protein is provided that comprises an amino acid sequence with at least about 90% sequence identity to SEQ ID NO: 1 and wherein the amino acid sequence has (i) at least 2 of the following amino acid substitutions (or modifications): E148Y, K166F, S254A, G305D, and (ii) one or more of the amino acid substitutions (or modifications) set forth in Table 3.

In some embodiments, a botulinum neurotoxin protein is provided that comprises an amino acid sequence with at least about 90% sequence identity to SEQ ID NO: 1 and wherein the amino acid sequence has (i) at least 2 of the following amino acid substitutions: E148Y, K166F, S254A, G305D, and (ii) one or more of the amino acid substitutions (modifications) set forth in Table 4 or one set of amino acid substitutions (modifications) set forth in Table 4.

In some embodiments, a botulinum neurotoxin protein is provided that comprises an amino acid sequence with at least about 90% sequence identity to SEQ ID NO: 1 and wherein the amino acid sequence has one set of amino acid substitutions set forth in Table 5.

In some embodiments, a botulinum neurotoxin protein is provided that comprises an amino acid sequence of with at least about 90% sequence identity to SEQ ID NO: 1 and wherein the amino acid sequence has one set of amino acid substitutions set forth in Table 6.

In some embodiments, a botulinum neurotoxin protein is provided that comprises an amino acid sequence with at least about 90% sequence identity to SEQ ID NO: 1 and wherein the amino acid sequence has one of the following amino acid substitutions:
(i) N53H;
(ii) E148Y;
(iii) K166F;
(iv) E148Y and K166F;
(v) S254L; or
(vi) S254M.

In some embodiments, the botulinum neurotoxin protein or fragment cleaves human SNAP23.

In some embodiments, the botulinum neurotoxin protein or fragment thereof is at least about 1.5 fold more specific for SNAP23 than for SNAP25.

In some embodiments, the botulinum neurotoxin protein or fragment thereof is at least about 5 fold more specific for SNAP23 than for SNAP25.

In some embodiments, the botulinum neurotoxin protein or fragment thereof is at least about 10 fold more specific for SNAP23 than for SNAP25.

In some embodiments, the botulinum neurotoxin protein or fragment thereof is at least about 10 fold more specific for cleaving SNAP23 than a botulinum neurotoxin protein with an amino acid sequence having these four modifications: E148Y, K166F, S254A, G305D, with reference to SEQ ID NO: 1.

In some embodiments, the botulinum neurotoxin protein or fragment thereof is at least about 20 fold more specific for cleaving SNAP23 than a botulinum neurotoxin protein with an amino acid sequence having these four modifications: E148Y, K166F, S254A, G305D, with reference to SEQ ID NO: 1.

In some embodiments, the botulinum neurotoxin protein or fragment thereof is at least about 40 fold more specific for cleaving SNAP23 than a botulinum neurotoxin protein with an amino acid sequence having these four modifications: E148Y, K166F, S254A, G305D, with reference to SEQ ID NO: 1.

In some embodiments, the botulinum neurotoxin protein or fragment thereof comprises the amino acid substitution of S254M, with reference to SEQ ID NO: 1.

In some embodiments, the botulinum neurotoxin protein or fragment thereof is at least about 100 fold more specific for cleaving SNAP23 than a botulinum neurotoxin protein with an amino acid sequence having these four modifications: E148Y, K166F, S254A, G305D, with reference to SEQ ID NO: 1.

In some embodiments, the botulinum neurotoxin protein or fragment thereof comprises the amino acid substitution of S254L, with reference to SEQ ID NO: 1.

In some embodiments, the botulinum neurotoxin protein or fragment thereof is at least about 1300 fold more specific for cleaving SNAP23 than a botulinum neurotoxin protein with an amino acid sequence having these four modifications: E148Y, K166F, S254A, G305D, with reference to SEQ ID NO: 1.

In some embodiments, the botulinum neurotoxin protein or fragment thereof with the at least about 1300 fold more SNAP23 cleavage specificity is obtained under physiological salt conditions.

In some embodiments, the physiological salt conditions include 50 mM KH₂PO₄ at pH 7.4.

In some embodiments, the protein or fragment thereof comprises the amino acid substitution of N53H, with reference to SEQ ID NO: 1.

In some embodiments, the botulinum neurotoxin protein or fragment thereof is at least about 120 fold more specific for cleaving SNAP23 than a botulinum neurotoxin protein with an amino acid sequence having these four modifications: E148Y, K166F, S254A, G305D, with reference to SEQ ID NO: 1.

In some embodiments, the botulinum neurotoxin protein or fragment thereof with the at least about 120 fold more SNAP23 cleavage specificity is obtained under physiological salt conditions supplemented with zinc.

In some embodiments, the physiological salt conditions include 50 mM KH₂PO₄ and 0.2 nM ZnCl₂ at pH 7.4.

In some embodiments, the protein or fragment thereof comprises the amino acid substitution of N53H, with reference to SEQ ID NO: 1.

In some embodiments, a botulinum neurotoxin protein or fragment thereof having at least about 80%, 85%, 90%, 95%, 98%, 99% or 99.9% sequence identity to the botulinum neurotoxin protein or fragment of any of the botulinum neurotoxin protein described herein is provided.

In some embodiments, a botulinum neurotoxin protein or fragment thereof comprises (i) a protein having at least about 80%, 85%, 90%, 95%, 98%, 99% or 99.9% sequence identity to the botulinum neurotoxin protein or fragment of any of the botulinum neurotoxin protein or fragments described herein; or (ii) a protein identical to the botulinum neurotoxin protein or fragment as described herein; and a heavy chain protein from a botulinum neurotoxin or fragment thereof.

In some embodiments, the heavy chain protein is a heavy chain protein of botulinum neurotoxin serotype A.

In some embodiments, a botulinum neurotoxin protein or fragment thereof comprises an amino acid sequence of SEQ ID NO: 28.

In some embodiments, a botulinum neurotoxin protein comprises an amino acid sequence with at least about 80%, 85%, 90%, 95%, 98%, 99% or 99.9% sequence identity to SEQ ID NO: 28.

In some embodiments, a botulinum neurotoxin protein or fragment thereof comprises (i) a protein with at least about 80%, 85%, 90%, 95%, 98%, 99% or 99.9% sequence identity to the botulinum neurotoxin protein or fragment thereof SEQ ID NO: 28; or (ii) a protein identical to the botulinum neurotoxin protein or fragment thereof of SEQ ID NO: 28; and a heavy chain protein from a botulinum neurotoxin or fragment thereof.

In some embodiments, the heavy chain protein is a heavy chain protein from botulinum neurotoxin serotype E.

In some embodiments, the botulinum neurotoxin protein or fragment thereof described herein has an improved specificity for a non-canonical substrate relative to its canonical substrate.

In some embodiments, the canonical substrate is SNAP25 and the non-canonical substrate is SNAP23, SNAP29, or a SNAP25/29 chimeric substrate.

In some embodiments, the canonical SNAP25 substrate comprises the amino acid sequence of SEQ ID NO: 25.

In some embodiments, the non-canonical SNAP23 substrate comprises the amino acid sequence of SEQ ID NO: 24.

In some embodiments, the non-canonical SNAP29 substrate comprises the amino acid sequence of SEQ ID NO: 4.

In some embodiments, the non-canonical SNAP25/29 chimeric substrate comprises the amino acid sequence of SEQ ID NO: 29.

In some embodiments, the botulinum neurotoxin protein or fragment thereof is botulinum serotype A, B, C, D, E, F, G, a mosaic neurotoxin, a non-clostridial botulinum toxin-like encoding sequence, or combinations thereof.

In some embodiments, a nucleic acid encoding a botulinum neurotoxin or fragment thereof of the disclosure is provided herein.

In some embodiments, a plasmid is provided that comprises the nucleic acids described herein.

In some embodiments, a vector is provided that comprises the plasmids described herein.

In some embodiments, a host cell is provided that comprises the vectors described herein.

In some embodiments, an expression system is provided that comprises the host cells described herein.

In some embodiments, the expression system is selected from the group consisting of bacteria, yeast (for example Pichia), baculovirus in insect cell, cell-free expression, mammalian cell lines, animals, and phage.

In some embodiments, the expression system is an E. coli expression system.

In some embodiments, a method of generating any one of the botulinum neurotoxin proteins or fragment thereof is provided, the method comprises culturing the host cell described herein under conditions sufficient for the expression of the botulinum neurotoxin protein or fragment thereof, and obtaining the botulinum neurotoxin protein or fragment thereof from the culture.

In some embodiments, a method of modulating substrate specificity of a botulinum neurotoxin protein or fragment thereof is provided, the method comprises adding a ligand to a composition comprising the botulinum neurotoxin protein or fragment thereof.

In some embodiments, the ligand is a cation or a small molecule.

In some embodiments, the cation is a bivalent metal ion.

In some embodiments, the metal ion a zinc ion (Zn²⁺).

In some embodiments, a method for engineering a protease domain of a botulinum neurotoxin or fragment thereof to bind and/or cleave a non-canonical substrate is provided. The method comprises (i) identifying sites in a protease domain of a botulinum neurotoxin or fragment thereof involved in substrate binding and/or catalysis; (ii) constructing a library of protease domain gene mutants of botulinum neurotoxin or fragment thereof for the identified sites; (iii) transforming each gene mutant in the library into an expression system; (iv) expressing protein from clonal populations of each expression system; (v) testing the expressed protein for binding to or cleavage of a non-canonical substrate to identify expressed proteins with improved substrate binding and/or cleavage; (vi) sequencing protein identified to have improved substrate binding and/or cleavage; and (vii) repeating steps (ii)-(vi) using the sequence identified in (vi).

Additional embodiments of the present methods, compounds, and compositions, and the like, will be apparent from the following description, drawings, examples, and claims. As can be appreciated from the foregoing and following description, each and every feature described herein, and each and every combination of two or more of such features, is included provided that the features included in such a combination are not mutually inconsistent. In addition, any feature or combination of features may be specifically excluded from any embodiment. Additional embodiments and advantages are set forth in the following description and claims, particularly when considered in conjunction with the accompanying examples and drawings.

### BRIEF DESCRIPTION OF THE DRAWINGS

FIG. 1A and FIG. 1B provide specificity data of exemplary botulinum neurotoxin light chain type A (BoNT LC/A) proteins (proteases). The Y-axis in FIG. 1A and FIG. 1B indicate the ratio of the rate of cleavage for SNAP23 to SNAP25 for each of the exemplary modified SNAP23-specific BoNT LC/A proteases identified herein as SEQ ID NOS. FIG. 1A demonstrates improved specificity of exemplary SNAP23-specific modified BoNT LC/A proteases for SNAP23 over SNAP25 in LC/A assay buffer (50 mM HEPES pH 7.4). FIG. 1B demonstrates improved specificity of two exemplary SNAP23-specific modified BoNT LC/A proteases for SNAP23 over SNAP25 in salt-containing intracellular buffer (50 mM KH₂PO₄ pH 7.4). Abbreviations used in the figures: qmLC/A is protease variant of SEQ ID NO: 27; SNAP23 substrate (SEQ ID NO: 24) is labeled as S23; SNAP25 substrate (SEQ ID NO: 25) is labeled as S25.

FIG. 2A and FIG. 2B demonstrate that an exemplary active wild-type (hereinafter “WT”) LC/A protease (SEQ ID NO: 1) was successfully displayed on the P8 protein of M13 bacteriophage (hereinafter “LC/A Φ” or “LC/A phage”) as determined by comparing the activity of a recombinantly expressed and purified WT LC/A (rLC/A) (shown in FIG. 2A) to the activity of LC/A Φ (shown in FIG. 2B). Vo refers to the initial rate of SNAP25 cleavage at various substrate concentrations, determined by monitoring changes in Fluorescence Units (FU) over time. Vₘₐₓ indicates the maximum rate of cleavage for each enzyme and Kₘ indicates the Michaelis-Menten constant.

FIG. 3A demonstrates SNAP23 and SNAP25 substrate binding by WT LC/A phage as determined by ELISA and FIG. 3B provides a schematic illustration of the complex formed by the binding of an anti-M13-HRP antibody, WT LC/A phage, and substrate. STOP4 phage with no displayed protein was used as a negative control for the assay.

FIG. 4A, FIG. 4B, and FIG. 4C provide specificity data of exemplary modified LC/A protease variants. The slopes of initial cleavage rates of SNAP25 (SEQ ID NO: 25) (FIG. 4A) and SNAP23 (SEQ ID NO: 24) (FIG. 4B) by the modified LC/A proteases were divided and normalized to the corresponding cleavage rates of the protease variant of SEQ ID NO: 27 (used as the reference protease) to determine improvements in SNAP23 specificity (FIG. 4C). The data demonstrates that the exemplary modified LC/A proteases represented in FIG. 4C, specifically the modified LC/A protease of SEQ ID NO: 11 (which includes a S254L substitution) was over 100-fold more specific and the modified LC/A protease of SEQ ID NO: 23 (which includes a S254M substitution) was over 40-fold more specific for SNAP23 over SNAP25 than the protease variant of SEQ ID NO: 27. The “mP” on y-axis represents fluorescence polarization in millipolarization units (mP) with time in seconds (s) on the x-axis.

FIG. 5A, FIG. 5B, FIG. 5C, and FIG. 5D provide specificity data of exemplary modified LC/A proteases generated with the error-prone PCR (epPCR) technique. Initial linear cleavage rates derived from the cleavage of SNAP25 (SEQ ID NO: 25) (shown in FIG. 5A) and SNAP23 (SEQ ID NO: 24) (shown in FIG. 5B) by the exemplary modified LC/A proteases were divided and normalized to the corresponding cleavage rates of the protease variant of SEQ ID NO: 27 to identify modified LC/A proteases with similar or improved SNAP23 specificity (shown in FIG. 5C). Selected modified LC/A proteases were screened again in triplicate to reveal that a modified LC/A protease with an N240S substitution (SEQ ID NO: 5) and a modified LC/A protease with the combination of an E201D and a D203V substitution (SEQ ID NO: 14) demonstrated improved SNAP23 specificity of 1.2- and 3.5-fold respectively over the protease variant of SEQ ID NO: 27 (shown in FIG. 5D). Abbreviations used in FIGS. 5A-5D: mP denotes Fluorescent Polarization; N240S denotes the modified LC/A protease of SEQ ID NO: 5; E201D/D203V denotes the modified LC/A protease of SEQ ID NO: 14; qmLC/A denotes the protease variant of SEQ ID NO: 27.

FIG. 6A, FIG. 6B and FIG. 6C provide specificity data of exemplary modified LC/A proteases generated with DNA shuffling technique. Initial linear cleavage rates derived from cleavage of SNAP25 (SEQ ID NO: 25) (shown in FIG. 6A) and SNAP23 (SEQ ID NO: 24) (shown in FIG. 6B) by the exemplary modified LC/A proteases were divided and normalized to the corresponding cleavage rates of the protease variant of SEQ ID NO: 27 (used as the reference protease) to determine which modified LC/A proteases have similar or improved SNAP23 specificity over the prior modified LC/A protease with N240S substitution (SEQ ID NO: 5) and LC/A protease with the combination of E201D and D203V substitutions (data shown in FIG. 6C). Abbreviations used in FIGS. 6A-6C: mP denotes Fluorescent Polarization; N240S denotes the modified LC/A protease of SEQ ID NO: 5; E201D/D203V denotes the modified LC/A protease of SEQ ID NO: 14; qmLC/A denotes the protease variant of SEQ ID NO: 27.

FIG. 7A and FIG. 7B demonstrate exemplary modified LC/E proteases (generated using LC/E protease of SEQ ID NO: 28) that cleave SNAP29 (SEQ ID NO: 4) and SNAP25/29 chimeric substrate (SEQ ID NO: 29). FIG. 7A demonstrates that the exemplary SNAP25/29 (SEQ ID NO: 29) chimeric substrate (19% SNAP25, 81% SNAP29) was cleaved by wild-type LC/E in a fluorescence-polarization assay. Trypsin was used as a positive control in the assay. FIG. 7B demonstrates that at least five exemplary modified LC/E proteases screened against both the SNAP25/29 chimeric substrate and SNAP25 showed improved specificity for the chimeric substrate. Abbreviations used in FIG. 7A and FIG. 7B: S29/25 chimera and S29/25 denote SNAP25/29 chimeric substrate (SEQ ID NO: 29); S25 denotes SNAP25 substrate (SEQ ID NO: 25); S29 denotes SNAP29 substrate (SEQ ID NO: 4).

FIG. 8A, FIG. 8B, FIG. 8C, FIG. 8D and FIG. 8E show exemplary data demonstrating a novel method for modulating substrate specificity of the BoNT proteases using zinc (Zn²⁺) as a co-factor. FIG. 8A: Purification of the modified LC/A protease (SEQ ID NO: 13) from cell lysate unexpectedly yielded a protease that cleaves SNAP25 approximately five (5) times faster than SNAP23 as assessed by a fluorescence-polarization assay. FIG. 8B: Purification of the modified LC/A protease (SEQ ID NO: 13) using a Zn-charged metal affinity chromatography resin instead of a nickel-charged resin in low-salt conditions (50 mM HEPES 100 mM NaCl pH 8.0, with 0, 20, or 250 mM imidazole) improved SNAP23 cleavage rate approximately 3.5-fold. FIG. 8C, FIG. 8D, and FIG. 8E: Dialysis of the modified LC/A (SEQ ID NO: 13) purified in low-salt conditions into zinc buffer (50 mM HEPES, 0.2 mM ZnCl₂, pH 7.4) (FIG. 8D) yielded a protease that cleaves SNAP23 approximately four (4) times faster than SNAP25. Individual rates for SNAP23 and SNAP25 for the purified LC/A protease (SEQ ID NO: 13) dialyzed into assay buffer (50 mM HEPES pH 7.4) or zinc buffer are shown in FIG. 8C and FIG. 8D, respectively. Dividing the initial rates yields the specificity index depicted in FIG. 8E. The data in these figures demonstrates that presence of Zn²⁺ recovered SNAP23 specificity of the modified proteases. For all data shown in FIGS. 8A-8E, the final concentration of the enzyme was 50 nM, and the final concentration of the substrate was 2 μM.

FIG. 9A, FIG. 9B, FIG. 9C, FIG. 9D, FIG. 9E, FIG. 9F, FIG. 9G, and FIG. 911 provide a compilation of amino acid sequences pertaining to various derivative proteases and molecules as described in the specification.

FIG. 10A, FIG. 10B, FIG. 10C, and FIG. 10D indicate that the modified BoNT/A disclosed herein specifically cleave SNAP23 while concomitantly having a reduced ability to cleave SNAP25. FIG. 10A shows the construction, expression, and purification of a modified full length BoNT/A (referred hereinafter as “omBoNT/A”) comprising the modified LC/A protease of SEQ ID NO: 13 (also referred to as “omLC/A”). Recombinant omBoNT/A was purified by immobilized metal affinity chromatography (IMAC) followed by anion exchange (AEX) chromatography. The omBoNT/A was about 95% nicked upon DTT reduction as demonstrated by the presence of the HC/A and omLC/A bands. FIG. 10B shows Western blots of in vitro cleaved human rSNAP23. Human rSNAP23 protein (30 μg) was incubated with 400 nM of either wild type LC/A, wild type LC/E, or reduced preparations of omBoNT/A toxin (1 and 2) at 37° C. for 1 hour in PBS, pH 7. In vitro cleavage of human rSNAP23 was visualized with a C-terminal anti-SNAP23 antibody (panel A) and a N-terminal anti-SNAP23 antibody (panel B). FIG. 10C shows Western blot analyses of SNAP23 and SNAP25 cleavage in SiMa cells treated with 50 nM of omBoNT/A or wtBoNT/A for 48 hours. SNAP23 cleavage (panel A) and SNAP25 cleavage (panel B) were visualized with antibodies to N-terminal SNAP23 or SNAP25, respectively. mCherry antibody was used as a loading control to ensure similar loading in all conditions. FIG. 10D summarizes the in vivo effect of omBoNT/A compared to wtBoNT/A on neuromuscular paralysis. FIG. 10D, panel A: Neuromuscular paralysis measured by peak DAS effect in mice (n=3/dose; N=3) injected with omBoNT/A (0.2, 1, or 5 ng/kg) or wtBoNT/A (0.2 ng/kg). The paralytic response is mediated by cleavage of SNAP25. FIG. 10D, panel B: Mice injected with omBoNT/A (n=3/dose; N=3) had no changes in the well-being score (WBS) over four days. These results confirmed reduced SNAP25 activity in vivo and demonstrated that omBoNT/A did not have increased toxicity compared to wtBoNT/A.

### BRIEF DESCRIPTION OF THE ABBREVIATIONS USED

| Abbreviation | Description |
|---|---|
| qmLC/A | A quadruple mutant of the light chain of botulinum neurotoxin |
| | type A having SEQ ID NO: 27 |
| BoNT | Botulinum neurotoxin |
| BoNT LC/A or LC/A | Botulinum neurotoxin type A light chain (LC) |
| BoNT LC/E or LC/E | Botulinum neurotoxin type E light chain (LC) |
| BoNT/A | Botulinum neurotoxin type A |
| BoNT/E | Botulinum neurotoxin type E |
| DARET assay | Depolarization after resonance energy transfer assay |
| epPCR | Error-prone PCR |
| H-chain or HC | C-terminal heavy chain (H-chain) of BoNTs. The H-chain |
| | consists of a translocation domain (H_{N}) and a C-terminal neuron- |
| | binding domain (H_{c}) |
| L-chain or LC | N-terminal proteolytic light chain of BoNTs |
| NSF | N-ethylmaleimide-Sensitive Factor |
| r | recombinantly expressed |
| SNAP | Synaptosomal-associated protein |
| SNARE protein | Soluble N-ethylmaleimide-sensitive factor (NSF) Attachment |
| | protein REceptor (e.g. SNAP-25, VAMP, or Syntaxin) protein |
| omLC/A | An octa mutant of the light chain of botulinum neurotoxin type |
| | A having SEQ ID NO: 13 |
| omBoNT/A | Modified full length BoNT/A comprising the modified LC/A |
| | protease variant of SEQ ID NO: 13 |
| WT | Wild type |
| | A wtBoNT/A or wt BoNT/E is a protein, native or recombinant, |
| | that has the same protein sequence as native Botulinum |
| | Neurotoxin Type A (BoNT/A) or native BoNT/E, respectively. |

### BRIEF DESCRIPTION OF THE SEQUENCES

-amino acid sequence of wild-type botulinum neurotoxin serotype A
(BONT/A) light chain (amino acid residues 1-448 of UniProt P0DPI1):
SEQ ID NO: 1
MPFVNKQFNYKDPVNGVDIAYIKIPNAGQMQPVKAFKIHNKIWWIPERDTFTNPEEGDLNPPPEAKQVPVSYYDST
YLSTDNEKDNYLKGVTKLFERIYSTDLGRMLLTSIVRGIPFWGGSTIDTELKVIDTNCINVIQPDGSYRSEELNLVIIG
PSADIIQFECKSFGHEVLNLTRNGYGSTQYIRFSPDFTFGFEESLEVDTNPLLGAGKFATDPAVTLAHELIHAGHRL
YGIAINPNRVFKVNTNAYYEMSGLEVSFEELRTFGGHDAKFIDSLQENEFRLYYYNKFKDIASTLNKAKSIVGTTAS
LQYMKNVFKEKYLLSEDTSGKFSVDKLKFDKLYKMLTEIYTEDNFVKFFKVLNRKTYLNFDKAVFKINIVPKVNYTIY
DGFNLRNTNLAANFNGQNTEINNMNFTKLKNFTGLFEFYKLLCVRGIITSKTKSLDKGYNK
-amino acid sequence of human SNAP23 of Uniprot O00161:
SEQ ID NO: 2
MDNLSSEEIQQRAHQITDESLESTRRILGLAIESQDAGIKTITMLDEQKEQLNRIEEGLDQINKDMRETEKTLTELNK
CCGLCVCPCNRTKNFESGKAYKTTWGDGGENSPCNVVSKQPGPVTNGQLQQPTTGAASGGYIKRITNDAREDE
MEENLTQVGSILGNLKDMALNIGNEIDAQNPQIKRITDKADTNRDRIDIANARAKKLIDS
-amino acid sequence of human and rodent SNAP25 of UniProt P60880:
SEQ ID NO: 3
MAEDADMRNELEEMQRRADQLADESLESTRRMLQLVEESKDAGIRTLVMLDEQGEQLERIEEGMDQINKDMKE
AEKNLTDLGKFCGLCVCPCNKLKSSDAYKKAWGNNQDGVVASQPARVVDEREQMAISGGFIRRVTNDARENEM
DENLEQVSGIIGNLRHMALDMGNEIDTQNRQIDRIMEKADSNKTRIDEANQRATKMLGSG
-amino acid sequence of human SNAP29:
SEQ ID NO: 4
MSAYPKSYNPFDDDGEDEGARPAPWRDARDLPDGPDAPADRQQYLRQEVLRRAEATAASTSRSLALMYESEK
VGVASSEELARQRGVLERTEKMVDKMDQDLKISQKHINSIKSVFGGLVNYFKSKPVETPPEQNGTLTSQPNNRLK
EAISTSKEQEAKYQASHPNLRKLDDTDPVPRGAGSAMSTDAYPKNPHLRAYHQKIDSNLDELSMGLGRLKDIALG
MQTEIEEQDDILDRLTTKVDKLDVNIKSTERKVRQL
-amino acid sequence of exemplary BoNT/A light chain variant with E148Y, K166F,
N240S, S254A, and G305D substitutions:
SEQ ID NO: 5
MPFVNKQFNYKDPVNGVDIAYIKIPNAGQMQPVKAFKIHNKIWWIPERDTFTNPEEGDLNPPPEAKQVPVSYYDST
YLSTDNEKDNYLKGVTKLFERIYSTDLGRMLLTSIVRGIPFWGGSTIDTELKVIDTNCINVIQPDGSYRSEYLNLVIIG
PSADIIQFECFSFGHEVLNLTRNGYGSTQYIRFSPDFTFGFEESLEVDTNPLLGAGKFATDPAVTLAHELIHAGHRL
YGIAINPSRVFKVNTNAYYEMAGLEVSFEELRTFGGHDAKFIDSLQENEFRLYYYNKFKDIASTLNKAKSIVDTTAS
LQYMKNVFKEKYLLSEDTSGKFSVDKLKFDKLYKMLTEIYTEDNFVKFFKVLNRKTYLNFDKAVFKINIVPKVNYTIY
DGFNLRNTNLAANFNGQNTEINNMNFTKLKNFTGLFEFYKLL
-amino acid sequence of exemplary BoNT/A light chain variant with
E148Y, K166F, N240A, S254A and G305D substitutions:
SEQ ID NO: 6
MPFVNKQFNYKDPVNGVDIAYIKIPNAGQMQPVKAFKIHNKIWWIPERDTFTNPEEGDLNPPPEAKQVPVSYYDST
YLSTDNEKDNYLKGVTKLFERIYSTDLGRMLLTSIVRGIPFWGGSTIDTELKVIDTNCINVIQPDGSYRSEYLNLVIIG
PSADIIQFECFSFGHEVLNLTRNGYGSTQYIRFSPDFTFGFEESLEVDTNPLLGAGKFATDPAVTLAHELIHAGHRL
YGIAINPARVFKVNTNAYYEMAGLEVSFEELRTFGGHDAKFIDSLQENEFRLYYYNKFKDIASTLNKAKSIVDTTAS
LQYMKNVFKEKYLLSEDTSGKFSVDKLKFDKLYKMLTEIYTEDNFVKFFKVLNRKTYLNFDKAVFKINIVPKVNYTIY
DGFNLRNTNLAANFNGQNTEINNMNFTKLKNFTGLFEFYKLL
-amino acid sequence of exemplary BoNT/A light chain variant with N26S, E148Y,
K166F, N240A, S254A and G305D substitutions:
SEQ ID NO: 7
MPFVNKQFNYKDPVNGVDIAYIKIPSAGQMQPVKAFKIHNKIWWIPERDTFTNPEEGDLNPPPEAKQVPVSYYDST
YLSTDNEKDNYLKGVTKLFERIYSTDLGRMLLTSIVRGIPFWGGSTIDTELKVIDTNCINVIQPDGSYRSEYLNLVIIG
PSADIIQFECFSFGHEVLNLTRNGYGSTQYIRFSPDFTFGFEESLEVDTNPLLGAGKFATDPAVTLAHELIHAGHRL
YGIAINPARVFKVNTNAYYEMAGLEVSFEELRTFGGHDAKFIDSLQENEFRLYYYNKFKDIASTLNKAKSIVDTTAS
LQYMKNVFKEKYLLSEDTSGKFSVDKLKFDKLYKMLTEIYTEDNFVKFFKVLNRKTYLNFDKAVFKINIVPKVNYTIY
DGFNLRNTNLAANFNGQNTEINNMNFTKLKNFTGLFEFYKLL
-amino acid sequence of exemplary BoNT/A light chain variant with
N26S, E55V, E148Y, K166F, N240A, S254A and G305D substitutions:
SEQ ID NO: 8
MPFVNKQFNYKDPVNGVDIAYIKIPSAGQMQPVKAFKIHNKIWWIPERDTFTNPVEGDLNPPPEAKQVPVSYYDST
YLSTDNEKDNYLKGVTKLFERIYSTDLGRMLLTSIVRGIPFWGGSTIDTELKVIDTNCINVIQPDGSYRSEYLNLVIIG
PSADIIQFECFSFGHEVLNLTRNGYGSTQYIRFSPDFTFGFEESLEVDTNPLLGAGKFATDPAVTLAHELIHAGHRL
YGIAINPARVFKVNTNAYYEMAGLEVSFEELRTFGGHDAKFIDSLQENEFRLYYYNKFKDIASTLNKAKSIVDTTAS
LQYMKNVFKEKYLLSEDTSGKFSVDKLKFDKLYKMLTEIYTEDNFVKFFKVLNRKTYLNFDKAVFKINIVPKVNYTIY
DGFNLRNTNLAANFNGQNTEINNMNFTKLKNFTGLFEFYKLL
-amino acid sequence of exemplary BoNT/A light chain variant with
N26S, Q29R, E55V, E148Y, K166F, N240A, S254A and G305D substitutions:
SEQ ID NO: 9
MPFVNKQFNYKDPVNGVDIAYIKIPSAGRMQPVKAFKIHNKIWWIPERDTFTNPVEGDLNPPPEAKQVPVSYYDST
YLSTDNEKDNYLKGVTKLFERIYSTDLGRMLLTSIVRGIPFWGGSTIDTELKVIDTNCINVIQPDGSYRSEYLNLVIIG
PSADIIQFECFSFGHEVLNLTRNGYGSTQYIRFSPDFTFGFEESLEVDTNPLLGAGKFATDPAVTLAHELIHAGHRL
YGIAINPARVFKVNTNAYYEMAGLEVSFEELRTFGGHDAKFIDSLQENEFRLYYYNKFKDIASTLNKAKSIVDTTAS
LQYMKNVFKEKYLLSEDTSGKFSVDKLKFDKLYKMLTEIYTEDNFVKFFKVLNRKTYLNFDKAVFKINIVPKVNYTIY
DGFNLRNTNLAANFNGQNTEINNMNFTKLKNFTGLFEFYKLL
-amino acid sequence of exemplary BoNT/A light chain variant
with N26S, Q29R, N53R, E55V, E148Y, K166F, N240A, S254A and G305D substitutions:
SEQ ID NO: 10
MPFVNKQFNYKDPVNGVDIAYIKIPSAGRMQPVKAFKIHNKIWIPERDTFTRPVEGDLNPPPEAKQVPVSYYDST
YLSTDNEKDNYLKGVTKLFERIYSTDLGRMLLTSIVRGIPFWGGSTIDTELKVIDTNCINVIQPDGSYRSEYLNLVIIG
PSADIIQFECFSFGHEVLNLTRNGYGSTQYIRFSPDFTFGFEESLEVDTNPLLGAGKFATDPAVTLAHELIHAGHRL
YGIAINPARVFKVNTNAYYEMAGLEVSFEELRTFGGHDAKFIDSLQENEFRLYYYNKFKDIASTLNKAKSIVDTTAS
LQYMKNVFKEKYLLSEDTSGKFSVDKLKFDKLYKMLTEIYTEDNFVKFFKVLNRKTYLNFDKAVFKINIVPKVNYTIY
DGFNLRNTNLAANFNGQNTEINNMNFTKLKNFTGLFEFYKLL
-amino acid sequence of exemplary BoNT/A light chain variant
with N26S, Q29R, N53R, E55V, E148Y, K166F, N240A, S254L and G305D substitutions:
SEQ ID NO: 11
MPFVNKQFNYKDPVNGVDIAYIKIPSAGRMQPVKAFKIHNKIWWIPERDTFTRPVEGDLNPPPEAKQVPVSYYDST
YLSTDNEKDNYLKGVTKLFERIYSTDLGRMLLTSIVRGIPFWGGSTIDTELKVIDTNCINVIQPDGSYRSEYLNLVIIG
PSADIIQFECFSFGHEVLNLTRNGYGSTQYIRFSPDFTFGFEESLEVDTNPLLGAGKFATDPAVTLAHELIHAGHRL
YGIAINPARVFKVNTNAYYEMLGLEVSFEELRTFGGHDAKFIDSLQENEFRLYYYNKFKDIASTLNKAKSIVDTTAS
LQYMKNVFKEKYLLSEDTSGKFSVDKLKFDKLYKMLTEIYTEDNFVKFFKVLNRKTYLNFDKAVFKINIVPKVNYTIY
DGFNLRNTNLAANFNGQNTEINNMNFTKLKNFTGLFEFYKLL
-amino acid sequence of exemplary BoNT/A light chain variant
with N26S, Q29R, N53R, E55V, E148Y, K166F, N240A and S254L substitutions:
SEQ ID NO: 12
MPFVNKQFNYKDPVNGVDIAYIKIPSAGRMQPVKAFKIHNKIWWIPERDTFTRPVEGDLNPPPEAKQVPVSYYDST
YLSTDNEKDNYLKGVTKLFERIYSTDLGRMLLTSIVRGIPFWGGSTIDTELKVIDTNCINVIQPDGSYRSEYLNLVIIG
PSADIIQFECFSFGHEVLNLTRNGYGSTQYIRFSPDFTFGFEESLEVDTNPLLGAGKFATDPAVTLAHELIHAGHRL
YGIAINPARVFKVNTNAYYEMLGLEVSFEELRTFGGHDAKFIDSLQENEFRLYYYNKFKDIASTLNKAKSIVGTTAS
LQYMKNVFKEKYLLSEDTSGKFSVDKLKFDKLYKMLTEIYTEDNFVKFFKVLNRKTYLNFDKAVFKINIVPKVNYTIY
DGFNLRNTNLAANFNGQNTEINNMNFTKLKNFTGLFEFYKLL
-amino acid sequence of exemplary BoNT/A light chain variant
with N26S, Q29R, N53H, E55V, E148Y, K166, N240A and S254L substitutions:
SEQ ID NO: 13
MPFVNKQFNYKDPVNGVDIAYIKIPSAGRMQPVKAFKIHNKIWWIPERDTFTHPVEGDLNPPPEAKQVPVSYYDST
YLSTDNEKDNYLKGVTKLFERIYSTDLGRMLLTSIVRGIPFWGGSTIDTELKVIDTNCINVIQPDGSYRSEYLNLVIIG
PSADIIQFECFSFGHEVLNLTRNGYGSTQYIRFSPDFTFGFEESLEVDTNPLLGAGKFATDPAVTLAHELIHAGHRL
YGIAINPARVFKVNTNAYYEMLGLEVSFEELRTFGGHDAKFIDSLQENEFRLYYYNKFKDIASTLNKAKSIVGTTAS
LQYMKNVFKEKYLLSEDTSGKFSVDKLKFDKLYKMLTEIYTEDNFVKFFKVLNRKTYLNFDKAVFKINIVPKVNYTIY
DGFNLRNTNLAANFNGQNTEINNMNFTKLKNFTGLFEFYKLL
-amino acid sequence of exemplary BoNT/A light chain variant
with E148Y, K166F, E201D, D203V, S254A and G305D substitutions:
SEQ ID NO: 14
MPFVNKQFNYKDPVNGVDIAYIKIPNAGQMQPVKAFKIHNKIWWIPERDTFTNPEEGDLNPPPEAKQVPVSYYDST
YLSTDNEKDNYLKGVTKLFERIYSTDLGRMLLTSIVRGIPFWGGSTIDTELKVIDTNCINVIQPDGSYRSEYLNLVIIG
PSADIIQFECFSFGHEVLNLTRNGYGSTQYIRFSPDFTFGFEESLDVVTNPLLGAGKFATDPAVTLAHELIHAGHRL
YGIAINPNRVFKVNTNAYYEMAGLEVSFEELRTFGGHDAKFIDSLQENEFRLYYYNKFKDIASTLNKAKSIVDTTAS
LQYMKNVFKEKYLLSEDTSGKFSVDKLKFDKLYKMLTEIYTEDNFVKFFKVLNRKTYLNFDKAVFKINIVPKVNYTIY
DGFNLRNTNLAANFNGQNTEINNMNFTKLKNFTGLFEFYKLL
-amino acid sequence of exemplary BoNT/A light chain variant
with E148Y, K166F, S254A, G305D, K364R and Y387N substitutions:
SEQ ID NO: 15
MPFVNKQFNYKDPVNGVDIAYIKIPNAGQMQPVKAFKIHNKIWWIPERDTFTNPEEGDLNPPPEAKQVPVSYYDST
YLSTDNEKDNYLKGVTKLFERIYSTDLGRMLLTSIVRGIPFWGGSTIDTELKVIDTNCINVIQPDGSYRSEYLNLVIIG
PSADIIQFECFSFGHEVLNLTRNGYGSTQYIRFSPDFTFGFEESLEVDTNPLLGAGKFATDPAVTLAHELIHAGHRL
YGIAINPNRVFKVNTNAYYEMAGLEVSFEELRTFGGHDAKFIDSLQENEFRLYYYNKFKDIASTLNKAKSIVDTTAS
LQYMKNVFKEKYLLSEDTSGKFSVDKLKFDKLYKMLTEIYTEDNFVKFFKVLNRRTYLNFDKAVFKINIVPKVNYTI
NDGFNLRNTNLAANFNGQNTEINNMNFTKLKNFTGLFEFYKLL
-amino acid sequence of exemplary BoNT/A light chain variant
with N26S, E148Y, Q162R, K166F, N240A, S254A and G305D substitutions:
SEQ ID NO: 16
MPFVNKQFNYKDPVNGVDIAYIKIPSAGQMQPVKAFKIHNKIWIPERDTFTNPEEGDLNPPPEAKQVPVSYYDST
YLSTDNEKDNYLKGVTKLFERIYSTDLGRMLLTSIVRGIPFWGGSTIDTELKVIDTNCINVIQPDGSYRSEYLNLVIIG
PSADIIRFECFSFGHEVLNLTRNGYGSTQYIRFSPDFTFGFEESLEVDTNPLLGAGKFATDPAVTLAHELIHAGHRL
YGIAINPARVFKVNTNAYYEMAGLEVSFEELRTFGGHDAKFIDSLQENEFRLYYYNKFKDIASTLNKAKSIVDTTAS
LQYMKNVFKEKYLLSEDTSGKFSVDKLKFDKLYKMLTEIYTEDNFVKFFKVLNRKTYLNFDKAVFKINIVPKVNYTIY
DGFNLRNTNLAANFNGQNTEINNMNFTKLKNFTGLFEFYKLL
-amino acid sequence of exemplary BoNT/A light chain variant
with N26S, E55N, E148Y, K166F, N240A, S254A and G305D substitutions:
SEQ ID NO: 17
MPFVNKQFNYKDPVNGVDIAYIKIPSAGQMQPVKAFKIHNKIWWIPERDTFTNPNEGDLNPPPEAKQVPVSYYDST
YLSTDNEKDNYLKGVTKLFERIYSTDLGRMLLTSIVRGIPFWGGSTIDTELKVIDTNCINVIQPDGSYRSEYLNLVIIG
PSADIIQFECFSFGHEVLNLTRNGYGSTQYIRFSPDFTFGFEESLEVDTNPLLGAGKFATDPAVTLAHELIHAGHRL
YGIAINPARVFKVNTNAYYEMAGLEVSFEELRTFGGHDAKFIDSLQENEFRLYYYNKFKDIASTLNKAKSIVDTTAS
LQYMKNVFKEKYLLSEDTSGKFSVDKLKFDKLYKMLTEIYTEDNFVKFFKVLNRKTYLNFDKAVFKINIVPKVNYTIY
DGFNLRNTNLAANFNGQNTEINNMNFTKLKNFTGLFEFYKLL
-amino acid sequence of exemplary BoNT/A light chain variant
with N26S, E55I, E148Y, K166F, N240A, S254A and G305D substitutions:
SEQ ID NO: 18
MPFVNKQFNYKDPVNGVDIAYIKIPSAGQMQPVKAFKIHNKIWWIPERDTFTNPIEGDLNPPPEAKQVPVSYYDST
YLSTDNEKDNYLKGVTKLFERIYSTDLGRMLLTSIVRGIPFWGGSTIDTELKVIDTNCINVIQPDGSYRSEYLNLVIIG
PSADIIQFECFSFGHEVLNLTRNGYGSTQYIRFSPDFTFGFEESLEVDTNPLLGAGKFATDPAVTLAHELIHAGHRL
YGIAINPARVFKVNTNAYYEMAGLEVSFEELRTFGGHDAKFIDSLQENEFRLYYYNKFKDIASTLNKAKSIVDTTAS
LQYMKNVFKEKYLLSEDTSGKFSVDKLKFDKLYKMLTEIYTEDNFVKFFKVLNRKTYLNFDKAVFKINIVPKVNYTIY
DGFNLRNTNLAANFNGQNTEINNMNFTKLKNFTGLFEFYKLL
-amino acid sequence of exemplary BoNT/A light chain variant
with N26S, Q29S, E55V, E148Y, K166F, N240A, S254A and G305D substitutions:
SEQ ID NO: 19
MPFVNKQFNYKDPVNGVDIAYIKIPSAGSMQPVKAFKIHNKIWWIPERDTFTNPVEGDLNPPPEAKQVPVSYYDST
YLSTDNEKDNYLKGVTKLFERIYSTDLGRMLLTSIVRGIPFWGGSTIDTELKVIDTNCINVIQPDGSYRSEYLNLVIIG
PSADIIQFECFSFGHEVLNLTRNGYGSTQYIRFSPDFTFGFEESLEVDTNPLLGAGKFATDPAVTLAHELIHAGHRL
YGIAINPARVFKVNTNAYYEMAGLEVSFEELRTFGGHDAKFIDSLQENEFRLYYYNKFKDIASTLNKAKSIVDTTAS
LQYMKNVFKEKYLLSEDTSGKFSVDKLKFDKLYKMLTEIYTEDNFVKFFKVLNRKTYLNFDKAVFKINIVPKVNYTIY
DGFNLRNTNLAANFNGQNTEINNMNFTKLKNFTGLFEFYKLL
-amino acid sequence of exemplary BoNT/A light chain variant with N26S, A27L,
Q29R, N53R, E55V, E148Y, K166F, N240A, S254A and G305D substitutions:
SEQ ID NO: 20
MPFVNKQFNYKDPVNGVDIAYIKIPSLGRMQPVKAFKIHNKIWIPERDTFTRPVEGDLNPPPEAKQVPVSYYDST
YLSTDNEKDNYLKGVTKLFERIYSTDLGRMLLTSIVRGIPFWGGSTIDTELKVIDTNCINVIQPDGSYRSEYLNLVIIG
PSADIIQFECFSFGHEVLNLTRNGYGSTQYIRFSPDFTFGFEESLEVDTNPLLGAGKFATDPAVTLAHELIHAGHRL
YGIAINPARVFKVNTNAYYEMAGLEVSFEELRTFGGHDAKFIDSLQENEFRLYYYNKFKDIASTLNKAKSIVDTTAS
LQYMKNVFKEKYLLSEDTSGKFSVDKLKFDKLYKMLTEIYTEDNFVKFFKVLNRKTYLNFDKAVFKINIVPKVNYTIY
DGFNLRNTNLAANFNGQNTEINNMNFTKLKNFTGLFEFYKLL
-amino acid sequence of exemplary BoNT/A light chain variant with N26S, A27R,
Q29R, N53R, E55V, E148Y, K166F, N240A, S254A and G305D substitutions:
SEQ ID NO: 21
MPFVNKQFNYKDPVNGVDIAYIKIPSRGRMQPVKAFKIHNKIWIPERDTFTRPVEGDLNPPPEAKQVPVSYYDST
YLSTDNEKDNYLKGVTKLFERIYSTDLGRMLLTSIVRGIPFWGGSTIDTELKVIDTNCINVIQPDGSYRSEYLNLVIIG
PSADIIQFECFSFGHEVLNLTRNGYGSTQYIRFSPDFTFGFEESLEVDTNPLLGAGKFATDPAVTLAHELIHAGHRL
YGIAINPARVFKVNTNAYYEMAGLEVSFEELRTFGGHDAKFIDSLQENEFRLYYYNKFKDIASTLNKAKSIVDTTAS
LQYMKNVFKEKYLLSEDTSGKFSVDKLKFDKLYKMLTEIYTEDNFVKFFKVLNRKTYLNFDKAVFKINIVPKVNYTIY
DGFNLRNTNLAANFNGQNTEINNMNFTKLKNFTGLFEFYKLL
-amino acid sequence of exemplary BoNT/A light chain variant with N26S, Q29R,
N53R, E55V, E56I, E148Y, K166F, N240A,S254A and G305D substitutions:
SEQ ID NO: 22
MPFVNKQFNYKDPVNGVDIAYIKIPSAGRMQPVKAFKIHNKIWWIPERDTFTRPVIGDLNPPPEAKQVPVSYYDST
YLSTDNEKDNYLKGVTKLFERIYSTDLGRMLLTSIVRGIPFWGGSTIDTELKVIDTNCINVIQPDGSYRSEYLNLVIIG
PSADIIQFECFSFGHEVLNLTRNGYGSTQYIRFSPDFTFGFEESLEVDTNPLLGAGKFATDPAVTLAHELIHAGHRL
YGIAINPARVFKVNTNAYYEMAGLEVSFEELRTFGGHDAKFIDSLQENEFRLYYYNKFKDIASTLNKAKSIVDTTAS
LQYMKNVFKEKYLLSEDTSGKFSVDKLKFDKLYKMLTEIYTEDNFVKFFKVLNRKTYLNFDKAVFKINIVPKVNYTIY
DGFNLRNTNLAANFNGQNTEINNMNFTKLKNFTGLFEFYKLL
-amino acid sequence of exemplary BoNT/A light chain variant
with N26S, Q29R, N53R, E55V, E148Y, K166F, N240A, S254M and G305D substitutions:
SEQ ID NO: 23
MPFVNKQFNYKDPVNGVDIAYIKIPSAGRMQPVKAFKIHNKIWWIPERDTFTRPVEGDLNPPPEAKQVPVSYYDST
YLSTDNEKDNYLKGVTKLFERIYSTDLGRMLLTSIVRGIPFWGGSTIDTELKVIDTNCINVIQPDGSYRSEYLNLVIIG
PSADIIQFECFSFGHEVLNLTRNGYGSTQYIRFSPDFTFGFEESLEVDTNPLLGAGKFATDPAVTLAHELIHAGHRL
YGIAINPARVFKVNTNAYYEMMGLEVSFEELRTFGGHDAKFIDSLQENEFRLYYYNKFKDIASTLNKAKSIVDTTAS
LQYMKNVFKEKYLLSEDTSGKFSVDKLKFDKLYKMLTEIYTEDNFVKFFKVLNRKTYLNFDKAVFKINIVPKVNYTIY
DGFNLRNTNLAANFNGQNTEINNMNFTKLKNFTGLFEFYKLL
-amino acid sequence of SNAP23 substrate:
SEQ ID NO: 24
MHHHHHHENLYFQGIFRAPMASKGEELFTGVVPILVELDGDVNGHKFSVSGEGEGDATYGKLTLKFICTTGKLPV
PWPTLVTTLCYGVQCFSRYPDHMKRHDFFKSAMPEGYVQERTIFFKDDGNYKTRAEVKFEGDTLVNRIELKGIDF
KEDGNILGHKLEYNYNSHNVYIMADKQKNGIKVNFKTRHNIEDGSVQLADHYQQNTPIGDGPVLLPDNHYLSTQS
ALSKDPNEKRDHMVLLEFVTAAGITHGMDELYNGGAGSGAGGGGYIKRITNDAREDEMEENLTQVGSILGNLKD
MALNIGNEIDAQNPQIKRITDKADTNRDRIDIANARAKKLIDSGGGSSASKGEELFTGVVPILVELDGDVNGHKFSV
SGEGGGDATYGKLTLKFICTTGKLPVPWPTLVTTLSHGVQCFSRYPDHMKRHDFFKSAMPEGYVQERTIFFKDD
GNYKTRAEVKFEGDTLVNRIELKGIDFKEDGNILGHKLEYNYNSHNVYIMADKQKNGIKANFKIRHNIEDGSVQLAD
HYQQNTPIGDGPVLLPDNHYLSTQSALSKDPNEKRDHMVLLEFVTAAGITHGMDELYK
-amino acid sequence of SNAP25 substrate:
SEQ ID NO: 25
MHHHHHHENLYFQGIFRAPMASKGEELFTGVVPILVELDGDVNGHKFSVSGEGEGDATYGKLTLKFICTTGKLPV
PWPTLVTTLCYGVQCFSRYPDHMKRHDFFKSAMPEGYVQERTIFFKDDGNYKTRAEVKFEGDTLVNRIELKGIDF
KEDGNILGHKLEYNYNSHNVYIMADKQKNGIKVNFKTRHNIEDGSVQLADHYQQNTPIGDGPVLLPDNHYLSTQS
ALSKDPNEKRDHMVLLEFVTAAGITHGMDELYNGGAGSGAGGGGIRRVTNDARENEMDENLEQVSGIIGNLRHM
ALDMGNEIDTQNRQIDRIMEKADSNKTRIDEANQRATKMLGSGGGGGTASKGEELFTGVVPILVELDGDVNGHKF
SVSGEGEGDATYGKLTLKFICTTGKLPVPWPTLVTTLSHGVQCFSRYPDHMKRHDFFKSAMPEGYVQERTIFFK
DDGNYKTRAEVKFEGDTLVNRIELKGIDFKEDGNILGHKLEYNYNSHNVYIMADKQKNGIKANFKIRHNIEDGSVQ
LADHYQQNTPIGDGPVLLPDNHYLSTQSALSKDPNEKRDHMVLLEFVTAAGITHGMDELYK
-amino acid sequence of SNAP29 substrate:
SEQ ID NO: 26
MHHHHHHENLYFQGIFRAPMASKGEELFTGVVPILVELDGDVNGHKFSVSGEGEGDATYGKLTLKFICTTGKLPV
PWPTLVTTLCYGVQCFSRYPDHMKRHDFFKSAMPEGYVQERTIFFKDDGNYKTRAEVKFEGDTLVNRIELKGIDF
KEDGNILGHKLEYNYNSHNVYIMADKQKNGIKVNFKTRHNIEDGSVQLADHYQQNTPIGDGPVLLPDNHYLSTQS
ALSKDPNEKRDHMVLLEFVTAAGITHGMDELYNGGAGSGAGGSTDAYPKNPHLRAYHQKIDSNLDELSMGLGRL
KDIALGMQTEIEEQDDILDRLTTKVDKLDVNIKSTERKVRQLGGGSSVSKGEELFTGVVPILVELDGDVNGHKFSV
SGEGGGDATYGKLTLKFICTTGKLPVPWPTLVTTLSHGVQCFSRYPDHMKRHDFFKSAMPEGYVQERTIFFKDD
GNYKTRAEVKFEGDTLVNRIELKGIDFKEDGNILGHKLEYNYNSHNVYIMADKQKNGIKANFKIRHNIEDGSVQLAD
HYQQNTPIGDGPVLLPDNHYLSTQSALSKDPNEKRDHMVLLEFVTAAGITHGMDELYK
-amino acid sequence of BoNT/A light chain quadruple mutant
(“qmLC/A”) with E148Y, K166F, S254A, and G305D substitutions:
SEQ ID NO: 27
MPFVNKQFNYKDPVNGVDIAYIKIPNAGQMQPVKAFKIHNKIWWIPERDTFTNPEEGDLNPPPEAKQVPVSYYDST
YLSTDNEKDNYLKGVTKLFERIYSTDLGRMLLTSIVRGIPFWGGSTIDTELKVIDTNCINVIQPDGSYRSEYLNLVIIG
PSADIIQFECFSFGHEVLNLTRNGYGSTQYIRFSPDFTFGFEESLEVDTNPLLGAGKFATDPAVTLAHELIHAGHRL
YGIAINPNRVFKVNTNAYYEMAGLEVSFEELRTFGGHDAKFIDSLQENEFRLYYYNKFKDIASTLNKAKSIVDTTAS
LQYMKNVFKEKYLLSEDTSGKFSVDKLKFDKLYKMLTEIYTEDNFVKFFKVLNRKTYLNFDKAVFKINIVPKVNYTIY
DGFNLRNTNLAANFNGQNTEINNMNFTKLKNFTGLFEFYKLL
-amino acid sequence of BoNT/E light chain with three (3) amino
acid differences from the amino acid residues 1-411 of UniProt Q00496:
SEQ ID NO: 28
MPKINSFNYNDPVNDRTILYIKPGGCQEFYKSFNIMKNIWIIPERNVIGTTPQDFHPPTSLKNGDSSYYDPNYLQSD
EEKDRFLKIVTKIFNRINNNLSGGILLEELSKANPYLGNDNTPDNQFHIGDASAVEIKFSNGSQDILLPNVIIMGAEPD
LFETNSSNISLRNNYMPSNHGFGSIAIVTFSPEYSFRFNDNSMNEFIQDPALTLMHELIHSLHGLYGAKGITTKYTIT
QKQNPLITNIRGTNIEEFLTFGGTDLNIITSAQSNDIYTNLLADYKKIASKLSKVQVSNPLLNPYKDVFEAKYGLDKD
ASGIYSVNINKFNDIFKKLYSFTEFDLATKFQVKCRQTYIGQYKYFKLSNLLNDSIYNISEGYNINNLKVNFRGQNAN
LNPRIITPITGRGLVKKIIRF
-amino acid sequence of SNAP25/29 chimeric substrate:
SEQ ID NO: 29
MHHHHHHENLYFQGIFRAPMASKGEELFTGVVPILVELDGDVNGHKFSVSGEGEGDATYGKLTLKFICTTGKLPV
PWPTLVTTLCYGVQCFSRYPDHMKRHDFFKSAMPEGYVQERTIFFKDDGNYKTRAEVKFEGDTLVNRIELKGIDF
KEDGNILGHKLEYNYNSHNVYIMADKQKNGIKVNFKTRHNIEDGSVQLADHYQQNTPIGDGPVLLPDNHYLSTQS
ALSKDPNEKRDHMVLLEFVTAAGITHGMDELYNGGAGSGAGGSTDAYPKNPHLRAYHQKIDSNLDELSMGLGRL
KDIALGMGNEIDTQNRQIDRIMEKADKLDVNIKSTERKVRQLGGGSSVSKGEELFTGVVPILVELDGDVNGHKFSV
SGEGGGDATYGKLTLKFICTTGKLPVPWPTLVTTLSHGVQCFSRYPDHMKRHDFFKSAMPEGYVQERTIFFKDD
GNYKTRAEVKFEGDTLVNRIELKGIDFKEDGNILGHKLEYNYNSHNVYIMADKQKNGIKANFKIRHNIEDGSVQLAD
HYQQNTPIGDGPVLLPDNHYLSTQSALSKDPNEKRDHMVLLEFVTAAGITHGMDELYK
-amino acid sequence of full-length wild-type BoNT/A (amino acid residues 1-1296
of UniProt P0DPI1):
SEQ ID NO: 30
MPFVNKQFNYKDPVNGVDIAYIKIPNAGQMQPVKAFKIHNKIWWIPERDTFTNPEEGDLNPPPEAKQVPVSYYDST
YLSTDNEKDNYLKGVTKLFERIYSTDLGRMLLTSIVRGIPFWGGSTIDTELKVIDTNCINVIQPDGSYRSEELNLVIIG
PSADIIQFECKSFGHEVLNLTRNGYGSTQYIRFSPDFTFGFEESLEVDTNPLLGAGKFATDPAVTLAHELIHAGHRL
YGIAINPNRVFKVNTNAYYEMSGLEVSFEELRTFGGHDAKFIDSLQENEFRLYYYNKFKDIASTLNKAKSIVGTTAS
LQYMKNVFKEKYLLSEDTSGKFSVDKLKFDKLYKMLTEIYTEDNFVKFFKVLNRKTYLNFDKAVFKINIVPKVNYTIY
DGFNLRNTNLAANFNGQNTEINNMNFTKLKNFTGLFEFYKLLCVRGIITSKTKSLDKGYNKALNDLCIKVNNWDLF
FSPSEDNFTNDLNKGEEITSDTNIEAAEENISLDLIQQYYLTFNFDNEPENISIENLSSDIIGQLELMPNIERFPNGKK
YELDKYTMFHYLRAQEFEHGKSRIALTNSVNEALLNPSRVYTFFSSDYVKKVNKATEAAMFLGWWEQLVYDFTDE
TSEVSTTDKIADITIIIPYIGPALNIGNMLYKDDFVGALIFSGAVILLEFIPEIAIPVLGTFALVSYIANKVLTVQTIDNALS
KRNEKWDEVYKYIVTNWLAKVNTQIDLIRKKMKEALENQAEATKAIINYQYNQYTEEEKNNINFNIDDLSSKLNESI
NKAMININKFLNQCSVSYLMNSMIPYGVKRLEDFDASLKDALLKYIYDNRGTLIGQVDRLKDKVNNTLSTDIPFQLS
KYVDNQRLLSTFTEYIKNIINTSILNLRYESNHLIDLSRYASKINIGSKVNFDPIDKNQIQLFNLESSKIEVILKNAIVYN
SMYENFSTSFWIRIPKYFNSISLNNEYTIINCMENNSGWKVSLNYGEIIWTLQDTQEIKQRVVFKYSQMINISDYINR
WIFVTITNNRLNNSKIYINGRLIDQKPISNLGNIHASNNIMFKLDGCRDTHRYIWIKYFNLFDKELNEKEIKDLYDNQS
NSGILKDFWGDYLQYDKPYYMLNLYDPNKYVDVNNVGIRGYMYLKGPRGSVMTTNIYLNSSLYRGTKFIIKKYAS
GNKDNIVRNNDRVYINVVVKNKEYRLATNASQAGVEKILSALEIPDVGNLSQVVVMKSKNDQGITNKCKMNLQDN
NGNDIGFIGFHQFNNIAKLVASNWYNRQIERSSRTLGCSWEFIPVDDGWGERPL
-amino acid residues 1-411 of wild-type BoNT/E light chain from UniProt Q00496:
SEQ ID NO: 31
MPKINSFNYNDPVNDRTILYIKPGGCQEFYKSFNIMKNIWIIPERNVIGTTPQDFHPPTSLKNGDSSYYDPNYLQSD
EEKDRFLKIVTKIFNRINNNLSGGILLEELSKANPYLGNDNTPDNQFHIGDASAVEIKFSNGSQDILLPNVIIMGAEPD
LFETNSSNISLRNNYMPSNHRFGSIAIVTFSPEYSFRFNDNCMNEFIQDPALTLMHELIHSLHGLYGAKGITTKYTIT
QKQNPLITNIRGTNIEEFLTFGGTDLNIITSAQSNDIYTNLLADYKKIASKLSKVQVSNPLLNPYKDVFEAKYGLDKD
ASGIYSVNINKFNDIFKKLYSFTEFDLRTKFQVKCRQTYIGQYKYFKLSNLLNDSIYNISEGYNINNLKVNFRGQNAN
LNPRIITPITGRGLVKKIIRF
-amino acid sequence of activation loop of BoNT/A protease:
SEQ ID NO: 32
CVRGIITSKTKSLDKGYNKALNDLC

### DETAILED DESCRIPTION

### I. Definitions

Where a range of values is provided, it is intended that each intervening value between the upper and lower limit of that range and any other stated or intervening value in that stated range is encompassed, unless the context clearly dictates otherwise. Each smaller range between any stated value or intervening value in a stated range and any other stated or intervening value in that stated range is encompassed. The upper and lower limits of these smaller ranges may independently be included or excluded in the range, and each range where either, neither or both limits are included in the smaller ranges is also encompassed, subject to any specifically excluded limit in the stated range. Where the stated range includes one or both of the limits, ranges excluding either or both of those included limits are also within the scope. For example, if a range of 1 μm to 8 μm is stated, it is intended that 2 μm, 3 μm, 4 μm, 5 μm, 6 μm, and 7 μm are also explicitly disclosed, as well as the range of values greater than or equal to 1 μm and the range of values less than or equal to 8 μm.

The singular forms “a,” “an,” and “the” include plural referents unless the context clearly dictates otherwise. Thus, for example, reference to a “polymer” includes a single polymer as well as two or more of the same or different polymers, reference to an “excipient” includes a single excipient as well as two or more of the same or different excipients, and the like.

The term “about”, particularly in reference to a given quantity, is meant to encompass deviations of plus or minus five percent.

The term “administration”, or “to administer” means the step of giving (e.g., administering) a pharmaceutical composition to a subject, or alternatively a subject receiving a pharmaceutical composition. The pharmaceutical compositions disclosed herein can be locally administered by various methods. For example, intramuscular, intradermal, subcutaneous administration, intrathecal administration, intraperitoneal administration, topical (transdermal), instillation, and implantation (for example, of a slow-release device such as polymeric implant or mini-osmotic pump) can all be appropriate routes of administration.

The term “alleviating” means a reduction in the occurrence of a pain, of a headache, or of any symptom or cause of a condition or disorder. Thus, alleviating includes some reduction, significant reduction, near total reduction, and total reduction.

As used herein, the term “amino acid” includes the 22 amino acids that are proteinogenic amino acids and non-proteinogenic amino acids. The term “proteinogenic amino acid,” is used in the field of biochemistry to refer to the 22 amino acids that are incorporated into eukaryotic and/or prokaryotic proteins during translation, such as: (a) histidine (His; H); (b) isoleucine (Ile; I); (c) leucine (Leu; L); (d) Lysine (Lys; K); (e) methionine (Met; M); (f) phenylalanine (Phe; F); (g) threonine (Thr; T); (h) tryptophan (Trp; W); (i) valine (Val; V); (j) arginine (Arg; R); (k) cysteine (Cys; C); (l) glutamine (Gln; Q); (m) glycine (Gly; G); (n) proline (Pro; P); (o) serine (Ser; S); (p) tyrosine (Tyr; Y); (q) alanine (Ala; A); (r) asparagine (Asn; N); (s) aspartic acid (Asp; D); (t) glutamic acid (Glu; E); (u) selenocysteine (Sec; U); (v) pyrrolysine (Pyl; O). The term “non-proteinogenic amino acid” is used in the field of biochemistry to refer to naturally occurring and non-naturally occurring amino acids that are not proteinogenic amino acids, such as (1) citrulline (Cit); (2) cystine; (3) gamma-amino butyric acid (GABA); (4) ornithine (Orn); (5) theanine; (6) homocysteine (Hey); (7) thyroxine (Thx); and amino acid derivatives such as betaine; carnitine; carnosine creatine; hydroxytryptophan; hydroxyproline (Hyp); N-acetyl cysteine; S-Adenosyl methionine (SAM-e); taurine; tyramine, D-amino acids such as D-alanine (D-Ala); Norleucine (Nle); 4-hydroxyproline (HYP); 3,4-dehydro-L-proline (DHP); aminoheptanoic acid (AHP); (2R,5S)-5-phenyl-pyrrolidine-2-carboxylic acid (2PP); L-a-methylserine (MS); N-methylvaline (MV); 6-aminohexanoic acid (6-AHP); and 7-aminoheptanoic acid (7-AHP). Abbreviations for amino acid residues are used in keeping with standard polypeptide nomenclature delineated in IUPAC-IUB Biochem. Nom., J. Biol. Chem. 241: 527, 1966.

As used herein, “amino acid residue” means the individual amino acid units incorporated into a polypeptide. Amino acid residues are generally in the “L” isomeric form. However, residues in the “D” isomeric form can be substituted for any L-amino acid residue, as long as the desired functional property (e.g., substrate binding and/or cleavage of a substrate) is retained by the polypeptide. It should be noted that all amino-acid residue sequences are represented herein by formulae whose left and right orientation is in the direction of amino-terminus to carboxy-terminus. Furthermore, it should be noted that a dash at the beginning or end of an amino acid residue sequence indicates a peptide bond to a further sequence of one or more amino-acid residues.

As provided herein, amino acid substitutions are indicated by the amino acid residue being replaced and its amino acid position in the given amino acid sequence followed by the replacement amino acid. For example, an N240A amino acid modification relative to SEQ ID NO: 1 means that the asparagine residue at amino acid position 240 of SEQ ID NO: 1 is replaced with an alanine residue. When an amino acid substitution is indicated for polypeptide having a given percent sequence identity to a reference sequence, the indicated amino acid position is that of the reference sequence when the polypeptide is optimally aligned thereto. For example, an amino acid sequence having at least about 90% sequence identity to SEQ ID NO: 1 and having an N240A substitution means that when the amino acid sequence is optimally aligned to SEQ ID NO: 1, its amino acid residue that aligns with the asparagine residue at amino acid position 240 of SEQ ID NO: 1 is an alanine residue even though the alanine residue is not the 240th amino acid residue in the amino acid sequence itself.

The term “associated” refers to coincidence with the development or manifestation of a disease, condition or phenotype. Association may be due to, but is not limited to, genes responsible for housekeeping functions whose alteration can provide the foundation for a variety of diseases and conditions, those that are part of a pathway that is involved in a specific disease, condition or phenotype and those that indirectly contribute to the manifestation of a disease, condition or phenotype.

The term “biological activity” describes the beneficial or adverse effects of a drug on living matter. When a drug is a complex chemical mixture, this activity is exerted by the substance's active ingredient, but can be modified by the other constituents. Biological activity can be assessed as potency or as toxicity by an in vivo LD₅₀ or ED₅₀ assay, or through an in vitro assay such as, for example, cell-based potency assays as described in U.S. 20100203559 and U.S. 20100233802.

The compositions can comprise, consist essentially of, or consist of, the components disclosed.

The “binding pocket” as used herein refers to a region in the BoNT/A L-chain where amino acids are changed relative to the wild-type BoNT/A L-chain (SEQ ID NO: 1). By “binding pocket”, it is meant a region of the BoNT/A L-chain, which comprises one or more amino acids which are the contact points (e.g., via hydrogen-bond, salt bridge, and/or hydrophobic contact) for binding to the corresponding binding site of hSNAP-23, and/or which provide the space to accommodate other substrate amino acid residue(s) (e.g., by modification, such as by substitution) capable to bind hSNAP-23.

The term “binding to” as used herein encompasses “suitable for binding to.” For example, the BoNT/A L-chain protease binding pocket defined by amino acid residues E148, T307, A308 and Y312 of SEQ ID NO: 1 refers to a region of the BoNT/A L-chain protease comprising amino acids E148, T307, A308 and/or Y312, and/or mutants thereof that contribute to binding of a predicted binding site on hSNAP-23 (e.g., to the P182/D178 binding site of hSNAP-23).

The term “binding site” refers herein to a region of hSNAP-23, which comprises one or more amino acids that can be bound by the corresponding BoNT/A L-chain binding pocket. For example, the “P182/D178” binding site of hSNAP-23 comprises the amino acids P182 and/or D178 of hSNAP-23.

The terms “botulinum toxin” or “botulinum neurotoxin” can be used herein interchangeably, and refer to a neurotoxin produced by Clostridium botulinum, as well as a botulinum toxin or neurotoxin fragments, functional fragments, variants, functional variants, or chimeras thereof made recombinantly by a non-Clostridial species. The terms “botulinum toxin” and “botulinum neurotoxin”, as used herein, encompass botulinum toxin types A, B, C₁, D, E, F and G and mosaics (including but not limited to CD, DC, FA) and non-clostridial BoNT-like encoding sequences (including but not limited to BoNT/X, BoNT/Wo, BoNT/En (eBoNT/J), Cp1, PMP1); and/or their subtypes and any other types of subtypes thereof, or any re-engineered proteins, analogs, derivatives, homologs, parts, sub-parts, variants, or versions, in each case, of any of the foregoing. Further “botulinum toxin” or “botulinum neurotoxin”, as used herein, also encompasses a botulinum toxin complex, (for example, the 300, 600 and 900 kDa complexes), as well as the neurotoxic component of the botulinum toxin (150 kDa) that is unassociated with the complex proteins.

The terms “botulinum neurotoxin protease variant having the amino acid sequence of SEQ ID NO: 27”, “protease variant having SEQ ID NO: 27”, “protease variant of SEQ ID NO: 27”, “reference protease variant of SEQ ID NO: 27”, “SEQ ID NO: 27”, and “qmLC/A” are used interchangeably and refer to a quadruple mutant (“qm”) of the light chain of botulinum neurotoxin type A (BoNT/A) with the E148Y, K166F, S254A, and G305D substitutions as disclosed in WO2019/145577, which is incorporated herein by reference.

The term “clostridial toxin” refers to any toxin produced by a Clostridial toxin strain that can execute the overall cellular mechanism, whereby a Clostridial toxin intoxicates a cell and encompasses the binding of a Clostridial toxin to a low or high affinity Clostridial toxin receptor, the internalization of the toxin/receptor complex, the translocation of the Clostridial toxin light chain into the cytoplasm and the enzymatic modification of a Clostridial toxin substrate. Non-limiting examples of Clostridial toxins include a Botulinum toxin like BoNT/A, a BoNT/B, a BoNT/C₁, a BoNT/CD, BoNT/D, a BoNT/DC a BoNT/E, a BoNT/F, a BoNT/FA, a BoNT/G, a BoNT/X, an Enterococcus faecium toxin (BoNT/En also called eBoNT/J), a Weissella oryzae toxin (BoNT/Wo), a Chryseobacterium piperi toxin (Cp1), a Paraclostridium bifermentans toxin (PMP1), a Tetanus toxin (TeNT), a Baratii toxin (BaNT), and a Butyricum toxin (BuNT). The BoNT/C2 cytotoxin and BoNT/C3 cytotoxin, not being neurotoxins, are excluded from the term “Clostridial toxin.” A Clostridial toxin disclosed herein includes, without limitation, naturally occurring Clostridial toxin variants, such as, e.g., Clostridial toxin isoforms and Clostridial toxin subtypes; non-naturally occurring Clostridial toxin variants, such as, e.g., conservative Clostridial toxin variants, non-conservative Clostridial toxin variants, Clostridial toxin chimeric variants and active Clostridial toxin fragments thereof, or any combination thereof. A Clostridial toxin disclosed herein also includes a Clostridial toxin complex. As used herein, the term “Clostridial toxin complex” refers to a complex comprising a Clostridial toxin and non-toxin associated proteins (NAPs), such as, e.g., a Botulinum toxin complex, a Tetanus toxin complex, a Baratii toxin complex, and a Butyricum toxin complex. Non-limiting examples of Clostridial toxin complexes include those produced by a Clostridium botulinum, such as, e.g., a 900-kDa BoNT/A complex, a 500-kDa BoNT/A complex, a 300-kDa BoNT/A complex, a 500-kDa BoNT/B complex, a 500-kDa BoNT/C₁ complex, a 500-kDa BoNT/D complex, a 300-kDa BoNT/D complex, a 300-kDa BoNT/E complex, and a 300-kDa BoNT/F complex.

The phrase “Clostridial toxin active ingredient” refers to a molecule that contains any part of a clostridial toxin that exerts an effect upon or after administration to a subject or patient. As used herein, the term “clostridial toxin active ingredient” encompasses a Clostridial toxin complex comprising the approximately 150-kDa Clostridial toxin and other proteins collectively called non-toxin associated proteins (NAPs), the approximately 150-kDa Clostridial toxin alone, or a modified Clostridial toxin, such as, e.g., a re-targeted Clostridial toxins.

As used herein, the term “culture,” refers to any sample or specimen that is suspected of containing one or more microorganisms or cells. “Pure cultures” are cultures in which the cells or organisms are only of a particular species or genus. This is in contrast to “mixed cultures,” wherein more than one genus or species of microorganism or cell are present.

“Detect” and “detection” have their standard meaning, and are intended to encompass detection, measurement and/or characterization of a selected protein or protein activity. For example, enzyme activity may be “detected” in the course of detecting, screening for, or characterizing inhibitors, activators, and modulators of the protein.

A “domain” as used herein, is a portion of a protein that has a tertiary structure. The domain may be connected to other domains in the complete protein by short flexible regions of polypeptide. Alternatively, the domain may represent a functional portion.

“Effective amount” as applied to the biologically active ingredient means that amount of the ingredient which is generally sufficient to effect a desired change in the subject. For example, where the desired effect is a reduction a symptom, an effective amount of the ingredient is that amount which causes at least a substantial reduction of the symptom, and without resulting in significant toxicity.

The term “exemplary” as used herein has the meaning of illustrative, serving as an example. For example, “exemplary amino acid substitutions” as used herein means examples of amino acid substitutions; or “exemplary mutants” means examples of mutants.

“Heavy chain” means the heavy chain of a botulinum neurotoxin. It has a molecular weight of about 100 kDa and can be referred to as the H chain, HC, or as H.

The term “light chain” means the light chain of a clostridial neurotoxin. It has a molecular weight of about 50 kDa, and can be referred to as the L chain, LC, L, or as the proteolytic domain (amino acid sequence) of a botulinum neurotoxin.

The phrase “improved enzyme property” or “increased enzyme property” refers to a functional property of a polypeptide that can be measured under suitable conditions and which exhibits improvement as compared to the same property of a reference polypeptide. For the LC/A protease variant polypeptides described herein, the comparison is generally made to the wild-type LC/A protease enzyme, although the reference polypeptide can be another evolved or improved LC/A protease polypeptide. Enzyme properties for which improvement is desirable include, but are not limited to, enzymatic activity (which can be expressed in terms of percent conversion of the substrate), substrate specificity, substrate catalysis or cleavage, substrate binding, thermo stability, solvent stability, pH activity profile, concentration of sodium chloride and other physiologically relevant salts, cofactor requirements, refractoriness to inhibitors (e.g., inhibition by interfering substances, substrates, or products), and stereospecificity (including enantiospecificity).

“Increased enzymatic activity” or “increased activity” refers to an improved property of an engineered, evolved, or a variant enzyme, which can be represented by an increase in enzyme activity (e.g., product produced/time/weight protein) or an increase in percent conversion of the substrate to the product (e.g., percent conversion of starting amount of substrate to product in a specified time period using a specified amount of an LC/A or LC/E protease) as compared to a reference enzyme. Exemplary methods to determine enzyme activity are provided in the Examples. Any property relating to enzyme activity may be affected, including the classical enzyme properties of Km, Vmax or kcat, changes of which can lead to increased enzymatic activity. Comparisons of enzyme activities are made using a defined preparation of enzyme, a defined assay under a set condition, and one or more defined substrates, as further described in detail herein. Generally, when enzymes in cell lysates are compared, the numbers of cells and the amount of protein assayed are determined as well as use of identical expression systems and identical host cells to minimize variations in amount of enzyme produced by the host cells and present in the lysates.

The term “isolated” as used herein means a nucleic acid sequence or a polypeptide sequence that is separated from the wild or native sequence in which it naturally occurs or is in an environment different from that in which the sequence naturally occurs.

The term “isolated polypeptide” refers to a polypeptide that is substantially separated from other contaminants that naturally accompany it, e.g., protein, lipids, and polynucleotides. The term embraces polypeptides that have been removed or purified from their naturally occurring environment or expression system (e.g., host cell or in vitro synthesis). The evolved or improved LC/A protease enzymes may be present within a cell, present in the cellular medium, or prepared in various forms, such as lysates or isolated preparations. As such, in some embodiments, the evolved or improved LC/A protease polypeptides can be an isolated polypeptide.

The phrase “local administration” means direct administration of a pharmaceutical at or to the vicinity of a site on or within an animal body, at which site a biological effect of the pharmaceutical is desired, such as via, for example, intramuscular or intra- or subdermal injection or topical administration. Local administration excludes systemic routes of administration, such as intravenous or oral administration. Topical administration is a type of local administration in which a pharmaceutical agent is applied to a patient's skin.

The terms “modified botulinum toxin”, “modified botulinum neurotoxin”, “modified botulinum neurotoxin protein”, “modified botulinum neurotoxin protein variant”, “modified botulinum neurotoxin protease”, and “modified botulinum neurotoxin protease variants” are used interchangeably, and refer to a botulinum toxin that has had at least one of its amino acids deleted, modified, or replaced, as compared to a native botulinum toxin. The term “modified botulinum toxin” and the like encompass fragments, functional fragments, variants, functional variants, or chimeras of a native botulinum toxin, as well as a modified botulinum toxin and fragments, functional fragments, variants, functional variants, or chimeras thereof made recombinant by a non-Clostridial species. It is further understood that, fragments, functional fragments, variants, functional variants, or chimeras of a modified botulinum toxin and the like encompass nucleic acid and/or amino acid sequences having at least 70% (for example, at least 80%, or at least 85%, or at least 90%, or at least 95%, or at least 97%, or at least 98%, or at least 99%, or at least about or about 99.1%, 99.2%, 99.3%, 99.4%, 99.5%, 99.6%, 99.7%, 99.8%, or 99.9%) sequence identity to the corresponding native botulinum toxin.

An engineered or modified botulinum toxin can be a non-naturally occurring botulinum toxin. Additionally, the engineered or modified botulinum toxin can be a recombinantly produced neurotoxin, or a derivative or fragment of a recombinantly made neurotoxin. An engineered or modified botulinum toxin retains at least one or more of the biological activities of the native botulinum toxin, such as, the ability to bind to a botulinum toxin receptor, the ability to inhibit neurotransmitter release from a neuron, ability to inhibit release and/or transfer of vesicles in neuronal or non-neuronal cells, the ability to cleave non-neuronal SNARE proteins, and/or the ability to treat conditions associated with SNARE proteins. One example of an engineered or modified botulinum toxin is a botulinum toxin that has a light chain from one botulinum toxin serotype (such as serotype A), and a heavy chain from a different botulinum toxin serotype (such as serotype B). Another example of an engineered or modified botulinum toxin is a botulinum toxin coupled to a neurotransmitter, such as substance P.

The terms “modification”, “modified”, “change” or “mutation” can be used herein interchangeably, and refer to the alteration in the amino acid sequence compared to that of a protein of reference, e.g., as used herein relative to the wild-type BoNT/A L-chain (SEQ ID NO: 1). The exemplary BoNT/A L-chain amino acid sequence illustrated herein as SEQ ID NO: 1 is 448 amino acid residues in length and ends with K448, where the numbering includes the initiator methionine as translated. It is understood that K438 of SEQ ID NO: 1 is the first lysine amino acid residue of the activation loop, whereas K448 of SEQ ID NO: 1 is the last lysine amino acid residue of the activation loop. In some embodiments, the activation loop of BoNT/A protease comprises the exemplary amino acid sequence of SEQ ID NO: 32 and the activation loop is formed by the two cysteine residues (e.g., C1 and C25 of SEQ ID NO: 32) that forms a disulfide bond. In some embodiments, the BoNT/A protease is cleaved at both lysine (K) residues, e.g., at K338 and K448 of SEQ ID NO: 1, which removes the ten amino acid residues after K338. In such some embodiments, K338 most likely represents the C-terminal end of the L-chain after proteolytic cleavage of the activation loop. Thus, in some embodiments, the sequence encompassing amino acids 1-438 of SEQ ID NO: 1 represents the activated form of a wild-type BoNT/A L-chain (including the initiator methionine). In some embodiments, the sequence encompassing amino acids 1-438 of SEQ ID NO: 1 may represent the most naturally activated form of a wild-type BoNT/A L-chain with the initiator methionine included.

In this regard, it should be understood that prior to any post-translational modification and proteolytic activation, a wild-type BoNT/A L-chain gene product (e.g., a RNA or protein) is about 448 amino acid residues in length, which includes a short C-terminal extension of activation loop amino acid residues beyond the L-chain cysteine (C) that forms the interchain disulfide bridge to the H-chain. In some instances, the sequence encompassing amino acids 1-438 sequence of the BoNT/A1 L-chain is often isolated from the native protein. In some embodiments, BoNT/A L-chains of alternate lengths may be isolated following other native, incomplete or alternate proteolytic activation after other lysine residues in the activation loop. For example, in some embodiments, a native, incomplete or alternate proteolytic processing following K440 would yield a BoNT/A L-chain sequence of 1-440 amino acids. In some embodiments, a native, incomplete or alternate proteolytic processing following K444 would yield a BoNT/A L-chain sequence of 1-444 amino acids. In yet some embodiments, a native, incomplete or alternate proteolytic processing following K448 would yield a BoNT/A L-chain sequence of 1-448 amino acids. It is understood that while these are exemplary embodiments of native proteolytic processing of BoNT/A proteases (e.g., native complete as well as native incomplete or alternate proteolytic processing), engineered nicking of other amino acid residues at non-native activation cleavage site(s) in the activation loop in a botulinum neurotoxin protein and fragments thereof will provide alternate BoNT/A L-chain lengths as describe in detail below. It is understood that proteolytic nicking at various native as well at non-native activation cleavage site(s) are also contemplated for BoNT/E proteases. In some embodiments, alternate sequences of BoNT/A and BoNT/E activation loops are also contemplated for engineered nicking at non-native activation cleavage site(s). In some embodiments, the amino acid sequence of any one of SEQ ID NO: 5-23, 27, 28 or 31 may include at its C-terminal end, part or all of the amino acid residue(s) of an alternate activation loop, for example, for cleavage by alternate activation proteases.

Further, while the exemplary wild-type BoNT/A L-chain (SEQ ID NO: 1) and the botulinum neurotoxin protein sequences disclosed herein (see, for example, SEQ ID NOS: 5-23, 27, 28, and 31) include the initiator methionine (M) amino acid as translated, it is understood that the initiator methionine may or may not be removed post-translationally depending on the expression system and expression conditions used for expression and purification of such proteins. For example, in a bacterial expression system, post-translational processing of a gene product according to various embodiments may involve removal of the initiating methionine, formation of disulfide bridges, and/or limited proteolysis (nicking) by bacterial protease(s).

The term “mutation” means a structural modification of a naturally occurring protein or nucleic acid sequence. For example, in the case of nucleic acid mutations, a mutation can be a deletion, addition or substitution of one or more nucleotides in the DNA sequence. In the case of a protein sequence mutation, the mutation can be a deletion, addition, insertion, or substitution of one or more amino acids in a protein sequence. A “conservative” amino acid substitution, as used herein, generally refer to substitution of one amino acid residue with another amino acid residue from within a recognized group which changes the structure of the peptide but biological activity of the peptide is substantially retained. Conservatively substituted amino acids can be identified using a variety of well know methods, such as a blocks substitution matrix (BLOSUM), e.g., BLOSUM62 matrix. BLOSUM is a substitution matrix used for sequence alignment of proteins, wherein an alignment score is used to map out relationship between evolutionarily divergent protein sequences. They are based on local alignments. For instance, a BLOSUM62 substitution matrix can be found in NCBI.NLM.NIH.GOV/class/fieldguide/BLOSUM62.txt, which is incorporated by reference. Exemplary amino acid substitutions can be found in Table A.

TABLE A
Exemplary amino acid substitutions

| Amino Acid | Exemplary Conservative Substitutions |
|---|---|
| Ala | Ser, Gly, Cys |
| Arg | Lys, Gln, Met, Ile |
| Asn | Gln, His, Glu, Asp |
| Asp | Glu, Asn, Gln |
| Cys | Ser, Met, Thr |
| Gln | Asn, Lys, Glu, Asp |
| Glu | Asp, Asn, Gln |
| Gly | Pro, Ala |
| His | Asn, Gln |
| Ile | Leu, Val, Met |
| Leu | Ile, Val, Met |
| Lys | Arg, Gln, Met, Ile |
| Met | Leu, Ile, Val |
| Phe | Met, Leu, Tyr, Trp, His |
| Ser | Thr, Met, Cys |
| Thr | Ser, Met, Val |
| Trp | Tyr, Phe |
| Tyr | Trp, Phe, His |
| Val | Ile, Leu, Met |

For example, a specific amino acid in a protein sequence can be substituted for another amino acid, for example, an amino acid selected from a group which includes the amino acids alanine, asparagine, cysteine, aspartic acid, glutamic acid, phenylalanine, glycine, histidine, isoleucine, lysine, leucine, methionine, proline, glutamine, arginine, serine, threonine, valine, tryptophan, tyrosine or any other natural or non-naturally occurring amino acid or chemically modified amino acids. Mutations to a protein sequence can be the result of mutations to DNA sequences that when transcribed, and the resulting mRNA translated, produce the mutated protein sequence. Mutations to a protein sequence can also be created by fusing a peptide sequence containing the desired mutation to a desired protein sequence.

The terms “nucleic acid molecule” and “polynucleotide” are used herein to include a polymeric form of nucleotides of any length, either ribonucleotides or deoxyribonucleotides. This term refers only to the primary structure of the molecule. Thus, the term includes triple-, double- and single-stranded DNA, as well as triple-, double- and single-stranded RNA. It also includes modifications, such as by methylation and/or by capping, and unmodified forms of the polynucleotide. More particularly, the terms “polynucleotide” and “nucleic acid molecule” include polydeoxyribonucleotides (containing 2-deoxy-D-ribose), polyribonucleotides (containing D-ribose), any other type of polynucleotide which is an N- or C-glycoside of a purine or pyrimidine base, and other nonnucleotidic backbones, for example, polyamide (e.g., peptide nucleic acids (PNAs)) and polymorpholino polymers (commercially available from the Anti-Virals, Inc., Corvallis, OR, USA, as NEUGENE), and other synthetic sequence-specific nucleic acid polymers provided that the polymers contain nucleobases in a configuration which allows for base pairing and base stacking, such as is found in DNA and RNA. There is no intended distinction in length between the terms “nucleic acid molecule” and “polynucleotide.”

As used herein, the term “nucleotide” refers to molecules that, when joined, make up the individual structural units of the nucleic acids RNA and DNA. A nucleotide is composed of a nucleobase (nitrogenous base), a five-carbon sugar (either ribose or 2-deoxyribose), and one phosphate group. “Nucleic acids” as used herein are polymeric macromolecules made from nucleotide monomers. In DNA, the purine bases are adenine (A) and guanine (G), while the pyrimidines are thymine (T) and cytosine (C). RNA uses uracil (U) in place of thymine (T).

As used herein, a “nucleic acid,” “polynucleotide,” or “oligonucleotide” can be a polymeric form of nucleotides of any length, can be DNA or RNA, and can be single- or double-stranded. Nucleic acids can include promoters or other regulatory sequences. Oligonucleotides can be prepared by synthetic means. Nucleic acids include segments of DNA, or their complements spanning or flanking any one of the polymorphic sites. The segments can be between 5 and 1000 contiguous bases and can range from a lower limit of 5, 20, 50, 100, 200, 300, 500, 700 or 1000 nucleotides to an upper limit of 500, 1000, 2000, 5000, or 10000 nucleotides (where the upper limit is greater than the lower limit). Nucleic acids between 5-20, 50-100, 50-200, 100-200, 120-300, 150-300, 100-500, 200-500, or 200-1000 bases are common. A reference to the sequence of one strand of a double-stranded nucleic acid defines the complementary sequence and except where otherwise clear from context, a reference to one strand of a nucleic acid also refers to its complement. Complementation can occur in any manner, e.g., DNA=DNA; DNA=RNA; RNA=DNA; RNA=RNA, wherein, in each case, the “=” indicates complementation. Complementation can occur between two strands or a single strand of the same or different molecule.

The term “protease” used herein refers to an enzyme, which is capable of hydrolytically cleaving proteins and/or peptides. A protease is more particularly a botulinum neurotoxin (BoNT) light-chain (L-chain) protease, e.g., a protease (also described as “proteolytic domain” or “protease domain”) derived from botulinum neurotoxin, in particular from botulinum neurotoxin A (BoNT/A) and botulinum neurotoxin E (BoNT/E).

The terms “protein,” “polypeptide,” “oligopeptide,” and “peptide” are used interchangeably to denote a polymer of at least two amino acids covalently linked by an amide bond, regardless of length or post-translational modification (e.g., glycosylation, phosphorylation, lipidation, myristilation, ubiquitination, etc.). Included within this definition are D- and L-amino acids, and mixtures of D- and L-amino acids.

The term “SNAP-23” or “SNAP23” (synaptosomal-associated protein 23) designates herein a SNARE protein, which is capable of binding to various other SNARE proteins and of forming a high affinity complex with these proteins in a cell, for example in a non-neuronal cell, thereby regulating intracellular cell membrane fusion in said cell. “hSNAP-23” refers more particularly to human SNAP-23, or to the protein of sequence SEQ ID NO: 2.

The term “SNAP-25” or “SNAP25” (synaptosomal-associated protein 25) designates herein a SNARE protein, which is capable of binding to various other SNARE proteins and of forming a high affinity complex in a cell, such as a neuronal cell, thereby regulating intracellular cell membrane fusion in said cell. “hSNAP-25” refers more particularly to human SNAP-25, or to the protein of sequence SEQ ID NO: 3.

The term “SNAP-29” or “SNAP29” (synaptosomal-associated protein 29) designates herein a SNARE protein, which is capable of binding to various other SNARE proteins and of forming a high affinity complex in a cell, such as a cancer cell, thereby regulating intracellular cell membrane fusion in said cell. “hSNAP-29” refers more particularly to human SNAP-29, or to the protein of sequence SEQ ID NO: 4.

The term “sequence identity” between amino acid or nucleic acid sequences means amino acid or nucleic acid sequence identity in two or more aligned sequences aligned using a sequence alignment program. Nucleic acid or amino acid sequence identity in two or more sequences can be determined by comparing a position in each of the sequences which may be aligned for the purposes of comparison. When a position in the compared sequences is occupied by the same nucleotide or amino acid, then the sequences are identical at that position. A degree of identity between amino acid sequences is a function of the number of identical amino acid sequences that are shared between these sequences. A degree of sequence identity between nucleic acids is a function of the number of identical nucleotides at positions shared by these sequences. Exemplary computer programs which can be used to determine identity between two sequences include, but are not limited to, the suite of BLAST programs, e.g., BLASTN, BLASTX, and TBLASTX, BLASTP and TBLASTN, publicly available on the Internet at (ncbi.nlm.gov/BLAST/). See, also, Altschul, S. F. et al., 1990 and Altschul, S. F. et al., 1997.

The phrase “percentage of sequence identity” and “percentage homology” are used interchangeably herein to refer to comparisons among polynucleotides (nucleic acid sequences) and polypeptides (amino acid sequences). To determine the “percentage of sequence identity” between two amino acid sequences or two nucleic acid sequences, the sequences are aligned for optimal comparison. For example, gaps can be introduced in the sequence of a first amino acid sequence or a first nucleic acid sequence for optimal alignment with the second amino acid sequence or second nucleic acid sequence. The amino acid residues or nucleotides at corresponding amino acid positions or nucleotide positions are then compared. When a position in the first sequence is occupied by the same amino acid residue or nucleotide as the corresponding position in the second sequence, the molecules are identical at that position.

The phrase “% sequence identity”, “percent identity”, or “percent identical”, refers to the level of nucleic acid or amino acid sequence identity between two or more aligned sequences, when aligned using a sequence alignment program. For example, 70% homology means the same thing as 70% sequence identity determined by a defined algorithm, and accordingly a homologue of a given sequence has greater than 70% sequence identity over a length of the given sequence. Exemplary levels of sequence identity include, but are not limited to 70%, 75% 80%, 85%, 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or 100% sequence identity to a given sequence.

The percentage (%) of identity between the two sequences is a function of the number of identical positions shared by the sequences. Hence, the percentage of identity can be calculated by multiplying the number of identical positions by 100 and dividing by the length of the aligned region (overlapping positions), including gaps (only internal gaps, not the gaps at the sequence ends). In this comparison, the sequences can be of the same length, or may be of different lengths. Identity scoring only counts perfect matches and does not consider the degree of similarity of amino acids to one another.

Optimal alignment of sequences may be conducted by a global homology alignment algorithm should the alignment be performed using sequences of the same or similar length, such as by the algorithm described by Needleman and Wunsch (Journal of Molecular Biology; 1970, 48 (3): 443-53), by computerized implementations of this algorithm (e.g., using the DNASTAR® Lasergene software), or by visual inspection. Alternatively, should the alignment be performed using sequences of distinct length (e.g. the amino acid sequence of the light-chain versus the entire amino acid sequence of a naturally occurring botulinum neurotoxin), the optimal alignment of sequences can be conducted by a local homology alignment algorithm, such as by the algorithm described by Smith and Waterson (Journal of Molecular Biology; 1981, 147: 195-197), by computerized implementations of this algorithm (e.g., using the DNASTAR® Lasergene software), or by visual inspection. The best alignment (e.g., resulting in the highest percentage of identity between the compared sequences) generated by the various methods is selected. Examples of global and local homology alignment algorithms include, without limitation, ClustalV (global alignment), ClustalW (local alignment) and BLAST (local alignment).

As used herein, “substantially identical” in reference to an amino sequence or nucleotide sequence means that a candidate sequence is at least 70% sequence identity to a reference sequence over a given comparison window (e.g., 250 amino acids). Thus, substantially similar sequences include those having, for example, at least 80% sequence identity, at least 85%, 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, or 99.9% sequence identity. Two sequences that are identical to each other are also substantially similar. The comparison window or the length of comparison sequence will generally be at least the length of the protein fragment or domain of interest, or of the full protein. Sequence identity is calculated based on the reference sequence and algorithms for sequence analysis may be used for the sequence identity calculations. Thus, to determine percent sequence identity of two amino acid sequences, the sequences are aligned for optimal comparison purposes (e.g., gaps can be introduced in the sequence of one polypeptide for optimal alignment with the other polypeptide). The amino acid residues at corresponding amino acid positions are then compared. When a position in one sequence is occupied by the same amino acid residue as the corresponding position in the other sequence, then the molecules are identical at that position. The percent sequence identity between the two sequences is a function of the number of identical positions shared by the sequences (e.g., percent sequence identity=numbers of identical positions/total numbers of positions×100). Percent sequence identity between two polypeptide sequences can be determined using the Vector NTI® software package (Invitrogen Corp., Carlsbad, CA). A gap opening penalty of 10 and a gap extension penalty of 0.1 are used for determining the percent identity of two polypeptides. All other parameters are set at the default.

By reserving the right to proviso out or exclude any individual members of any such group, including any sub-ranges or combinations of sub-ranges within the group, that can be claimed according to a range or in any similar manner, less than the full measure can be claimed for any reason. Further, by reserving the right to proviso out or exclude any individual substituents, analogs, compounds, ligands, structures, or groups thereof, or any members of a claimed group, less than the full measure can be claimed for any reason.

Various patents, patent applications and publications are referenced. The disclosures of these patents, patent applications and publications in their entireties are incorporated by reference in order to more fully describe the state of the art as known to those skilled therein as of the date. This disclosure will govern in the instance that there is any inconsistency between the patents, patent applications and publications cited and this disclosure.

### Engineered Mutant Botulinum Neurotoxin Protease Domains and Proteins

Botulinum neurotoxin proteins and fragments thereof are provided. The botulinum neurotoxin protein or fragment thereof is, in some embodiments, a botulinum neurotoxin (BoNT) comprising an amino acid sequence that is modified, relative to the L-chain (LC) protease of botulinum neurotoxin serotype A (SEQ ID NO: 1), wherein the modified amino acid sequence comprises at least 2 of the amino acid substitutions set forth in Table 1 and one or more of the amino acid substitutions set forth in Table 3. That is, in some embodiments, a botulinum neurotoxin protein is provided that comprises an amino acid sequence with at least about 90%, 91%, 92%, 93%, 94%, 95%, 96%, 97%, 98% or 99% sequence identity to SEQ ID NO: 1 and wherein the amino acid sequence has at least two amino acid positions with an amino acid modification set forth in Table 1 and at least one amino acid position with one of the amino acid modifications set forth in Table 3.

The botulinum neurotoxin protein or fragment thereof is, in some embodiments, a botulinum neurotoxin comprising an amino acid sequence that is modified, relative to the L-chain protease of botulinum neurotoxin serotype A (SEQ ID NO: 1), in that it comprises one of the sets of amino acid substitutions set forth in Table 2 and one or more of the amino acid substitutions set forth in Table 3. That is, in some embodiments, a botulinum neurotoxin protein is provided that comprises an amino acid sequence with at least about 90%, 91%, 92%, 93%, 94%, 95%, 96%, 97%, 98% or 99% sequence identity to SEQ ID NO: 1 and wherein the amino acid sequence has (i) one set of amino acid modifications set forth in Table 2 and (ii) one or more amino acid positions with an amino acid modification set forth in Table 3.

In some embodiments, the botulinum neurotoxin protein or fragment thereof is a botulinum neurotoxin comprising an amino acid sequence that is modified, relative to the L-chain protease of botulinum neurotoxin serotype A (SEQ ID NO: 1), in that it comprises one or more of the amino acid substitutions set forth in Table 3. That is, in some embodiments, a botulinum neurotoxin protein is provided that comprises an amino acid sequence with at least about 90%, 91%, 92%, 93%, 94%, 95%, 96%, 97%, 98% or 99% sequence identity to SEQ ID NO: 1 and wherein the amino acid sequence has one or more amino acid positions with one or more of the amino acid substitutions set forth in Table 3.

TABLE 1
Amino acid modifications relative to wild-type
BoNT/A light chain (SEQ ID NO: 1).
Modified Residues

Q29A
S143 D, E or Q
E148N or Y
K166V, F, L or I
Y251 E or D
S254A
L256 E or D
V304 D or E
G305 D or E
T307 I or L
A308 L, P, N, T or I
Y312 K, V, M or L

TABLE 2
Amino acid modifications relative to wild-type
BoNT/A light chain (SEQ ID NO: 1).
Modified Residues

Y312K, E148Y
L256E, V258P
E148Y, K166F
T307I, A308P, Y312V
T307F, A308N, Y312V
E148N, T307I, A308P, Y312V
E148Y, T307F, A308N, Y312L
E148Y, T307I, A308P, Y312V
E148Y, T307L, A308T, Y312M
E148Y, K166F, G305D
E148Y, K166F, S254A, G305D

TABLE 3
One or more of the following modifications relative to
BoNT/A light chain (SEQ ID NO: 1), optionally with
one or more of the modifications in Tables 1 or 2.
Modified Residues

N26S
A27 L or R
Q29 R or S
N53 H or R
E55 I, V or N
E56I
Q162R
E201D
D203V
N240 A or S
S254 L or M
K364R
Y387N

In some embodiments, the botulinum neurotoxin protein or fragment thereof is a botulinum neurotoxin comprising an amino acid sequence that, compared to the L-chain protease of botulinum neurotoxin serotype A (SEQ ID NO: 1), comprises at least two of the amino acid substitutions set forth in Table 1 and one or more of the amino acid substitutions or one of the sets of amino acid substitutions set forth in Table 4. That is, a botulinum neurotoxin protein is provided that comprises an amino acid sequence with at least about 90%, 91%, 92%, 93%, 94%, 95%, 96%, 97%, 98% or 99% sequence identity to SEQ ID NO: 1 and wherein the amino acid sequence has (i) at least two amino acid positions modified by an amino acid modification set forth in Table 1 and (ii) one or more of the amino acid modifications set forth in Table 4 or one set of amino acid modifications set forth in Table 4.

In some embodiments, the botulinum neurotoxin protein or fragment thereof is a botulinum neurotoxin comprising an amino acid sequence that, compared to the L-chain protease of botulinum neurotoxin serotype A (SEQ ID NO: 1), comprises one of the sets of amino acid substitutions set forth in Table 2 and one or more of the amino acid substitutions or one of the sets of amino acid substitutions set forth in Table 4. That is, a botulinum neurotoxin protein is provided that comprises an amino acid sequence with at least about 90%, 91%, 92%, 93%, 94%, 95%, 96%, 97%, 98% or 99% sequence identity to SEQ ID NO: 1 and wherein the amino acid sequence has (i) at least one set of amino acid modifications set forth in Table 2 and (ii) one or more of the amino acid modifications set forth in Table 4 or one set of amino acid modifications set forth in Table 4.

In some embodiments, the botulinum neurotoxin protein or fragment thereof is a botulinum neurotoxin comprising an amino acid sequence having, when compared to the L-chain protease of botulinum neurotoxin serotype A (SEQ ID NO: 1), at least 2 of the following amino acid substitutions: E148Y, K166F, S254A, G305D, and one or more of the amino acid substitutions set forth in Table 3. That is, a botulinum neurotoxin protein is provided that comprises an amino acid sequence with at least about 90%, 91%, 92%, 93%, 94%, 95%, 96%, 97%, 98% or 99% sequence identity to SEQ ID NO: 1 and wherein the amino acid sequence has (i) at least 2 of the following amino acid substitutions: E148Y, K166F, S254A, G305D, and (ii) one or more of the amino acid substitutions set forth in Table 3.

In some embodiments, the botulinum neurotoxin protein or fragment thereof is a botulinum neurotoxin comprising an amino acid sequence having, relative to the L-chain protease of botulinum neurotoxin serotype A (SEQ ID NO: 1), at least 2 of the following amino acid substitutions: E148Y, K166F, S254A, G305D, and one or more of the amino acid substitutions or one of the sets of amino acid substitutions set forth in Table 4. That is, a botulinum neurotoxin protein is provided that comprises an amino acid sequence with at least about 90%, 91%, 92%, 93%, 94%, 95%, 96%, 97%, 98% or 99% sequence identity to SEQ ID NO: 1 and wherein the amino acid sequence has (i) at least 2 of the following amino acid substitutions: E148Y, K166F, S254A, G305D, and (ii) one or more of the amino acid substitutions set forth in Table 4 or one set of amino acid substitutions set forth in Table 4.

TABLE 4
Exemplary mutants: Amino acid modifications relative to
wild-type BONT/A light chain (SEQ ID NO: 1).
Modified Residues

E201D, D203V
N240S
K364R, Y387N
N240A
N26S, N240A
N26S, E55V, N240A
N26S, E55I, N240A
N26S, Q29R, E55V, N240A
N26S, Q29S, E55V, N240A
N26S, Q29R, N53R, E55V, N240A
N26S, Q29R, N53R, E55V, N240A, S254L
N26S, Q29R, N53R, E55V, N240A, S254L
N26S, Q29R, N53H, E55V, N240A, S254L
N26S, Q162R, N240A
N26S, E55N, N240A
N26S, A27L, Q29R, N53R, E55V, N240A
N26S, A27R, Q29R, N53R, E55V, N240A
N26S, Q29R, N53R, E55V, E56I, N240A
N26S, Q29R, N53R, E55V, N240A, S254M

TABLE 5
Exemplary mutants: Amino acid modifications relative
to wild-type BONT/A light chain (SEQ ID NO: 1).

| SEQ ID NO: | Modified Residues |
|---|---|
| 5 | E148Y, K166F, N240S, S254A, G305D |
| 6 | E148Y, K166F, N240A, S254A, G305D |
| 7 | N26S, E148Y, K166F, N240A, S254A, G305D |
| 8 | N26S, E55V, E148Y, K166F, N240A, S254A, G305D |
| 9 | N26S, Q29R, E55V, E148Y, K166F, N240A, S254A, G305D |
| 10 | N26S, Q29R, N53R, E55V, E148Y, K166F, N240A, S254A, G305D |
| 11 | N26S, Q29R, N53R, E55V, E148Y, K166F, N240A, S254L, G305D |
| 12 | N26S, Q29R, N53R, E55V, E148Y, K166F, N240A, S254L |
| 13 | N26S, Q29R, N53H, E55V, E148Y, K166F, N240A, S254L |

TABLE 6
Exemplary amino acid modifications relative to
wild-type BoNT/A light chain (SEQ ID NO: 1):

| SEQ ID NO: | Modified Residues |
|---|---|
| 14 | E148Y, K166F, E201D, D203V, S254A, G305D |
| 15 | E148Y, K166F, S254A, G305D, K364R, Y387N |
| 16 | N26S, E148Y, Q162R, K166F, N240A, S254A, G305D |
| 17 | N26S, E55N, E148Y, K166F, N240A, S254A, G305D |
| 18 | N26S, E55I, E148Y, K166F, N240A, S254A, G305D |
| 19 | N26S, Q29S, E55V, E148Y, K166F, N240A, S254A, G305D |
| 20 | N26S, A27L, Q29R, N53R, E55V, E148Y, K166F, N240A, S254A, G305D |
| 21 | N26S, A27R, Q29R, N53R, E55V, E148Y, K166F, N240A, S254A, G305D |
| 22 | N26S, Q29R, N53R, E55V, E56I, E148Y, K166F, N240A, S254A, G305D |
| 23 | N26S, Q29R, N53R, E55V, E148Y, K166F, N240A, S254M, G305D |

In some embodiments, a botulinum neurotoxin protein or fragment thereof, comprising an amino acid sequence that, relative to the L-chain protease of botulinum neurotoxin serotype A (SEQ ID NO: 1), comprises one of the sets of amino acid substitutions set forth in Table 5. A botulinum neurotoxin protein is provided that comprises an amino acid sequence with at least about 90%, 91%, 92%, 93%, 94%, 95%, 96%, 97%, 98% or 99% sequence identity to SEQ ID NO: 1 and wherein the amino acid sequence has one set of amino acid substitutions set forth in Table 5.

In some embodiments, a botulinum neurotoxin protein or fragment thereof comprising an amino acid sequence relative to the L-chain protease of botulinum neurotoxin serotype A (SEQ ID NO: 1) is provided, where the modified amino acid sequence comprises one of the sets of amino acid substitutions set forth in Table 6. A botulinum neurotoxin protein is provided that comprises an amino acid sequence of with at least about 90%, 91%, 92%, 93%, 94%, 95%, 96%, 97%, 98% or 99% sequence identity to SEQ ID NO: 1 and wherein the amino acid sequence has one set of amino acid substitutions set forth in Table 6.

In some embodiments, a botulinum neurotoxin protein or fragment thereof comprises an amino acid sequence that has at least one of the following amino acid substitutions relative to the L-chain protease of botulinum neurotoxin serotype A (SEQ ID NO: 1):
(i) N53H;
(ii) E148Y:
(iii) K166F:
(iv) E148Y and K166F;
(v) S254L; or
(vi) S254M.
   A botulinum neurotoxin protein is provided that comprises an amino acid sequence with at least about 90%, 91%, 92%, 93%, 94%, 95%, 96%, 97%, 98% or 99% sequence identity to SEQ ID NO: 1 and wherein the amino acid sequence has one of the following amino acid substitutions: (i)N53H; (ii) E148Y; (iii) K166F; (iv) E148Y and K166F; (v) S254L; or (vi) S254M.

In some embodiments, a botulinum neurotoxin protein or fragment thereof, is provided that comprises an amino acid sequence having at least about 90%, e.g., about 90%, about 91%, about 92%, about 93%, about 94%, about 95%, about 96%, about 97%, about 98%, or about 99%, sequence identity to SEQ ID NO: 1 and two or more amino acid substitutions selected from the group consisting of: N26X1, wherein X1 is S, T, M, or C; A27X2, wherein X2 is L, R, I, V, M, K, or Q; Q29X3, wherein X3 is R, S, K, M, I, or T; N53X4, wherein X4 is H, R, Q, K, M, or I; E55X5, wherein X5 is I, N, V, L, M, Q, H, or D; E56X6, wherein X6 is I, L, V, or M; Q162X7, wherein X7 is R, K, M, or I; E201X8, wherein X8 is D, N, or Q; D203X9, wherein X9 is V, I, L, or M; N240X10, wherein X10 is A, S, G, T, M, or C; 5254X11, wherein X11 is A, L, M, I, V, G, or C; K364X12, wherein X12 is R, Q, M, or I; and Y387X13, wherein X13 is N, Q, H, E, or D. In some embodiments, the botulinum neurotoxin protein or fragment further comprises one or more additional amino acid substitutions selected from the group consisting of: E148X14, wherein X14 is Y, W, F, or H; K166X15, wherein X15 is F, M, L, Y, W, or H; and G305X16, wherein X16 is G, D, E, N, or Q. In some embodiments, X1 of N26X1 is S. In some embodiments, X2 of A27X2 is L or R. In some embodiments, X3 of Q29X3 is R or S. In some embodiments, X4 of N53X4 is H or R. In some embodiments, X5 of E55X5 is I, N, or V. In some embodiments, X6 of E56X6 is I. In some embodiments, X7 of Q162X7 is R. In some embodiments, X8 of E201X8 is D. In some embodiments, X9 of D203X9 is V. In some embodiments, X10 of N240X10 is A or S. In some embodiments, X11 of 5254X11 is A, L, or M. In some embodiments, X12 of K364X12 is R. In some embodiments, X13 of Y387X13 is N. In some embodiments, X14 of E148X14 is Y. In some embodiments, X15 of K166X15 is F. In some embodiments, X16 of G305X16 is G or D.

In some embodiments, a botulinum neurotoxin protein or fragment thereof, is provided that comprises an amino acid sequence having at least about 90%, e.g., about 90%, about 91%, about 92%, about 93%, about 94%, about 95%, about 96%, about 97%, about 98%, or about 99%, sequence identity to SEQ ID NO: 1 and (a) at least amino acid substitution selected from the group consisting of: N26X1, wherein X1 is S, T, M, or C; A27X2, wherein X2 is L, R, I, V, M, K, or Q; Q29X3, wherein X3 is R, S, K, M, I, or T; N53X4, wherein X4 is H, R, Q, K, M, or I; E55X5, wherein X5 is I, N, V, L, M, Q, H, or D; E56X6, wherein X6 is I, L, V, or M; Q162X7, wherein X7 is R, K, M, or I; E201X8, wherein X8 is D, N, or Q; D203X9, wherein X9 is V, I, L, or M; N240X10, wherein X10 is A, S, G, C, T, or M; S254X11, wherein X11 is A, L, M, I, V, G, or C; K364X12, wherein X12 is R, Q, M, or I; and Y387X13, wherein X13 is N, Q, H, E, or D, and (b) at least one amino acid substitution selected from the group consisting of: E148X14, wherein X14 is Y, W, F, or H; K166X15, wherein X15 is F, M, L, Y, W, or H; and G305X16, wherein X16 is G, D, E, N, or Q, and with the proviso that the amino acid sequence is not SEQ ID NO: 27. In some embodiments, X1 of N26X1 is S. In some embodiments, X2 of A27X2 is L or R. In some embodiments, X3 of Q29X3 is R or S. In some embodiments, X4 of N53X4 is H or R. In some embodiments, X5 of E55X5 is I, N, or V. In some embodiments, X6 of E56X6 is I. In some embodiments, X7 of Q162X7 is R. In some embodiments, X8 of E201X8 is D. In some embodiments, X9 of D203X9 is V. In some embodiments, X10 of N240X10 is A or S. In some embodiments, X11 of 5254X11 is A, L, or M. In some embodiments, X12 of K364X12 is R. In some embodiments, X13 of Y387X13 is N. In some embodiments, X14 of E148X14 is Y. In some embodiments, X15 of K166X15 is F. In some embodiments, X16 of G305X16 is G or D. In some embodiments, the botulinum neurotoxin protein or fragment thereof, comprises an amino acid sequence having at least about 90% sequence identity to SEQ ID NO: 1 and amino acid substitution 5254X11 wherein X11 is L or M and/or amino acid substitution N53H, and optionally further including one or more of the following amino acid substitutions: N26S, Q29R, E55V, E148Y, K166F, N240A, G305D. In some embodiments, the botulinum neurotoxin protein or fragment thereof, comprises an amino acid sequence having at least about 90% sequence identity to SEQ ID NO: 1 and amino acid substitutions E201D and D203V, and optionally further including one or more of the following amino acid substitutions: K166F, N240A, S254A, G305D.

In some embodiments, the botulinum neurotoxin protein or fragment thereof comprises the amino acid substitution of S254M, with reference to SEQ ID NO: 1.

In some embodiments, the botulinum neurotoxin protein or fragment thereof comprises the amino acid substitution of S254L with reference to SEQ ID NO: 1.

In some embodiments, the botulinum neurotoxin protein or fragment thereof comprises the amino acid substitution of N53H with reference to SEQ ID NO: 1.

In some embodiments, the botulinum neurotoxin protein or fragment thereof cleaves human SNAP23. In some embodiments, the botulinum neurotoxin protein or fragment thereof is at least about 1.5, 2.0, 2.5, 3.0, 3.5, 4.0, 4.5, 5.0, 6.0, 7.0, 7.5, 8.0, 9.0, 10, 12, 15, 20 or 25 fold more specific for SNAP23 than for SNAP25.

In still some embodiments, the botulinum neurotoxin protein or fragment thereof is at least about 5, 10, 25, 50, 75, 100, 120, 125, 130, 150, 200, 300, 400, 500, 600, 700, 800, 900, 1000, 1100, 1200, 1300, 1400 or 1500 fold more specific for cleaving SNAP23 than a botulinum neurotoxin protein altered or modified only at these four positions relative to SEQ ID NO: 1 as follows: E148Y, K166F, S254A, G305D.

In yet some embodiments, the botulinum neurotoxin protein or fragment thereof is at least about 30, 40, 50, 60, 70, 80, 90, 100, 110, 120, 130, 140, 150, 175, 200, 225, 250, 275, 300, 500, 750 or 1200 fold more specific for cleaving SNAP23 than a botulinum neurotoxin protein modified only at these four positions relative to SEQ ID NO: 1 as follows: E148Y, K166F, S254A, G305D.

In some embodiments, the botulinum neurotoxin protein or fragment thereof is at least about 40 fold more specific for cleaving SNAP23 than a botulinum neurotoxin protein modified only at these four positions relative to SEQ ID NO: 1 as follows: E148Y, K166F, S254A, G305D.

In some embodiments, the botulinum neurotoxin protein or fragment thereof is at least about 100 fold more specific for cleaving SNAP23 than a botulinum neurotoxin protein with modifications only at these four positions relative to SEQ ID NO: 1 as follows: E148Y, K166F, S254A, G305D.

In some embodiments, the botulinum neurotoxin protein or fragment thereof is at least about 1300 fold more specific for cleaving SNAP23 than a botulinum neurotoxin protein modified only at these four positions relative to SEQ ID NO: 1 as follows: E148Y, K166F, S254A, G305D. In some embodiments, the at least about 1300 fold more SNAP23 cleavage specificity is obtained under physiological salt conditions. In some embodiments, the physiological salt conditions include 50 mM KH₂PO₄ at pH 7.4. In some embodiments, in physiologicial sal conditions supplemented with zinc (50 mM KH₂PO₄, 0.2 nM ZnCl₂ pH 7.4), the botulinum neurotoxin protein or fragment thereof is at least about 120 fold more specific for cleaving SNAP23 than a botulinum neurotoxin protein modified only at these four positions relative to SEQ ID NO: 1 as follows: E148Y, K166F, S254A, G305D.

In some embodiments, the botulinum neurotoxin protein or fragment thereof has at least about 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% sequence identity to a botulinum neurotoxin protein of any one of the botulinum neurotoxin proteins modified compared to SEQ ID NO: 1 as described herein.

In some embodiments, the botulinum neurotoxin protein or fragment thereof comprises (i) a protein with at least about 80%, 85%, 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% sequence identity to a modified botulinum neurotoxin protein or fragment thereof described herein; or (ii) a protein identical to a modified botulinum neurotoxin protein or fragment thereof described herein.

In some embodiments, the botulinum neurotoxin protein or fragment thereof comprises (i) a protein with at least about 80%, 85%, 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% sequence identity to a modified botulinum neurotoxin protein or fragment thereof described herein; or (ii) a protein identical to a modified botulinum neurotoxin protein or fragment thereof described herein; and a heavy chain protein from a botulinum neurotoxin or fragment thereof is provided.

In some embodiments, the heavy chain protein is from botulinum neurotoxin serotype A.

In some embodiments, a botulinum neurotoxin protein or fragment thereof comprises an amino acid sequence relative to the L-chain protease of botulinum neurotoxin serotype E of SEQ ID NO: 28 or of SEQ ID NO: 31 that is modified as described herein. In some embodiments, the botulinum neurotoxin protein or fragment thereof comprises an amino acid sequence comprising at least about 80%, 85%, 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% sequence identity to the botulinum neurotoxin protein of SEQ ID NO: 28 or SEQ ID NO: 31.

In some embodiments, the botulinum neurotoxin protein or fragment thereof comprises (i) a protein with at least about 80%, 85%, 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% sequence identity to a botulinum neurotoxin protein, or fragment thereof, of SEQ ID NO: 28 or SEQ ID NO: 31; or (ii) a protein identical to the botulinum neurotoxin protein of SEQ ID NO: 28 or SEQ ID NO: 31, or fragment thereof.

In some embodiments, the botulinum neurotoxin protein or fragment thereof comprises (i) a protein having at least about 80%, 85%, 90%, 91%, 92%, 93%, 94%, 95%, 95.5%, 96%, 96.5%, 97%, 97.5%, 98%, 98.5%, 99%, 99.1%, 99.2%, 99.3%, 99.4%, 99.5%, 99.6%, 99.7%, 99.8%, 99.9% sequence identity to the botulinum neurotoxin protein or fragment thereof of SEQ ID NO: 28 or SEQ ID NO: 31; or (ii) a protein identical to the botulinum neurotoxin protein of SEQ ID NO: 28 or SEQ ID NO: 31; and a heavy chain protein from a botulinum neurotoxin or fragment thereof. In some embodiments, the heavy chain protein is from botulinum neurotoxin serotype E.

The light-chain of a botulinum neurotoxin provides a protease function (also known as non-cytotoxic protease function), and commonly has a molecular weight of about 50 kDa. Such non-cytotoxic proteases may act by proteolytically cleaving intracellular transport proteins known as SNARE proteins (e.g., SNAP-25, VAMP, or Syntaxin) (see Gerald K (2002) “Cell and Molecular Biology” (4th edition) John Wiley & Sons, Inc.). The naturally occurring (wild-type) BoNT/A L-chain is more particularly capable of efficiently cleaving SNAP-25, but has only de minimis capability of cleaving hSNAP-23. The modified BoNT/A L-chain proteases herein differ from the naturally occurring BoNT/A L-chain in their ability to cleave hSNAP-23. The modified BoNT/E L-chain proteases herein differ from the naturally occurring BoNT/E L-chain in their ability to cleave hSNAP-29.

The botulinum neurotoxin proteins described herein are also referred to herein as “modified”, “variant”, “mutant”, “protein variant” or “protease variant”, and intend a clostridial neurotoxin with an amino acid sequence that has been modified by the replacement, substitution, alteration, addition or deletion of at least one amino acid, relative to a wild-type botulinum toxin serotype A, B, C, D, E, F or G which is recognized by a target cell, internalized by the target cell, and catalytically cleaves a SNARE (SNAP (Soluble NSF Attachment Protein) Receptor) protein in a target cell. An example of a variant or “modified” botulinum neurotoxin is a variant light chain of a botulinum toxin having one or more amino acids substituted, altered, deleted and/or added relative to the light chain of a wild-type botulinum neurotoxin, generally of the same serotype. This variant light chain may have the same or better ability to prevent exocytosis, for example, the release of neurotransmitter vesicles. Additionally, the biological effect of a variant may be decreased compared to the parent chemical entity. For example, a variant light chain of a botulinum toxin type A having an amino acid sequence removed may have a shorter biological persistence than that of the parent (or native) botulinum toxin type A light chain.

In some embodiments, the botulinum neurotoxin can be a modified neurotoxin, that is a botulinum neurotoxin which has at least one of its amino acids deleted, substituted, altered, modified or replaced, as compared to a native toxin, or the modified botulinum neurotoxin can be a recombinant produced botulinum neurotoxin or a derivative or fragment thereof. In some embodiments, the modified toxin has an altered cell targeting capability for a neuronal or non-neuronal cell of interest. This altered capability is achieved by replacing the naturally occurring targeting domain of a botulinum toxin with a targeting domain showing a specific binding activity for a non-botulinum toxin receptor present in a non-botulinum toxin target cell. Such modifications to a targeting domain result in a modified toxin that is able to specifically bind to a non-botulinum toxin receptor (target receptor) present on a non-botulinum toxin target cell (re-targeted). A modified botulinum toxin with a targeting activity for a non-botulinum toxin target cell can bind to a receptor present on the non-botulinum toxin target cell, translocate into the cytoplasm, and exert its proteolytic effect on the SNARE complex of the target cell. In essence, a botulinum toxin light chain comprising an enzymatic domain is intracellularly delivered to any desired cell by selecting the appropriate targeting domain.

In some embodiments, the clostridial derivative is a botulinum toxin, which is selected from the group consisting of botulinum toxin types A, B, C₁, D, E, F and G and mosaics (CD, DC, FA) and non-clostridial BoNT-like encoding sequences (BoNT/X, BoNT/Wo, BoNT/En (eBoNT/J), Cp1, PMP1) In some embodiments, the clostridial derivative of the present method is a botulinum toxin type A. The botulinum toxin can be a recombinant botulinum neurotoxin, such as botulinum toxins produced by E. coli.

In some embodiments, modified BoNT/A and BoNT/E light chains that are substantially homologous, e.g., are functional variants or homologs, and which exhibit improved substrate binding to and/or cleavage of non-canonical substrates, e.g., non-neuronal SNARE proteins such as human SNAP-23 (hSNAP-23) and/or SNAP-29 (hSNAP-29) are contemplated. These functional variants or homologs can be characterized as having one or more amino acid mutations (such as an amino acid deletion, addition, and/or substitution) other than the ones disclosed herein with regard to hSNAP-23 and/or hSNAP-29 cleavage, and which do not significantly affect the folding or protease activity, in particular hSNAP-23 cleavage. For example, such mutations include, without limitation, conservative substitutions, small deletions (e.g., of 1 to about 30 amino acids), small amino- or carboxyl-terminal extensions (such as an amino-terminal methionine residue), and addition of a small linker peptide of up to about 20-25 residues or of an affinity tag.

Functional variants or homologs may comprise mutations of minor nature, such as conservative amino acid substitutions. Conservative amino acid substitutions include, without limitation:
Basic: arginine, lysine, histidine
Acidic: glutamic acid, aspartic acid
Polar: glutamine, asparagine
Hydrophobic: leucine, isoleucine, valine, methionine
Aromatic: phenylalanine, tryptophan, tyrosine
Small: glycine, alanine, serine, threonine

In addition to the 20 standard amino acids, non-standard amino acids (such as 4-hydroxyproline, 6-/V-methyl lysine, 2-aminoisobutyric acid, isovaline and α-methyl serine) may be substituted for amino acid residues of the polypeptides. Non-conservative amino acids, amino acids that are not encoded by the genetic code, and unnatural amino acids may be substituted for clostridial polypeptide amino acid residues. The polypeptides may also comprise non-naturally occurring amino acid residues.

Non-naturally occurring amino acids include, without limitation, trans-3-methylproline, 2,4-methano-proline, cis-4-hydroxyproline, trans-4-hydroxy-proline, N-methylglycine, allo-threonine, methyl-threonine, hydroxy-ethylcysteine, hydroxyethylhomo-cysteine, nitro-glutamine, homoglutamine, pipecolic acid, tert-leucine, norvaline, 2-azaphenylalanine, 3-azaphenyl-alanine, 4-azaphenyl-alanine, and 4-fluorophenylalanine. Methods in the art may be used to incorporate non-naturally occurring amino acid residues into proteins.

The amino acid substitution may comprise the substitution of an amino acid comprising a physiochemical property (e.g., hydrophobicity) with an amino acid having a similar or alternative property. Examples of such substitutions are listed below:
Acidic amino acid substituted for a neutral, polar amino acid;
Polar amino acid substituted for a non-polar amino acid;
Non-polar amino acid substituted for a non-polar amino acid;
Non-polar amino acid substituted for a polar amino acid;
Polar amino acid substituted for a basic amino acid;
Non-polar amino acid substituted for an acidic amino acid;
Non-polar amino acid substituted for a polar amino acid.

Accordingly, the L-chain of all BoNT/A subtypes, such as any of BoNT/A1 to BoNT/A8 L-chain, which comprise one or more of the mutations as described herein for cleavage of hSNAP-23, are contemplated. Said BoNT/A L-chain may additionally comprise further mutations to provide a non-native activation cleavage site, such as the cleavage site of enterokinase (SEQ ID NO: 10), PreScission, Factor Xa, Thrombin, TEV protease, or a non-native activation cleavage site located between the cysteine (C) residues that form the interchain disulfide bridge between the light chain and heavy chain (see, for example, C1 and C25 of SEQ ID NO: 32).

Methods for engineering and/or modifying a protease domain of a botulinum neurotoxin (BoNT) L-chain (LC) protease (designated herein as LC/A protease or LC protease) to bind and/or cleave a non-canonical substrate are also provided. In some embodiments, the method comprises (i) identifying sites in a protease domain of a botulinum neurotoxin involved in substrate binding and/or catalysis; (ii) constructing a library of protease domain gene mutants of botulinum neurotoxin for the identified sites; (iii) transforming each gene mutant in the library into an expression system; (iv) expressing protein from clonal populations of each expression system; (v) testing the expressed protein for binding to or cleavage of a non-canonical substrate to identify expressed proteins with improved substrate binding and/or cleavage; (vi) sequencing protein identified to have improved substrate binding and/or cleavage; and (vii) repeating steps (ii)-(vi) using the sequence identified in (vi), optionally including repeating step (i).

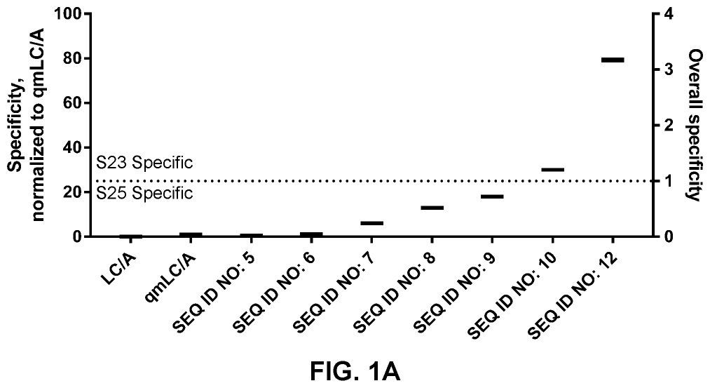

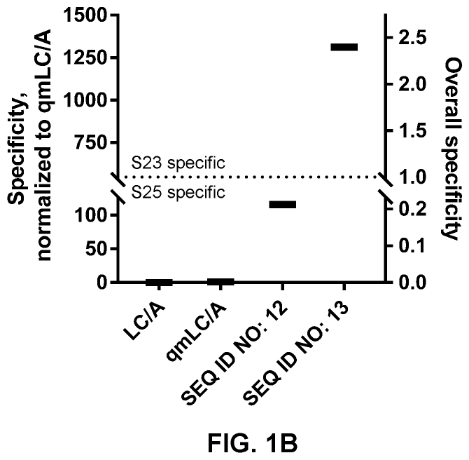

In some embodiments, structure-guided analysis and random/shuffling mutagenesis techniques (e.g., error-prone PCR and staggered extension process) were used to identify sites (also known as “hot spots”) in LC/A proteases that contribute to substrate binding and catalysis. In some embodiments, DNA libraries of LC/A protease gene variants that probe these sites or hot spots were constructed with several small-diversity libraries with 9 to 12 amino acids substituted at single sites following an iterative saturation mutagenesis approach (Reetz, M. T. & Carballeira, J. D. Nat. Protoc. 2, 891-903 (2007)). FIGS. 1A and 1B demonstrate an improved specificity of exemplary LC/A proteases for SNAP23 over SNAP25.

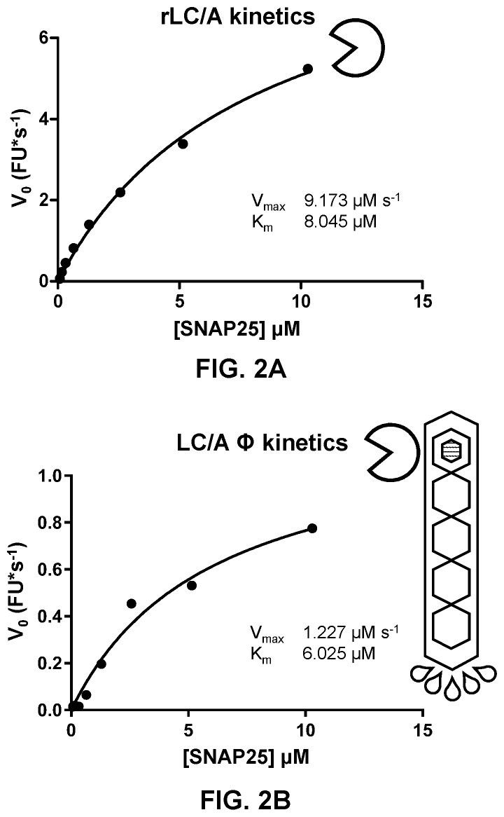

To confirm that an exemplary active wild-type (WT) LC/A protease (SEQ ID NO: 1) was successfully displayed on the P8 protein of M13 bacteriophage (LC/A Φ), affinity of LC/A Φ was compared to a recombinantly expressed and purified WT LC/A (rLC/A). Depletion of substrate was detected by a fluorescence resonance energy transfer (FRET) assay and the results are provided in FIGS. 2A and 2B. The data demonstrates that the exemplary active WT LC/A protease was successfully displayed on LC/A Φ as determined by comparing the affinity of rLC/A (shown in FIG. 2A) to the activity of LC/A Φ (shown in FIG. 2B). This assay can be adapted and employed on any BoNT protease.

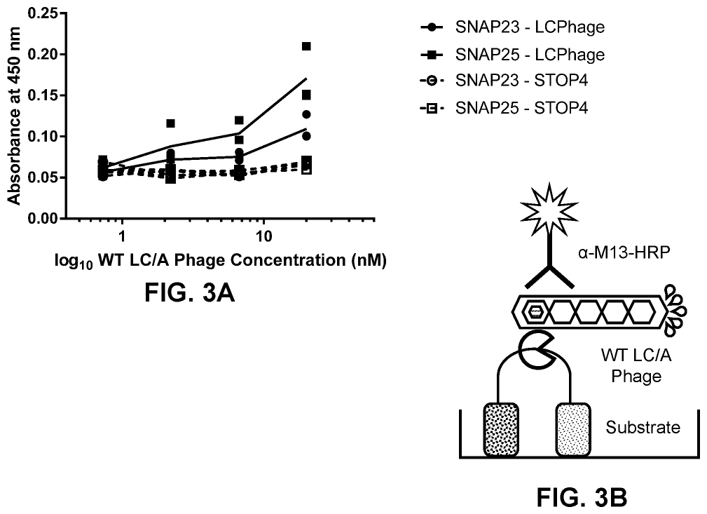

In another study, ELISA was used to detect SNAP23 and SNAP25 substrate binding by WT LC/A phage. FIG. 3A demonstrates SNAP23 and SNAP25 substrate binding by WT LC/A phage as determined by ELISA. A schematic illustration of the exemplary complex formed by the binding of an anti-M13-HRP antibody, WT LC/A phage, and substrate is shown in FIG. 3B. STOP4 phage with no displayed protein was used as a negative control for the assay. This assay can be employed on any BoNT protease.

Methods of growing cells for expressing LC/A proteases, including LC/A protease gene variants of the DNA libraries described herein, are also provided. LC/A protease genes and gene variant DNA libraries were subcloned into an expression vector and transformed into E. coli cells for growth on LB/agar plates. Colonies were picked for inoculating 96 deep-well plate (DWP) for growth and protein expression. This exemplary method utilizes E. coli expression system, however other expression systems are contemplated, including but not limited to yeast (for example Pichia), baculovirus in insect cell, cell-free expression, mammalian cell lines, animals, and phage.

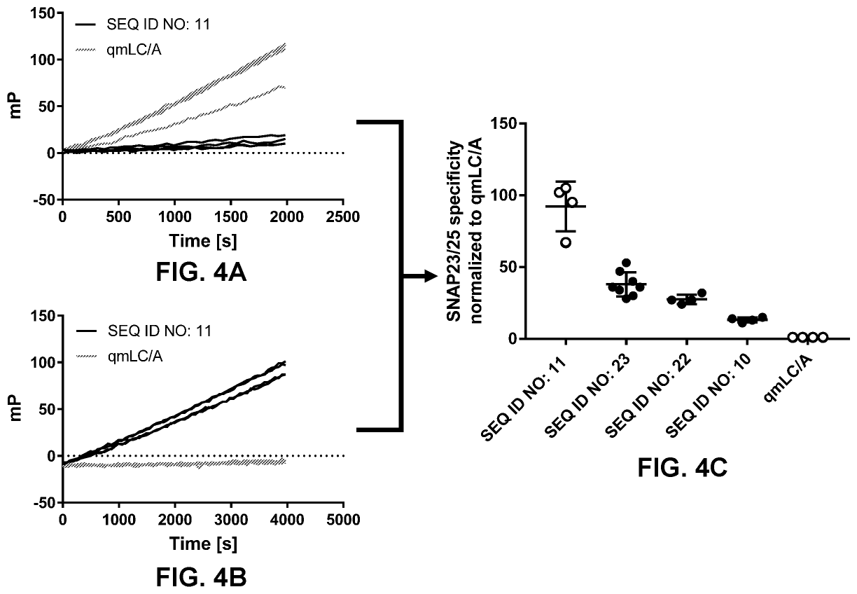

Substrate specificity of the exemplary modified LC/A proteases was evaluated, and the data is shown in FIGS. 4A-4C. The slopes of initial cleavage rates of SNAP25 (SEQ ID NO: 25) (FIG. 4A) and SNAP23 (SEQ ID NO: 24) (FIG. 4B) by the modified LC/A proteases were divided and normalized to the corresponding cleavage rates of the protease variant of SEQ ID NO: 27 (used as the reference protease) to determine improvements in SNAP23 specificity (FIG. 4C). The data demonstrates that the exemplary modified LC/A proteases represented in FIG. 4C, specifically the modified LC/A protease of SEQ ID NO: 11 (which includes a S254L substitution) was over 100-fold more specific and the modified LC/A protease of SEQ ID NO: 23 (which includes a S254M substitution) was over 40-fold more specific for SNAP23 over SNAP25 than the protease variant of SEQ ID NO: 27 (used as the reference protease). This assay can be adapted and employed on any BoNT protease.

Table 7 details SNAP23 and SNAP25 cleavage specificity for the modified LC/A proteases. The modified proteases demonstrate both a higher rate of SNAP23 cleavage and substrate inhibition with SNAP25. As shown in Table 7, an exemplary modified LC/A protease with the following amino acid substitutions relative to the wild type protease (SEQ ID NO: 1): N26S, Q29R, N53R, E55V, E148Y, K166F, N240A, and S254L (SEQ ID NO: 12) demonstrates an increase in specificity for SNAP23 of about 100-fold or more over the reference protease variant of SEQ ID NO: 27 in assay conditions for LC/A (assay buffer: 50 mM HEPES, 0.05% Tween, pH 7.4). The addition of S254L mutation was shown to increase SNAP23 specificity of the modified protease by about 100-fold or more over the reference protease. As also shown in Table 7, another exemplary modified LC/A protease with N26S, Q29R, N53H, E55V, E148Y, K166F, N240A, and S254L substitutions (SEQ ID NO: 13) demonstrated an increase in specificity for SNAP23 of about 1300-fold or more over the reference protease variant of SEQ ID NO: 27 in buffer that approximates intracellular conditions (Intracellular Buffer, 50 mM KH₂PO₄ pH 7.4). The addition of N53H substitution was shown to increase SNAP23 specificity of the protease by about 1300-fold or more over the reference protease variant of SEQ ID NO: 27 in intracellular buffer. As shown in Table 8, the protease variant of SEQ ID NO: 13 demonstrated an increase in specificity for SNAP23 of about 120-fold or more over the protease variant of SEQ ID NO: 27 in intracellular salt conditions supplemented with zinc (50 mM KH₂PO₄, 0.2 nM ZnCl₂ pH 7.4).

Further, kinetic studies determined that D305 in the reference protease can be reverted to the wild-type residue (G) without affecting specificity and E148 and K166F mutations were confirmed to be mutations for SNAP23 specificity. Such a higher rate of substrate inhibition with the neuronal SNAP25 represents an improvement in safety for the evolved LC/A protease variants capable of limiting off-target cleavage events. To summarize, the LC/A protease variants described herein account for both decrease in native substrate activity and increase in target substrate activity resulting in an evolution of overall substrate specificity.

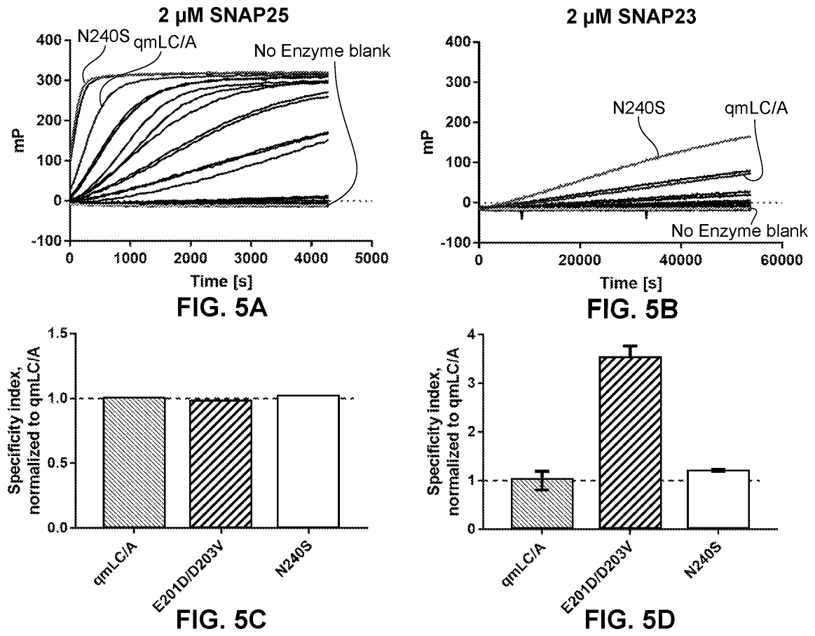

In another study, LC/A protease gene variants were generated with error-prone PCR (epPCR) technique for developing modified LC/A proteases with improved SNAP23 specificity. Using the method, botulinum neurotoxin proteins, also referred to herein as modified botulinum neurotoxin proteins, were identified, prepared, and tested for improved cleavage of SNAP23. Results are shown in FIGS. 5A-5D.

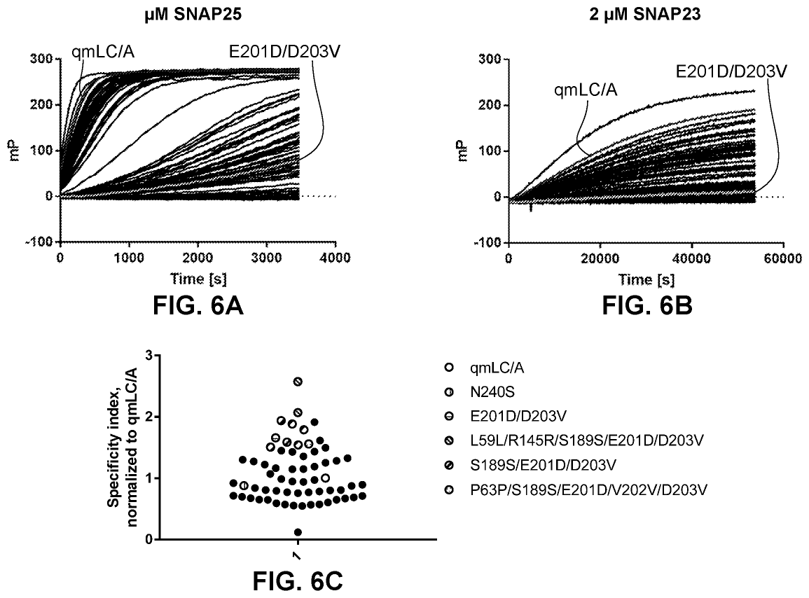

In another study, LC/A protease gene variants were generated with DNA shuffling technique for developing modified LC/A proteases with improved SNAP23 specificity. The LC/A protease gene variant library was grown and screened, and the proteins were tested for specificity. FIGS. 6A-6C show the specificity data of the LC/A proteases generated with DNA shuffling technique.

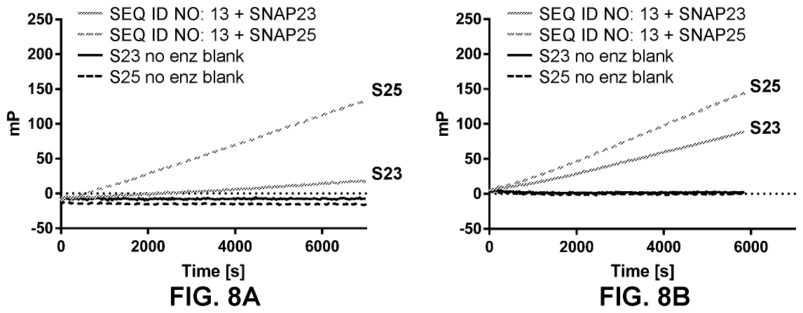

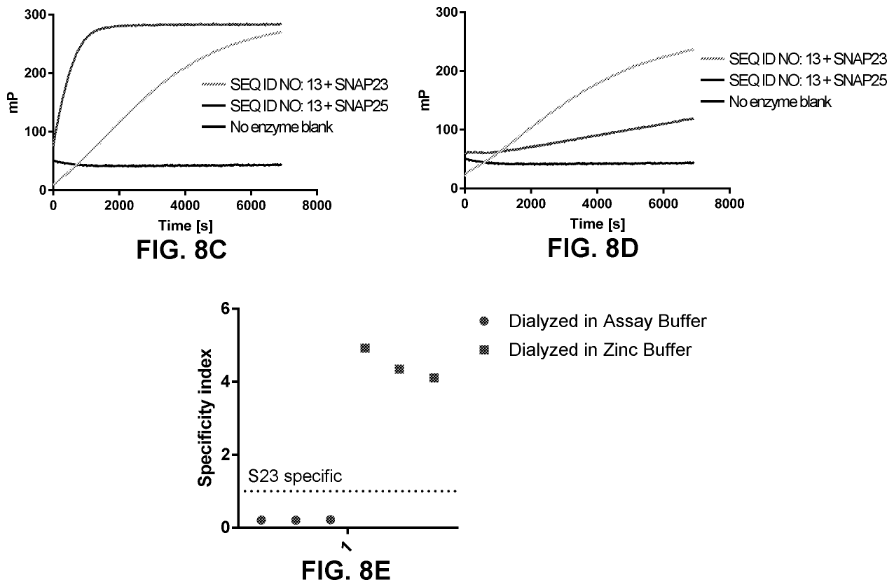

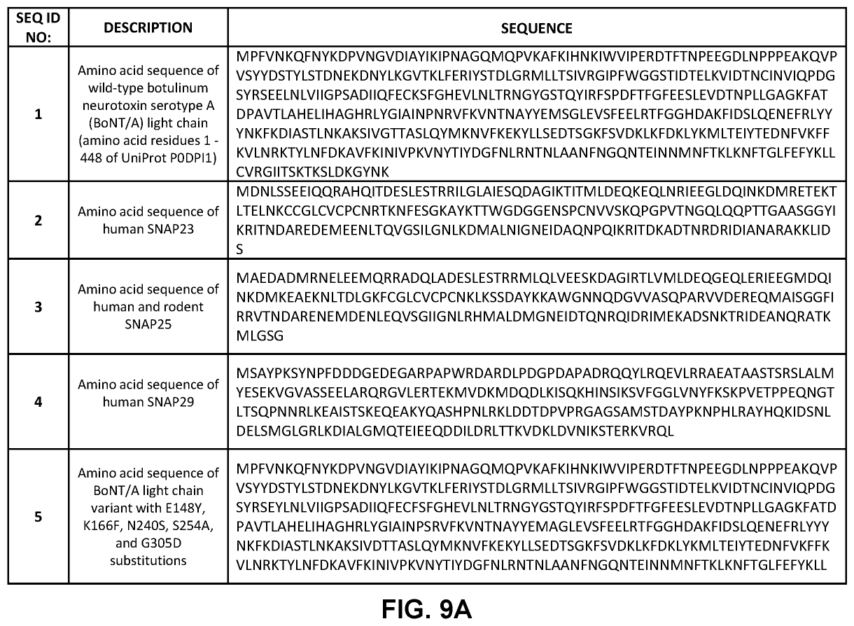

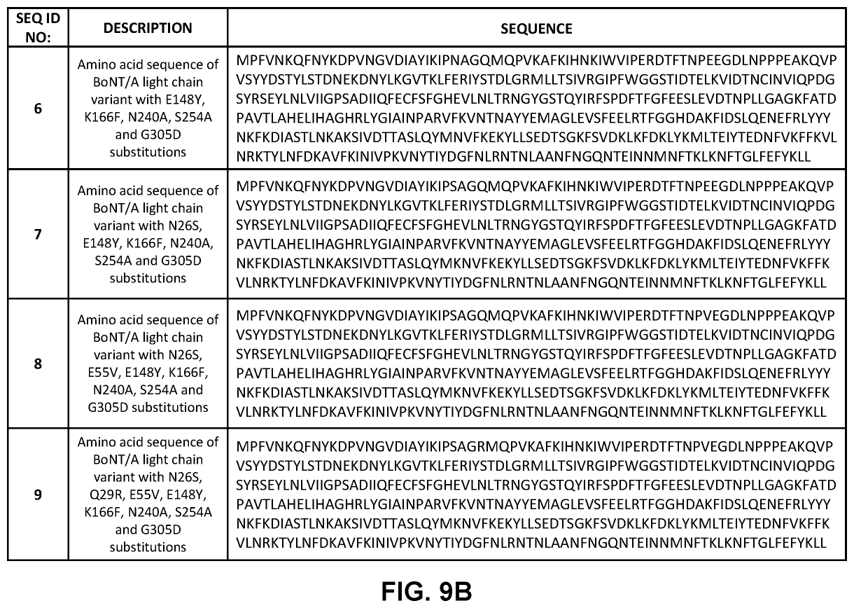

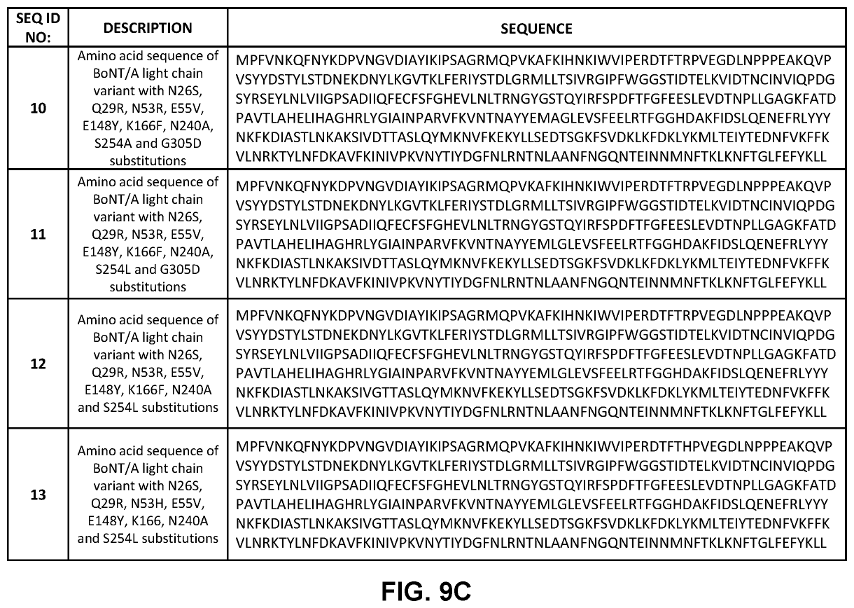

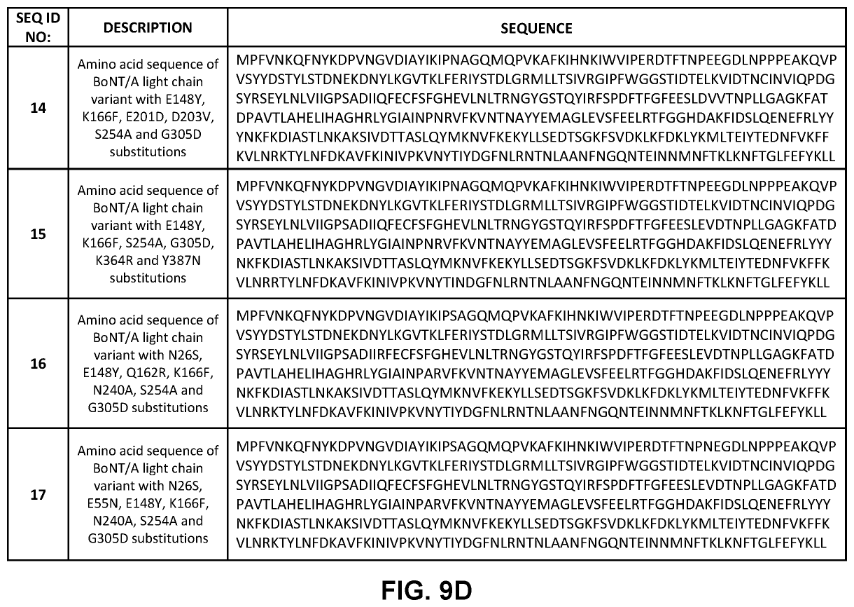

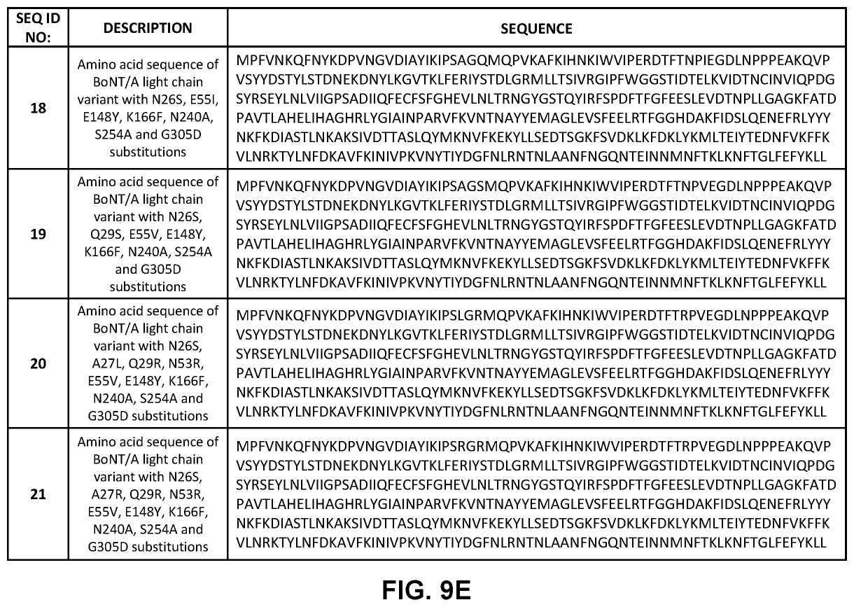

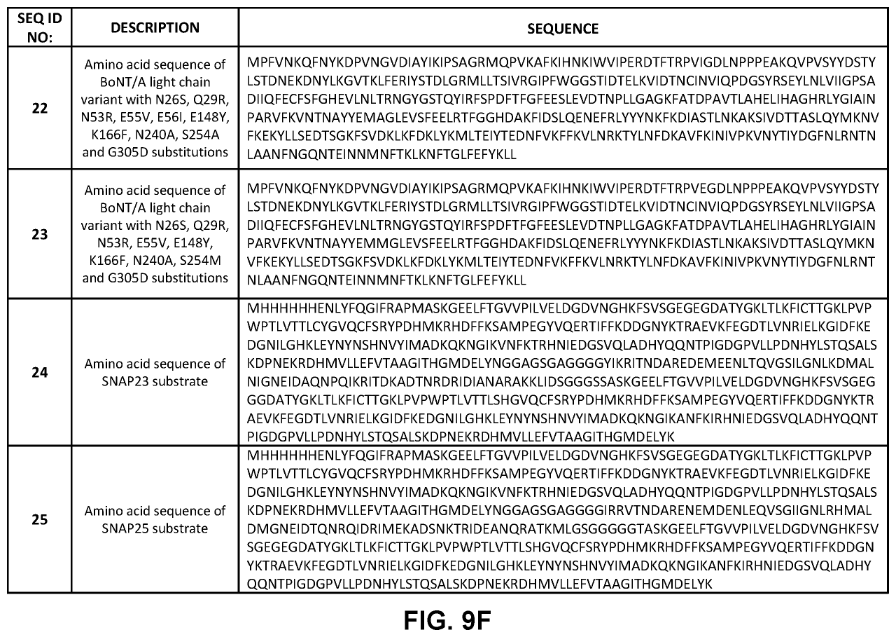

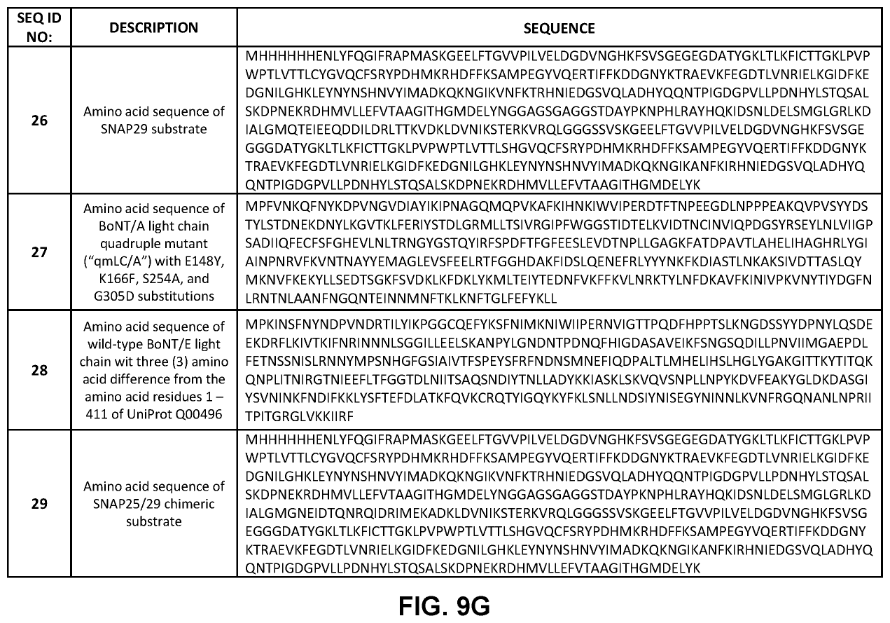

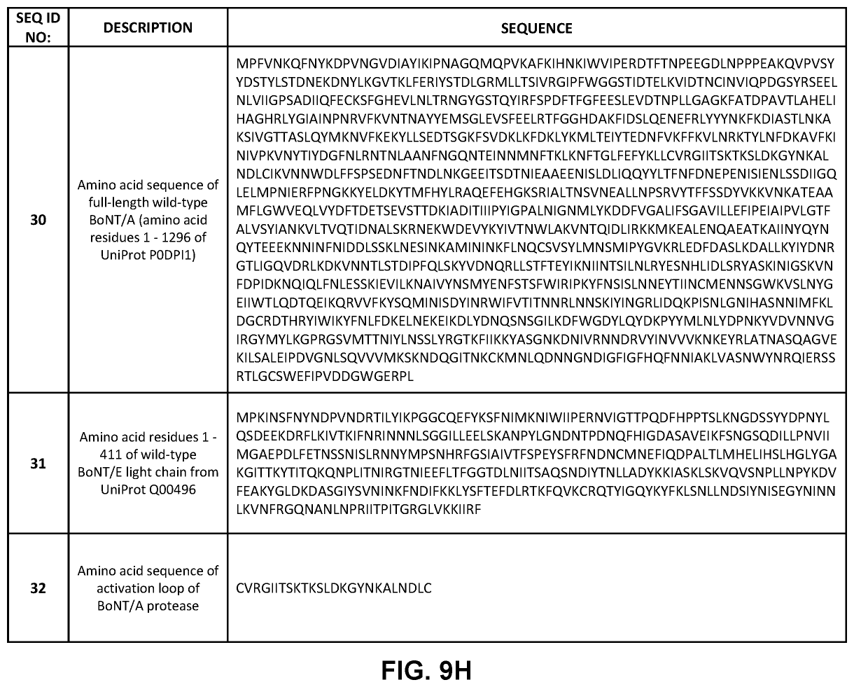

In another study, a novel method for modulating the substrate specificity of the LC/A proteases is described herein. FIGS. 8A-8E provide exemplary data demonstrating that the LC/A protease (SEQ ID NO: 13) dialyzed in zinc buffer cleaved a SNAP23 substrate with a higher rate than a SNAP25 substrate. The exemplary data demonstrates that the LC/A protease (SEQ ID NO: 13) exhibits strong dependence on the presence of Zn²⁺ for its substrate specificity, but not its activity. In the absence of additional Zn²⁺ (e.g., in assay buffer (50 mM HEPES, pH 7.4) or intracellular buffer (50 mM KH₂PO₄, pH 7.4)), the LC/A protease (SEQ ID NO: 13) remains proteolytic, but has a higher rate of cleavage for SNAP25 than SNAP23. The addition of 0.2 mM Zn²⁺ to either assay or intracellular buffer reverses this specificity, and the LC/A protease's specificity for SNAP23 is restored. Thus, the zinc-mediated modification provides a novel control element and/or a co-factor for modulating substrate specificities of the BoNT proteases.

It is understood that other cations (e.g., bivalent metal ions), small molecules, etc. can be used in place of Zn²⁺ to complete and/or facilitate formation of the BoNT protease-SNAP (e.g., LC/A-SNAP23 or LC/E-SNAP29) complex necessary for the improved protease activity according to the novel ligand-mediated method of modulating substrate specificity. In some embodiments, other suitable bivalent metal ions can also be used to offer a new level of enzymatic control for the proteases. Further, one could supplement a formulation with sufficient zinc to insure substrate specificity for therapeutic use.

Further, modifying the substrate specificity of the proteases with zinc revealed a new series of residues in the modified proteases that exert control over the protease substrate specificity. In particular, altering substrate specificity of the modified proteases was found to involve modification of seven residues occupying two loops (loop one spanning residues 26-29 and loop two spanning 52-56 of the LC/A protease of SEQ ID NO: 13), which are referred to herein as “substrate control” loops. Furthermore, the newly introduced Zn²⁺ binding site illustrates a novel method of ligand-based additional control over the substrate specificity function of the proteases. It is understood that such method for modulating the substrate specificity can be employed on any BoNT protease.

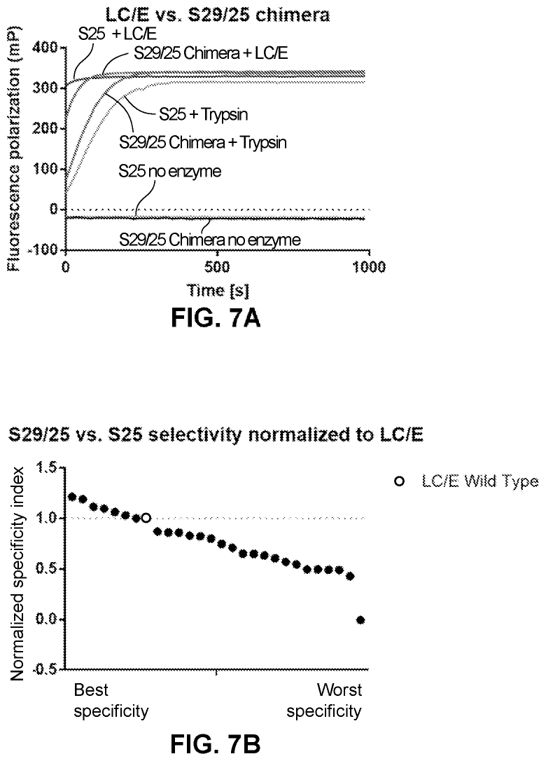

In another study, protease variants were screened against a native, target chimeric protein, then over rounds of evolution native amino acids (e.g., SNAP25) are swapped for target ones (e.g., SNAP29) until the substrate is 100% target. Using this technique, a SNAP25-29 chimeric substrate was created that is susceptible to LC/E cleavage. The chimeric substrate was screened against LC/E variants and the data is shown in FIGS. 7A-7B. The data demonstrates evolution of LC/E to cleave SNAP29 on the LC/A protease library platform via coevolution. In FIG. 7A, the SNAP29/25 chimeric substrate (19% SNAP25, 81% SNAP29) was cleaved by wild-type LC/E in a fluorescence-polarization assay. Trypsin, positive control. In FIG. 7B, LC/E variants screened against both the chimeric S29/25 substrate and SNAP25 revealed at least five modified LC/E proteases with potentially improved specificity for the chimera.

As can be appreciated, the methods described herein reveal the identification of amino acid positions within a wild-type BoNT/A L-chain to render a BoNT/A L-chain capable of hSNAP-23 cleavage. In this regard, introduction of an amino acid change (e.g., a mutation), may be affected by means of an amino acid a deletion, addition, insertion, or a substitution. Methods in the art may be used to allow introduction of such mutations. For example, it is possible to introduce a mutation by random or directed mutagenesis, by PCR using degenerate primers, e.g., in the nucleotide sequence coding for the protein of reference. Said techniques are notably described by Sambrook et al. in “Molecular Cloning: A laboratory Manual”, 4th edition, Cold Spring Harbor Laboratory Press, (2012, and updates from 2014), and by Ausubel et al. in “Current Protocols in Molecular Biology”, John Wiley & Sons (2012). The amino acid change occurs, in some embodiments, within one or more of the L-chain “binding pockets” relative to the wild-type BoNT/A L-chain (SEQ ID NO: 1).

In some embodiments, the botulinum neurotoxin protein or fragment thereof described herein has an improved specificity for a non-canonical substrate relative to its canonical substrate. In some embodiments, the canonical substrate is SNAP25 and the non-canonical substrate is SNAP23, SNAP29, or a SNAP25/29 chimeric substrate. In some embodiments, the canonical SNAP25 substrate comprises the amino acid sequence of SEQ ID NO: 25. In some embodiments, the non-canonical SNAP23 substrate comprises the amino acid sequence of SEQ ID NO: 24. In some embodiments, the non-canonical SNAP29 substrate comprises the amino acid sequence of SEQ ID NO: 4. In some embodiments, the non-canonical SNAP25/29 chimeric substrate comprises the amino acid sequence of SEQ ID NO: 29.

In some embodiments, the botulinum neurotoxin protein or fragment thereof is botulinum serotype A, B, C, D, E, F, G, a mosaic neurotoxin, a non-clostridial botulinum toxin-like encoding sequence, or combinations thereof.

In some embodiments, a nucleic acid encoding the botulinum neurotoxins, or fragment thereof, of the disclosure is provided.

In some embodiments, a plasmid is provided that comprises the nucleic acids described herein.

In some embodiments, a vector is provided that comprises the plasmids described herein.

In some embodiments, a host cell is provided that comprises the vectors described herein.

In some embodiments, an expression system is provided that comprises the host cells described herein.

In some embodiments, the expression system is selected from the group consisting of bacteria, yeast (for example Pichia), baculovirus in insect cell, cell-free expression, mammalian cell lines, animals, and phage. In some embodiments, the expression system is an E. coli expression system.

In some embodiments, a method of generating any one of the botulinum neurotoxin proteins, or fragment thereof, is provided, the method comprises culturing the host cell described herein under conditions sufficient for the expression of the botulinum neurotoxin protein or fragment thereof, and obtaining the botulinum neurotoxin protein or fragment thereof from the culture.

### Processing of and Alternate Sequences of Engineered Mutant Botulinum Neurotoxin Proteases

In some embodiments, various post-translational processing modifications and resulting alternate sequences of the botulinum neurotoxin proteins and fragments thereof are provided herein. In various embodiments, such post-translational processing may involve removal of the initiating methionine (M) amino acid, formation of disulfide bridges, limited proteolysis (nicking) and activation etc., depending on the expression system and expression conditions used for expression and purification of the botulinum neurotoxin proteins and fragments thereof. For example, in a bacterial expression system, post-translational processing of a gene product according to various embodiments may involve removal of the initiating methionine amino acid, formation of disulfide bridges, and/or limited proteolysis (nicking) by bacterial protease(s).

Accordingly, in some embodiments, the exemplary wild-type BoNT/A L-chain (SEQ ID NO: 1) and the botulinum neurotoxin protein sequences and fragments thereof (e.g., BoNT/A and BoNT/E proteases) disclosed herein (see, for example, SEQ ID NOS: 5-23, 27, 28, and 31) include the initiator methionine (M) amino acid as included in the translated gene product. It is understood that the initiator methionine may or may not be removed post-translationally depending on the expression system and expression conditions used for expression and purification of such proteins. For example, in a bacterial expression system, post-translational processing of a gene product according to various embodiments may involve removal of the initiating methionine. Accordingly, in some embodiments, the amino acid sequences of the botulinum neurotoxin protein and fragments thereof of SEQ ID NOS: 1, 5-23, 27, 28, and/or 31 are also contemplated without the initiator methionine (M).

In some embodiments, the exemplary BoNT/A L-chain amino acid sequence illustrated herein as SEQ ID NO: 1 is 448 amino acid residues in length and ends with K448, where the numbering includes the initiator methionine (M) as translated. It is understood that K438 of SEQ ID NO: 1 is the first lysine amino acid residue of the activation loop, whereas K448 of SEQ ID NO: 1 is the last lysine amino acid residue of the activation loop. In some embodiments, the activation loop of a BoNT/A protease comprises the exemplary amino acid sequence of SEQ ID NO: 32 and the activation loop is formed by the two cysteine residues (C1 and C25 of SEQ ID NO: 32) that forms a disulfide bond. In some embodiments, the BoNT/A protease is cleaved at both lysine (K) residues, e.g., at K338 and K448 of SEQ ID NO: 1, which removes the ten amino acid residues after K338. In such some embodiments, K338 most likely represents the C-terminal end of the L-chain after proteolytic cleavage of the activation loop. Thus, in some embodiments, the sequence encompassing amino acids 1-438 of SEQ ID NO: 1 represents the activated form of a wild-type BoNT/A L-chain (including the initiator methionine). In some embodiments, the sequence encompassing amino acids 1-438 of SEQ ID NO: 1 may represent the most naturally activated form of a wild-type BoNT/A L-chain with the initiator methionine included.

In this regard, it should be understood that prior to proteolytic activation, a wild-type BoNT/A L-chain is about 448 amino acid residues in length, which includes a short C-terminal extension of activation loop amino acid residues beyond the L-chain cysteine (C) that forms the interchain disulfide bridge to the H-chain. In some instances, the sequence encompassing amino acids 1-438 sequence of the BoNT/A1 L-chain may be isolated from the native protein. In some embodiments, BoNT/A L-chains of alternate lengths may be isolated following other native, incomplete or alternate proteolytic activation after other lysine residues in the activation loop. For example, in some embodiments, a native, incomplete or alternate proteolytic processing following K440 would yield a BoNT/A L-chain sequence of 1-440 amino acids. In some embodiments, a native, incomplete, or alternate proteolytic processing following K444 would yield a BoNT/A L-chain sequence of 1-444 amino acids. In yet some embodiments, a native, incomplete or alternate proteolytic processing following K448 would yield a BoNT/A L-chain sequence of 1-448 amino acids. It is understood that, while these are exemplary embodiments of native proteolytic processing of BoNT/A proteases (e.g., native complete as well as native incomplete or alternate proteolytic processing), engineered nicking of other amino acid residues at non-native activation cleavage site(s) in the activation loop of a botulinum neurotoxin protein, and fragments thereof, will provide alternate BoNT/A L-chain lengths as describe in detail below. It is understood that nicking at various native as well at non-native activation cleavage site(s) are also contemplated for BoNT/E proteases.

Accordingly, it is understood that, various native (e.g., natural, incomplete, and alternate) as well as engineered, non-native activation cleavage sites for proteolytic cleavage (nicking) in the amino acid sequences of the BoNT/A L-chain botulinum neurotoxin protein and fragments thereof of SEQ ID NOS: 1, 5-23 and/or 27 and BoNT/E L-chain botulinum neurotoxin protein and fragments thereof of SEQ ID NOS: 28 and 31, beyond those disclosed herein are also contemplated. It is also understood that native nicking (e.g., natural, incomplete, and alternate) at various, native activation cleavage site(s) and engineered nicking at non-native activation cleavage site(s) in the activation loop of the botulinum neurotoxin protein and fragments thereof will provide alternate BoNT/A and BoNT/E L-chain lengths beyond those disclosed herein. In some embodiments, alternate sequences of BoNT/A and BoNT/E activation loops are also contemplated for engineered nicking at non-native activation cleavage site(s).

Further, in some embodiments, it is contemplated that the amino acid sequence of any one of SEQ ID NO: 5-23, 27, 28 or 31 includes one or more of additional amino acid residues at the C-terminal. For example, in some embodiments, the amino acid sequence of any one of SEQ ID NO: 5-23 or 27 may include at its C-terminal end, part or all of the amino acid residue(s) of SEQ ID NO: 32. In some embodiments, the amino acid sequence of any one of SEQ ID NO: 5-23, 27, 28 or 31 may include at its C-terminal end, part or all of the amino acid residue(s) of an alternate activation loop, for example, for cleavage by alternate activation proteases.

### II. Methods of Modulating the Substrate Specificity of Botulinum Neurotoxin A

In another study, a method for modulating the substrate specificity of the botulinum neurotoxin proteins is described. In particular, the method comprises adding a cation to a composition comprising the protease. FIGS. 8A-8E provide exemplary data demonstrating that the modified LC/A protease (SEQ ID NO: 13) dialyzed in zinc buffer cleaved a SNAP23 substrate with a higher rate than a SNAP25 substrate. The modified LC/A protease (SEQ ID NO: 13) exhibits strong dependence on the presence of Zn²⁺ for its substrate specificity, but not its activity. In the absence of additional Zn²⁺ (e.g., in assay buffer (50 mM HEPES, pH 7.4) or intracellular buffer (50 mM KH₂PO₄, pH 7.4)), the modified LC/A protease (SEQ ID NO: 13) remains proteolytic, but has a higher rate of cleavage for SNAP25 than SNAP23. The addition of 0.2 mM Zn²⁺ to either assay or intracellular buffer reverses this specificity, and the modified LC/A protease's specificity for SNAP23 is restored. Thus, the zinc-mediated modification provides a novel control element and/or a co-factor for modulating substrate specificities of the BoNT proteases.

It is understood that according to various embodiments, other cations (e.g., bivalent metal ions), small molecules, etc. can be used in place of Zn²⁺ to complete and/or facilitate formation of the BoNT protease-SNAP (e.g., LC/A-SNAP23 or LC/E-SNAP29) complex necessary for improved protease activity. In some embodiments, other suitable bivalent metal ions can also be used to offer a new level of enzymatic control for the proteases. Further, one could supplement a formulation with sufficient zinc to insure substrate specificity for therapeutic use.

Further, modifying the substrate specificity of the proteases with zinc revealed a new series of residues in the modified proteases that exert control over the protease substrate specificity. In particular, altering substrate specificity of the modified proteases was found to involve modification of seven residues occupying two loops (loop one spanning residues 26-29 and loop two spanning 52-56 of the LC/A protease of SEQ ID NO: 13), which are referred to herein as “substrate control” loops. Furthermore, the newly introduced Zn²⁺ binding site illustrates a novel method of ligand-based additional control over the substrate specificity function of the proteases. It is understood that such method for modulating the substrate specificity can be employed on any BoNT protease.

### III. Examples

The following examples are illustrative in nature and are in no way intended to be limiting.

### Example 1

### Discussion of Exemplary Methods and Techniques

### A. Generation of Botulinum Neurotoxin Proteins

The botulinum neurotoxin proteins, also referred to as modified botulinum neurotoxin proteins, described herein may be derived from a primary sequence of a native peptide, or may be engineered using methods in the art. Such engineered peptides can be designed and/or selected because of enhanced or novel properties as compared with the native peptide. For example, peptides may be engineered to have increased enzyme reaction rates, increased or decreased binding affinity to a substrate or ligand, increased or decreased binding affinity to a receptor, altered specificity for a substrate, ligand, receptor or other binding partner, increased or decreased stability in vitro and/or in vivo, or increased or decreased immunogenicity in an animal.

### B. Mutations

### 1. Rational Design Mutation

The methods herein to identify and generate botulinum neurotoxin proteins enhance a desired biological activity or function, diminish an undesirable property of the peptide, and/or to add novel activities or functions to the protein, relative to its wild-type sequence. “Rational peptide design” may be used to generate such modified proteins. Once the amino acid sequence and structure of the protein or peptide is known and a desired mutation planned, the mutations can be made most conveniently to the corresponding nucleic acid codon which encodes the amino acid residue that is desired to be mutated. One of skill in the art can easily determine how the nucleic acid sequence should be altered based on the universal genetic code, and knowledge of codon preferences in the expression system of choice. A mutation in a codon may be made to change the amino acid residue that will be polymerized into the peptide during translation. Alternatively, a codon may be mutated so that the corresponding encoded amino acid residue is the same, but the codon choice is better suited to the desired peptide expression system. For example, cys-residues may be replaced with other amino acids to remove disulfide bonds from the mature peptide, catalytic domains may be mutated to alter biological activity, and in general, isoforms of the peptide can be engineered. Such mutations can be point mutations, deletions, insertions and truncations, among others.

Techniques in the art may be used to mutate specific amino acids in a peptide. The technique of site-directed mutagenesis, discussed above, is well suited for the directed mutation of codons. The oligonucleotide-mediated mutagenesis method is also discussed in detail in Sambrook et al. (2001, Molecular Cloning: A Laboratory Manual, Cold Spring Harbor Laboratory, New York, starting at page 15.51). Systematic deletions, insertions and truncations can be made using linker insertion mutagenesis, digestion with nuclease Bal31, and linker-scanning mutagenesis, among other methods in the art (Sambrook et al., 2001, Molecular Cloning: A Laboratory Manual, Cold Spring Harbor Laboratory, New York).

Rational peptide design has been successfully used to increase the stability of enzymes with respect to thermo-inactivation and oxidation. For example, the stability of an enzyme was improved by removal of asparagine residues in alpha-amylase (Declerck et al., 2000, J. Mol. Biol. 301:1041-1057), the introduction of more rigid structural elements such as proline into alpha-amylase (Igarashi et al., 1999, Biosci. Biotechnol. Biochem. 63:1535-1540) and D-xylose isomerase (Zhu et al., 1999, Peptide Eng. 12:635-638). Further, the introduction of additional hydrophobic contacts stabilized 3-isopropylmalate dehydrogenase (Akanuma et al., 1999, Eur. J. Biochem. 260:499-504) and formate dehydrogenase obtained from Pseudomonas sp. (Rojkova et al., 1999, FEBS Lett. 445:183-188). The mechanisms behind the stabilizing effect of these mutations is generally applicable to many peptides. These and similar mutations are contemplated to be useful with respect to the peptides described herein.

### 2. Random Mutagenesis Techniques

Botulinum neurotoxin proteins may be generated using techniques that introduce random mutations in the coding sequence of the nucleic acid. The nucleic acid is then expressed in a desired expression system, and the resulting peptide is assessed for properties of interest. Techniques in the art may be used to introduce random mutations into DNA sequences, and include PCR mutagenesis, saturation mutagenesis, and degenerate oligonucleotide approaches. See Sambrook and Russell (2001, Molecular Cloning, A Laboratory Approach, Cold Spring Harbor Press, Cold Spring Harbor, N.Y.) and Ausubel et al. (2002, Current Protocols in Molecular Biology, John Wiley & Sons, NY).

In PCR mutagenesis, reduced Taq polymerase fidelity is used to introduce random mutations into a cloned fragment of DNA (Leung et al., 1989, Technique 1:11-15). This is a very powerful and relatively rapid method of introducing random mutations into a DNA sequence. The DNA region to be mutagenized is amplified using the polymerase chain reaction (PCR) under conditions that reduce the fidelity of DNA synthesis by Taq DNA polymerase, e.g., by using an altered dGTP/dATP ratio and by adding Mn.sup.2+ to the PCR reaction. The pool of amplified DNA fragments is inserted into appropriate cloning vectors to provide random mutant libraries.

Saturation mutagenesis allows for the rapid introduction of a large number of single base substitutions into cloned DNA fragments (Mayers et al., 1985, Science 229:242). This technique includes generation of mutations, e.g., by chemical treatment or irradiation of single-stranded DNA in vitro, and synthesis of a complementary DNA strand. The mutation frequency can be modulated by modulating the severity of the treatment, and essentially all possible base substitutions can be obtained. Because this procedure does not involve a genetic selection for mutant fragments, both neutral substitutions as well as those that alter function, are obtained. The distribution of point mutations is not biased toward conserved sequence elements.

A library of nucleic acid homologs can also be generated from a set of degenerate oligonucleotide sequences. Chemical synthesis of a degenerate oligonucleotide sequences can be carried out in an automatic DNA synthesizer, and the synthetic genes may then be ligated into an appropriate expression vector. Methods in the art may be used for the synthesis of degenerate oligonucleotides (see for example, Narang, S A (1983) Tetrahedron 39:3; Itakura et al. (1981) Recombinant DNA, Proc 3rd Cleveland Sympos. Macromolecules, ed. A G Walton, Amsterdam: Elsevier pp. 273-289; Itakura et al. (1984) Annu. Rev. Biochem. 53:323; Itakura et al. (1984) Science 198:1056; Ike et al. (1983) Nucleic Acid Res. 11:477. Such techniques have been employed in the directed evolution of other peptides (see, for example, Scott et al. (1990) Science 249:386-390; Roberts et al. (1992) PNAS 89:2429-2433; Devlin et al. (1990) Science 249: 404-406; Cwirla et al. (1990) PNAS 87: 6378-6382; as well as U.S. Pat. Nos. 5,223,409, 5,198,346, and 5,096,815).

a. Directed Evolution

Botulinum neurotoxin proteins may also be generated using “directed evolution” techniques. In contrast to site directed mutagenesis techniques where knowledge of the structure of the peptide is required, there now exist strategies to generate libraries of mutations from which to obtain peptides with improved properties without knowledge of the structural features of the peptide. These strategies are generally known as “directed evolution” technologies and are different from traditional random mutagenesis procedures in that they involve subjecting the nucleic acid sequence encoding the peptide of interest to recursive rounds of mutation, screening and amplification.

In some “directed evolution” techniques, the diversity in the nucleic acids obtained is generated by mutation methods that randomly create point mutations in the nucleic acid sequence. The point mutation techniques include, but are not limited to, “error-prone PCR™” (Caldwell and Joyce, 1994; PCR Methods Appl. 2: 28-33; and Ke and Madison, 1997, Nucleic Acids Res. 25: 3371-3372), repeated oligonucleotide-directed mutagenesis (Reidhaar-Olson et al., 1991, Methods Enzymol. 208:564-586), and any of the aforementioned methods of random mutagenesis.

Another method of creating diversity upon which directed evolution can act is the use of mutator genes. The nucleic acid of interest is cultured in a mutator cell strain the genome that encodes defective DNA repair genes (U.S. Pat. No. 6,365,410; Selifonova et al., 2001, Appl. Environ. Microbiol. 67:3645-3649; Long-McGie et al., 2000, Biotech. Bioeng. 68:121-125; see, Genencor International Inc, Palo Alto Calif.).

Achieving diversity using directed evolution techniques may also be accomplished using saturation mutagenesis along with degenerate primers (Gene Site Saturation Mutagenesis™, Diversa Corp., San Diego, Calif.). In this type of saturation mutagenesis, degenerate primers designed to cover the length of the nucleic acid sequence to be diversified are used to prime the polymerase in PCR reactions. In this manner, each codon of a coding sequence for an amino acid may be mutated to encode each of the remaining common nineteen amino acids. This technique may also be used to introduce mutations, deletions and insertions to specific regions of a nucleic acid coding sequence while leaving the rest of the nucleic acid molecule untouched. Procedures in the art may be used for the gene saturation technique, which can be found in U.S. Pat. No. 6,171,820.

b. DNA Shuffling

Botulinum neurotoxin proteins may also be generated using the techniques of gene-shuffling, motif-shuffling, exon-shuffling, and/or codon-shuffling (collectively referred to as “DNA shuffling”). DNA shuffling techniques may be employed to modulate the activities of peptides and may be used to generate peptides having altered activity. See, generally, U.S. Pat. Nos. 5,605,793; 5,811,238; 5,830,721; 5,834,252; and 5,837,458, and Stemmer et al. (1994, Nature 370(6488):389-391); Crameri et al. (1998, Nature 391 (6664):288-291); Zhang et al. (1997, Proc. Natl. Acad. Sci. USA 94(9):4504-4509); Stemmer et al. (1994, Proc. Natl. Acad. Sci USA 91(22):10747-10751), Patten et al. (1997, Curr. Opinion Biotechnol. 8:724-33); Harayama, (1998, Trends Biotechnol. 16(2):76-82); Hansson, et al., (1999, J. Mol. Biol. 287:265-76); and Lorenzo and Blasco (1998, Biotechniques 24(2):308-13) (each of these patents are hereby incorporated by reference in its entirety).

DNA shuffling involves the assembly of two or more DNA segments by homologous or site-specific recombination to generate variation in the polynucleotide sequence. DNA shuffling has been used to generate novel variations of human immunodeficiency virus type 1 proteins (Pekrun et al., 2002, J. Virol. 76(6):2924-35), triazine hydrolases (Raillard et al. 2001, Chem Biol 8(9):891-898), murine leukemia virus (MLV) proteins (Powell et al. 2000, Nat Biotechnol 18(12):1279-1282), and indoleglycerol phosphate synthase (Merz et al. 2000, Biochemistry 39(5):880-889).

The technique of DNA shuffling was developed to generate biomolecular diversity by mimicking natural recombination by allowing in vitro homologous recombination of DNA (Stemmler, 1994, Nature 370: 389-391; and Stemmler, 1994, PNAS 91: 10747-10751). Generally, in this method a population of related genes is fragmented and subjected to recursive cycles of denaturation, rehybridization, followed by the extension of the 5′ overhangs by Taq polymerase. With each cycle, the length of the fragments increases, and DNA recombination occurs when fragments originating from different genes hybridize to each other. The initial fragmentation of the DNA is usually accomplished by nuclease digestion, such as with DNase (see Stemmler references, above), but may also be accomplished by interrupted PCR synthesis (U.S. Pat. No. 5,965,408, incorporated herein by reference in its entirety; see, Diversa Corp., San Diego, Calif). DNA shuffling methods have advantages over random point mutation methods in that direct recombination of beneficial mutations generated by each round of shuffling is achieved and there is therefore a self selection for improved phenotypes of peptides.

The techniques of DNA shuffling are well known to those in art. Detailed explanations of such technology is found in Stemmler, 1994, Nature 370: 389-391 and Stemmler, 1994, PNAS 91: 10747-10751. The DNA shuffling technique is also described in U.S. Pat. Nos. 6,180,406, 6,165,793, 6,132,970, 6,117,679, 6,096,548, 5,837,458, 5,834,252, 5,830,721, 5,811,238, and 5,605,793 (all of which are incorporated by reference herein in their entirety).

The art also provides even more recent modifications of the basic technique of DNA shuffling. In one example, exon shuffling, exons or combinations of exons that encode specific domains of peptides are amplified using chimeric oligonucleotides. The amplified molecules are then recombined by self-priming PCR assembly (Kolkman and Stemmler, 2001, Nat. Biotech. 19:423-428). In another example, using the technique of random chimeragenesis on transient templates (RACHITT) library construction, single stranded parental DNA fragments are annealed onto a full-length single-stranded template (Coco et al., 2001, Nat. Biotechnol. 19:354-359). In yet another example, staggered extension process (StEP), thermocycling with abbreviated annealing/extension cycles is employed to repeatedly interrupt DNA polymerization from flanking primers (Zhao et al., 1998, Nat. Biotechnol. 16: 258-261). In the technique known as CLERY, in vitro family shuffling is combined with in vivo homologous recombination in yeast (Abecassis et al., 2000, Nucleic Acids Res. 28:E88). To maximize intergenic recombination, single stranded DNA from complementary strands of each of the nucleic acids are digested with DNase and annealed (Kikuchi et al., 2000, Gene 243:133-137). The blunt ends of two truncated nucleic acids of variable lengths that are linked by a cleavable sequence are then ligated to generate gene fusion without homologous recombination (Sieber et al., 2001, Nat Biotechnol. 19:456-460; Lutz et al., 2001, Nucleic Acids Res. 29:E16; Ostermeier et al., 1999, Nat. Biotechnol. 17:1205-1209; Lutz and Benkovic, 2000, Curr. Opin. Biotechnol. 11:319-324). Recombination between nucleic acids with little sequence homology in common has also been enhanced using exonuclease-mediated blunt-ending of DNA fragments and ligating the fragments together to recombine them (U.S. Pat. No. 6,361,974, incorporated herein by reference in its entirety). Each and every variation described above is contemplated for enhancing the biological properties of any of the peptides and/or enzymes described herein.

In addition to published protocols detailing directed evolution and gene shuffling techniques, commercial services are now available that will undertake the gene shuffling and selection procedures on peptides of choice. Maxygen (Redwood City, Calif.) offers commercial services to generate custom DNA shuffled libraries. In addition, this company will perform customized directed evolution procedures including gene shuffling and selection on a peptide family of choice.

Optigenix, Inc. (Newark, Del.) offers the related service of plasmid shuffling. Optigenix uses families of genes to obtain mutants therein having new properties. The nucleic acid of interest is cloned into a plasmid in an Aspergillus expression system. The DNA of the related family is then introduced into the expression system and recombination in conserved regions of the family occurs in the host. Resulting mutant DNAs are then expressed and the peptide produced therefrom are screened for the presence of desired properties and the absence of undesired properties.

c. Screening Procedures

Following each recursive round of “evolution,” the desired proteins or peptides expressed by mutated genes are screened for characteristics of interest. The “candidate” genes are then amplified and pooled for the next round of DNA shuffling. The screening procedure used is highly dependent on the peptide that is being “evolved” and the characteristic of interest. Characteristics such as peptide stability, biological activity, antigenicity, among others can be selected. Individual assays for the biological activity of peptides described herein.

d. Combinations of Techniques

It will be appreciated by the skilled artisan that the above techniques of mutation and selection can be combined with each other and with additional procedures to generate the peptides described herein. Thus, methods for the generation of peptides is not limited to one technique, but encompassed any and all of the methodology described herein. For example, a procedure for introducing point mutations into a nucleic acid sequence may be performed initially, followed by recursive rounds of DNA shuffling, selection and amplification. The initial introduction of point mutations may be used to introduce diversity into a gene population where it is lacking, and the following round of DNA shuffling and screening will select and recombine advantageous point mutations.

### In Vitro and In Vivo Expression Systems

### A. Cells for the Production of Peptides or Proteins

A discussion of several cell systems is now presented which establishes the power of the present methods and its independence of the cell type and systems in which a peptide or protein, such as an LC/A protease, can be produced.

In general, to express a peptide from a nucleic acid encoding it, the nucleic acid must be incorporated into an expression cassette, comprising a promoter element, a terminator element, and the coding sequence of the peptide operably linked between the two. The expression cassette is then operably linked into a vector. Toward this end, adapters or linkers may be employed to join the nucleotide fragments or other manipulations may be involved to provide for convenient restriction sites, removal of superfluous nucleotides, removal of restriction sites, or the like. For this purpose, in vitro mutagenesis, primer repair, restriction, annealing, re-substitutions, e.g., transitions and transversions, may be involved. A shuttle vector has the genetic elements necessary for replication in a cell. Some vectors may be replicated only in prokaryotes, or may be replicated in both prokaryotes and eukaryotes. Such a plasmid expression vector will be maintained in one or more replication systems, for example in two replication systems that allow for stable maintenance within a yeast host cell for expression purposes, and within a prokaryotic host for cloning purposes. Many vectors with diverse characteristics are now available commercially. Vectors are usually plasmids or phages, but may also be cosmids or mini-chromosomes. Conveniently, many commercially available vectors will have the promoter and terminator of the expression cassette already present, and a multi-linker site where the coding sequence for the peptide of interest can be inserted. The shuttle vector containing the expression cassette is then transformed in E. coli where it is replicated during cell division to generate a preparation of vector that is sufficient to transform the host cells of the chosen expression system. Such protocols can be found in Sambrook et al. (2001, Molecular Cloning: A Laboratory Manual, Cold Spring Harbor Laboratory, New York).

The vector, once purified from the cells in which it is amplified, is then transformed into the cells of the expression system. The protocol for transformation depended on the kind of the cell and the nature of the vector. Transformants are grown in an appropriate nutrient medium, and, where appropriate, maintained under specific pressure to insure retention of endogenous DNA. Where expression is inducible, growth can be permitted of the yeast host to yield a high density of cells, and then expression is induced. The secreted, mature heterologous peptide can be harvested by any means, and purified by chromatography, electrophoresis, dialysis, solvent-solvent extraction, and the like.

The techniques in the art may be used for molecular cloning. Further, techniques for the procedures of molecular cloning can be found in Sambrook et al. (2001, Molecular Cloning: A Laboratory Manual, Cold Spring Harbor Laboratory Press, Cold Spring Harbor, N.Y.); Glover et al., (1985, DNA Cloning: A Practical Approach, Volumes I and II); Gait et al., (1985, Oligonucleotide Synthesis); Hames and Higgins (1985, Nucleic Acid Hybridization); Hames and Higgins (1984, Transcription And Translation); Freshney et al., (1986, Animal Cell Culture); Perbal, (1986, Immobilized Cells And Enzymes, IRL Press); Perbal, (1984, A Practical Guide To Molecular Cloning); Ausubel et al. (2002, Current Protocols in Molecular Biology, John Wiley & Sons, Inc.).

### B. Fungi and Yeast

Peptides may be produced in yeast. By “yeast” is intended ascosporogenous yeasts (Endomycetales), basidiosporogenous yeasts, and yeast belonging to the Fungi Imperfecti (Blastomycetes). The ascosporogenous yeasts are divided into two families, Spermophthoraceae and Saccharomycetaceae. The later is comprised of four subfamilies, Schizosaccharomycoideae (e.g., genus Schizosaccharomyces), Nadsonioideae, Lipomycoideae, and Saccharomycoideae (e.g., genera Pichia, Kluyveromyces, and Saccharomyces). The basidiosporogenous yeasts include the genera Leucosporidium, Rhodosporidium, Sporidiobolus, Filobasidium, and Filobasidiella. Yeast belonging to the Fungi Imperfecti are divided into two families, Sporobolomycetaceae (e.g., genera Sporobolomyces, Bullera) and Cryptococcaceae (e.g., genus Candida). Of particular interest are species within the genera Saccharomyces, Pichia, Aspergillus, Trichodernia, Kluyveromyces, especially K. lactis and K. drosophilum, Candida, Hansenula, Schizpsaccaromyces, Yarrowia, and Chrysoporium. Since the classification of yeast may change in the future, yeast may be defined as described in Skinner et al., eds. 1980) Biology and Activities of Yeast (Soc. App. Bacteriol. Symp. Series No. 9).

In addition to the foregoing, methods in the art may be used for manipulation of yeast genetics. See, for example, Bacila et al., eds. (1978, Biochemistry and Genetics of Yeast, Academic Press, New York); and Rose and Harrison. (1987, The Yeasts (2.sup.nd ed.) Academic Press, London). Methods in the art may be used to for introducing exogenous DNA into yeast hosts. There are a wide variety of methods for transformation of yeast. Spheroplast transformation is taught by Hinnen et al (1978, Proc. Natl. Acad. Sci. USA 75:1919-1933); Beggs, (1978, Nature 275(5676):104-109); and Stinchcomb et al., (EPO Publication No. 45,573; herein incorporated by reference), Electroporation is taught by Becker and Gaurante, (1991, Methods Enzymol. 194:182-187), Lithium acetate is taught by Gietz et al. (2002, Methods Enzymol. 350:87-96) and Mount et al. (1996, Methods Mol Biol. 53:139-145). For a review of transformation systems of non-Saccharomyces yeasts, see Wang et al. (Crit Rev Biotechnol. 2001; 21(3):177-218). For general procedures on yeast genetic engineering, see Barr et al., (1989, Yeast genetic engineering, Butterworths, Boston).

In addition to wild-type yeast and fungal cells, there are also strains of yeast and fungi that have been mutated and/or selected to enhance the level of expression of the exogenous gene, and the purity, the post-translational processing of the resulting peptide, and the recovery and purity of the mature peptide. Expression of an exogenous peptide may also be direct to the cell secretory pathway, as illustrated by the expression of insulin (see (Kjeldsen, 2000, Appl. Microbiol. Biotechnol. 54:277-286, and references cited therein). In general, to cause the exogenous peptide to be secreted from the yeast cell, secretion signals derived from yeast genes may be used, such as those of the genes of the killer toxin (Stark and Boyd, 1986, EMBO J. 5:1995-2002) or of the alpha pheromone (Kurjan and Herskowitz, 1982, Cell 30:933; Brake et al., 1988, Yeast 4:S436).

Regarding the filamentous fungi in general, methods for genetic manipulation can be found in Kinghorn and Turner (1992, Applied Molecular Genetics of Filamentous Fungi, Blackie Academic and Professional, New York). Guidance on appropriate vectors can be found in Martinelli and Kinghorn (1994, Aspergillus: 50 years, Elsevier, Amsterdam). 1. Saccharomyces

In Saccharomyces, suitable yeast vectors for use producing a peptide include YRp7 (Struhl et al., Proc. Natl. Acad. Sci. USA 76: 1035-1039, 1978), YEp13 (Broach et al., Gene 8: 121-133, 1979), POT vectors (Kawasaki et al, U.S. Pat. No. 4,931,373, which is incorporated by reference herein), pJDB249 and pJDB219 (Beggs, Nature 275:104-108, 1978) and derivatives thereof. Promoters for use in yeast include promoters for yeast glycolytic gene expression (Hitzeman et al., J. Biol. Chem. 255: 12073-12080, 1980; Alber and Kawasaki, J. Mol. Appl. Genet. 1: 419-434, 1982; Kawasaki, U.S. Pat. No. 4,599,311) or alcohol dehydrogenase genes (Young et al., in Genetic Engineering of Microorganisms for Chemicals, Hollaender et al., (eds.), p. 355, Plenum, New York, 1982; Ammerer, Meth. Enzymol. 101: 192-201, 1983), and the ADH2-4.sup.c promoter (Russell et al., Nature 304: 652-654, 1983; Irani and Kilgore, U.S. patent application Ser. No. 07/784,653, CA 1,304,020 and EP 284 044, which are incorporated herein by reference). The expression units may also include a transcriptional terminator. A transcriptional terminator is the TPI1 terminator (Alber and Kawasaki, ibid.).

Examples of such yeast-bacteria shuttle vectors include Yep24 (Botstein et al. (1979) Gene 8:17-24; pC1 (Brake et al. (1984) Proc. Natl. Acad. Sci. USA 81:4642-4646), and Yrp17 (Stnichomb et al. (1982) J. Mol. Biol. 158:157). Additionally, a plasmid expression vector may be a high or low copy number plasmid, the copy number generally ranging from about 1 to about 200. In the case of high copy number yeast vectors, there will generally be at least 10, at least 20, and usually not exceeding about 150 copies of the vector in a single host. Depending upon the heterologous peptide selected, either a high or low copy number vector may be desirable, depending upon the effect of the vector and the recombinant peptide on the host. See, for example, Brake et al. (1984) Proc. Natl. Acad. Sci. USA 81:4642-4646. DNA constructs can also be integrated into the yeast genome by an integrating vector. Examples of such vectors in the art may be used herein. See, for example, Botstein et al. (1979) Gene 8:17-24.

The selection of suitable yeast and other microorganism hosts is within the skill of the art. Of particular interest are the Saccharomyces species S. cerevisiae, S. carlsbergenisis, S. diastaticus, S. douglasii, S. kluyveri, S. norbensis, and S. oviformis. When selecting yeast host cells for expression of a desired peptide, suitable host cells may include those shown to have, inter alia, good secretion capacity, low proteolytic activity, and overall vigor. Yeast and other microorganisms are generally available from a variety of sources, including the Yeast Genetic Stock Center, Department of Biophysics and Medical Physics, University of California, Berkeley, Calif.; and the American Type Culture Collection, Manassas Va. For a review, see Strathem et al., eds. (1981, The Molecular Biology of the Yeast Saccharomyces, Cold Spring Harbor Laboratory, Cold Spring Harbor, N.Y.). Methods in the art may be used for introducing exogenous DNA into yeast hosts.

### 2. Pichia

The use of Pichia methanolica as a host cell for the production of recombinant peptides is disclosed in PCT Applications WO 97/17450, WO 97/17451, WO 98/02536, and WO 98/02565. DNA molecules for use in transforming P. methanolica are commonly prepared as double-stranded, circular plasmids, which may be linearized prior to transformation. For peptide production in P. methanolica, the promoter and terminator in the plasmid may be that of a P. methanolica gene, such as a P. methanolica alcohol utilization gene (AUG1 or AUG2). Other useful promoters include those of the dihydroxyacetone synthase (DHAS), formate dehydrogenase (FMD), and catalase (CAT) genes, as well as those disclosed in U.S. Pat. No. 5,252,726. To facilitate integration of the DNA into the host chromosome, the entire expression segment of the plasmid may be flanked at both ends by host DNA sequences. A selectable marker for use in Pichia methanolica is a P. methanolica ADE2 gene, which encodes phosphoribosyl-5-aminoimidazole carboxylase (AIRC; EC 4.1.1.21), which allows ade2 host cells to grow in the absence of adenine. For large-scale, industrial processes where it is desirable to minimize the use of methanol, host cells in which both methanol utilization genes (AUG1 and AUG2) are deleted may be used. For production of secreted peptides, host cells deficient in vacuolar protease genes (PEP4 and PRB1) may be used. Electroporation is used to facilitate the introduction of a plasmid containing DNA encoding a peptide of interest into P. methanolica cells. P. methanolica cells may be transformed by electroporation using an exponentially decaying, pulsed electric field having a field strength of from 2.5 to 4.5 kV/cm, about 3.75 kV/cm, and a time constant (t) of from 1 to 40 milliseconds, or about 20 milliseconds. For a review of the use of Pichia pastoris for large-scale production of antibody fragments, see Fischer et al., (1999, Biotechnol Appl Biochem. 30 (Pt 2):117-120).

### 3. Aspergillus

Methods in the art may be used to express peptides in Aspergillus spp., including but not limited to those described in Carrez et al., 1990, Gene 94:147-154; Contreras, 1991, Bio/Technology 9:378-381; Yelton et al., 1984, Proc. Natl. Acad. Sci. USA 81:1470-1474; Tilbum et al., 1983, Gene 26:205-221; Kelly and. Hynes, 1985, EMBO J. 4:475-479; Ballance et al., 1983, Biochem. Biophys. Res. Comm. 112:284-289; Buxton et al., 1985, Gene 37:207-214, and U.S. Pat. No. 4,935,349, incorporated by reference herein in its entirety. Examples of promoters useful in Aspergillus are found in U.S. Pat. No. 5,252,726. Strains of Aspergillus useful for peptide expression are found in U.S. Pat. No. 4,935,349. Commercial production of exogenous peptides is available from Novoenzymes for Aspergillus niger and Aspergillus oryzae.

### 4. Trichoderma

Trichoderma species useful as hosts for the production of peptides to be remodeled include T. reesei, such as QM6a, ALK02442 or CBS383.78 (Centraalbureau voor Schimmelcultures, Oosterstraat 1, PO Box 273, 3740 AG Baarn, The Netherlands, or, ATCC13631 (American Type Culture Collection, Manassas Va., 10852, USA, type); T viride (such as CBS 189.79 (det. W. Gams); T longibrachiatum, such as CBS816.68 (type); T. pseudokoningii (such as MUCL19358; Mycotheque de l'Universite Catholique de Louvain); T. satunisporum CBS330.70 (type); T. harzianum CBS316.31 (det. W. Gams); T virgatum (T. pseudokoningii) ATCC24961. The host may be T reesei, such as T reesei strains QM9414 (ATCC 26921), RUT-C-30 (ATCC 56765), and highly productive mutants such as VTT-D-79125, which is derived from QM9414 (Nevalainen, Technical Research Centre of Finland Publications 26, (1985), Espoo, Finland).

Methods in the art may be used for the transformation of Trichoderma with DNA, including that taught in European patent No. EP0244234, Harkki (1989, Bio/Technology 7:596-601) and Uusitalo (1991, J. Biotech. 17:35-50). Culture of Trichoderma is supported by previous extensive experience in industrial scale fermentation techniques; for example, see Finkelstein, 1992, Biotechnology of Filamentous Fungi: Technology and Products, Butterworth-Heinemann, publishers, Stoneham, Mass.

### 5. Kluyveromyces

Yeast belonging to the genus Kluyveromyces have been used as host organisms for the production of recombinant peptides. Peptides produced by this genus of yeast are, in particular, chymosin (European Patent 96 430), thaumatin (European Patent 96 910), albumin, interleukin-1.beta., TPA, TIMP (European Patent 361 991) and albumin derivatives having a therapeutic function (European Patent 413 622). Species of particular interest in the genus Kluyveromyces include K lactis.

Methods in the art may be used for expressing recombinant peptides in Kluyvermyces spp. Vectors in the art may be used for the expression and secretion of human recombinant peptides in Kluyvermyces (Yeh, J. Cell. Biochem. Suppl. 14C:68, Abst. H402; Fleer, 1990, Yeast 6 (Special Issue):5449) as are procedures for transformation and expression of recombinant peptides (Ito et al., 1983, J. Bacteriol. 153:163-168; van den Berg, 1990, Bio/Technology 8:135-139; U.S. Pat. No. 5,633,146, WO8304050A1, EP0096910, EP0241435, EP0301670, EP0361991, all of which are incorporated by reference herein in their entirety). For a review of genetic manipulation of Kluyveromyces lactis linear DNA plasmids by gene targeting and plasmid shuffles, see Schaffrath et al. (1999, FEMS Microbiol Lett. 178(2):201-210).

### 6. Chrysoporium

The fungal genus Chrysoporium has recently been used to expression of foreign recombinant peptides. A description of the procedures on how Chrysoporium can be used to express foreign peptides is found in WO 00/20555 (incorporated by reference herein in its entirety). Species particularly suitable for expression system include, but are not limited to, C. botryoides, C. carmichaelii, C. crassitunicatum, C. europae, C. evokeannui, F. fastidium, C. filiforme, C. gerogiae, C. globiferum, C. globiferum var. articulatum, C. globiferum var. niveum, C. hirundo, C. hispanicum, C. holmii, C. indicum, C. thops, C. keratinophilum, C. kreiselii, C. kuzurovianum, C. lignorum, C. lobatum, C. lucknowense, C. lucknowense Garg 27K, C. medium, C. medium var. spissescens, C. mephiticum, C. merdarium, C. merdarium var. roseum, C. minor, C. pannicola, C. parvum, C. parvum var. crescens, C. pilosum, C. peodomerderium, C. pyrifonnis, C. queenslandicum, C. sigleri, C. sulfureum, C. synchronum, C. tropicum, C. undulatum, C. vallenarense, C. vespertilium, and C. zonatum.

Other Methods for transforming Schwanniomyces are disclosed in European Patent 394 538. Methods for transforming Acremonium chrysogenum are disclosed by U.S. Pat. No. 5,162,228. Methods for transforming Neurospora are disclosed by U.S. Pat. No. 4,486,533. Also know is an expression system specifically for Schizosaccharomyces pombe (European Patent 385 391). General methods for expressing peptides in fission yeast, Schizosaccharomyces pombe can be found in Giga-Hama and Kumagai (1997, Foreign gene expression in fission yeast: Schizosaccharomyces pombe, Springer, Berlin).

### C. Mammalian Systems

Using the methods described herein, a peptide or protein may be produced in a mammalian cell. Numerous expression vectors in the art may be useful for expressing exogenous peptides in mammalian cells. Many mammalian expression vectors are now commercially available from companies, including Novagen, Inc (Madison, Wis.), Gene Therapy Systems (San Diego, Calif.), Promega (Madison, Wis.), ClonTech Inc. (Palo Alto, Calif.), and Stratagene (La Jolla, Calif.), among others.

There are several mammalian cell lines that are particularly adept at expressing exogenous peptides. Mammalian cell lines may originate from tumor cells extracted from mammals that have become immortalized, that is to say, they can replicate in culture essentially indefinitely. These cell lines include, but are not limited to, CHO (Chinese hamster ovary, e.g., CHO-K1; ATCC No. CCL 61) and variants thereof, NS0 (mouse myeloma), BNK, BHK 570 (ATCC No. CRL 10314), BHK (ATCC No. CRL 1632), Per.C6™ (immortalized human cells, Crucell N. V., Leiden, The Netherlands), COS-1 (ATCC No. CRL 1650), COS-7 (ATCC No. CRL 1651), HEK 293, mouse L cells, T lymphoid cell lines, BW5147 cells and MDCK (Madin-Darby canine kidney), HeLa (human), A549 (human lung carcinoma), 293 (ATCC No. CRL 1573; Graham et al., 1977, Gen. Virol. 36:59-72), BGMK (Buffalo Green Monkey kidney), Hep-2 (human epidermoid larynx carcinoma), LLC-MK.sub.2 (African Green Monkey Kidney), McCoy, NCI-H292 (human pulmonary mucoepidermoid carcinoma tube), RD (rhabdomyosarcoma), Vero (African Green Monkey kidney), HEL (human embryonic lung), Human Fetal Lung-Chang, MRCS (human embryonic lung), MRHF (human foreskin), and WI-38 (human embryonic lung). In some cases, the cells in which the therapeutic peptide is expressed may be cells derived from the patient to be treated, or they may be derived from another related or unrelated mammal. For example, fibroblast cells may be isolated from the mammal's skin tissue, and cultured and transformed in vitro. This technology is commercially available from Transkaryotic Therapies, Inc. (Cambridge, Mass.). Almost all currently used cell lines are available from the American Type Culture Collection (ATCC, Manassas, Va.) and BioWhittaker (Walkersville, Md.).

Techniques in the art may be used to transform mammalian cells with DNA. Such techniques include, but are not limited to, calcium phosphate transformation (Chen and Okayama, 1988; Graham and van der Eb, 1973; Corsaro and Pearson, 1981, Somatic Cell Genetics 7:603), Diethylaminoethyl (DEAE)-dextran transfection (Fujita et al., 1986; Lopata et al., 1984; Selden et al., 1986), electroporation (Neumann et al., 1982; Potter, 1988; Potter et al., 1984; Wong and Neuman, 1982), cationic lipid reagent transfection (Elroy-Stein and Moss, 1990; Feigner et al., 1987; Rose et al., 1991; Whitt et al., 1990; Hawley-Nelson et al., 1993, Focus 15:73; Ciccarone et al., 1993, Focus 15:80), retroviral (Cepko et al., 1984; Miller and Baltimore, 1986; Pear et al., 1993; Austin and Cepko, 1990; Bodine et al., 1991; Fekete and Cepko, 1993; Lemischka et al., 1986; Turner et al., 1990; Williams et al., 1984; Miller and Rosman, 1989, BioTechniques 7:980-90; Wang and Finer, 1996, Nature Med. 2:714-6), polybrene (Chaney et al, 1986; Kawai and Nishizawa, 1984), microinjection (Capecchi, 1980), and protoplast fusion (Rassoulzadegan et al., 1982; Sandri-Goldin et al., 1981; Schaffer, 1980), among others. In general, see Sambrook et al. (2001, Molecular Cloning: A Laboratory Manual, Cold Spring Harbor Laboratory, New York) and Ausubel et al. (2002, Current Protocols in Molecular Biology, John Wiley & Sons, New York) for transformation techniques.

Recently the baculovirus system, popular for transformation of insect cells, has been adapted for stable transformation of mammalian cells (see, for review, Koat and Condreay, 2002, Trends Biotechnol. 20:173-180, and references cited therein). The production of recombinant peptides in cultured mammalian cells is disclosed, for example, in U.S. Pat. Nos. 4,713,339, 4,784,950; 4,579,821; and 4,656,134. Several companies offer the services of transformation and culture of mammalian cells, including Cell Trends, Inc. (Middletown, Md.). Techniques in the art may be used for culturing mammalian cells, and are further found in Hauser et al. (Mammalian Cell Biotechnology, Walter de Gruyer, Inc., Hawthorne, N.Y., 1997) and Sambrook et al. (2001, Molecular Cloning: A Laboratory Manual, Cold Spring Harbor and references cited therein.

### D. Insect

Insect cells and in particular, cultured insect cells, maybe used for recombinant peptide production. Baculovirus-mediated expression in insect cells has become particularly well-established for the production of recombinant peptides (Altmann et al., 1999, Glycoconjugate J. 16:109-123). With regard to peptide folding and post-translational processing, insect cells are second only to mammalian cell lines.

Protocols in the art may be incorporated for the use of baculovirus to transform insect cells. Several books have been published which provide the procedures to use the baculovirus system to express peptides in insect cells. These books include, but are not limited to, Richardson (Baculovirus Expression Protocols, 1998, Methods in Molecular Biology, Vol 39, Humana Pr), O'Reilly et al. (1994, Baculovirus Expression Vectors: A Laboratory Manual, Oxford Univ Press), and King and Possee (1992, The Baculovirus Expression System: A Laboratory Guide, Chapman & Hall). In addition, there are also publications such as Lucklow (1993, Curr. Opin. Biotechnol. 4:564-572) and Miller (1993, Curr. Opin. Genet. Dev. 3:97-101).

Many patents have also been issued that related to systems for baculoviral expression of foreign proteins. These patents include, but are not limited to, U.S. Pat. No. 6,210,966 (Culture medium for insect cells lacking glutamine and containing ammonium salt), U.S. Pat. No. 6,090,584 (Use of BVACs (BaculoVirus Artificial Chromosomes) to produce recombinant peptides), U.S. Pat. No. 5,871,986 (Use of a baculovirus to express a recombinant nucleic acid in a mammalian cell), U.S. Pat. No. 5,759,809 (Methods of expressing peptides in insect cells and methods of killing insects), U.S. Pat. No. 5,753,220 (Cysteine protease gene defective baculovirus, process for its production, and process for the production of economic peptide by using the same), U.S. Pat. No. 5,750,383 (Baculovirus cloning system), U.S. Pat. No. 5,731,182 (Non-mammalian DNA virus to express a recombinant nucleic acid in a mammalian cell), U.S. Pat. No. 5,728,580 (Methods and culture media for inducing single cell suspension in insect cell lines), U.S. Pat. No. 5,583,023 (Modified baculovirus, its preparation process and its application as a gene expression vector), U.S. Pat. No. 5,571,709 (Modified baculovirus and baculovirus expression vectors), U.S. Pat. No. 5,521,299 (Oligonucleotides for detection of baculovirus infection), U.S. Pat. No. 5,516,657 (Baculovirus vectors for expression of secretory and membrane-bound peptides), U.S. Pat. No. 5,475,090 (Gene encoding a peptide which enhances virus infection of host insects), U.S. Pat. No. 5,472,858 (Production of recombinant peptides in insect larvae), U.S. Pat. No. 5,348,886 (Method of producing recombinant eukaryotic viruses in bacteria), U.S. Pat. No. 5,322,774 (Prokaryotic leader sequence in recombinant baculovirus expression system), U.S. Pat. No. 5,278,050 (Method to improve the efficiency of processing and secretion of recombinant genes in insect systems), U.S. Pat. No. 5,244,805 (Baculovirus expression vectors), U.S. Pat. No. 5,229,293 (Recombinant baculovirus), U.S. Pat. No. 5,194,376 (Baculovirus expression system capable of producing recombinant peptides at high levels), U.S. Pat. No. 5,179,007 (Method and vector for the purification of recombinant peptides), U.S. Pat. No. 5,169,784 (Baculovirus dual promoter expression vector), U.S. Pat. No. 5,162,222 (Use of baculovirus early promoters for expression of recombinant nucleic acids in stably transformed insect cells or recombinant baculoviruses), U.S. Pat. No. 5,155,037 (Insect signal sequences useful to improve the efficiency of processing and secretion of recombinant nucleic acids in insect systems), U.S. Pat. No. 5,147,788 (Baculovirus vectors and methods of use), U.S. Pat. No. 5,110,729 (Method of producing peptides using baculovirus vectors in cultured cells), U.S. Pat. No. 5,077,214 (Use of baculovirus early promoters for expression of recombinant genes in stably transformed insect cells), U.S. Pat. No. 5,023,328 (Lepidopteran AKH signal sequence), and U.S. Pat. Nos. 4,879,236 and 4,745,051 (Method for producing a recombinant baculovirus expression vector). All of the aforementioned patents are incorporated in their entirety by reference herein.

Insect cell lines in the art of several different species origin may be used for peptide expression. Insect cell lines of interest include, but are not limited to, dipteran and lepidopteran insect cells in general, Sf9 and variants thereof (fall armyworm Spodoptera frugiperda), Estigmene acrea, Trichoplusia ni, Bombyx mori, Malacosoma disstri. drosophila lines Kc1 and SL2 among others, and mosquito.

### E. Plants

Following the directions provided herein, it is now possible to generate a peptide produced in a plant cell. Transgenic plants are considered by many to be the expression system of choice for pharmaceutical peptides. Potentially, plants can provide a cheaper source of recombinant peptides. It has been estimated that the production costs of recombinant peptides in plants could be between 10 to 50 times lower than that of producing the same peptide in E. coli. While there are slight differences in the codon usage in plants as compared to animals, these can be compensated for by adjusting the recombinant DNA sequences (see, Kusnadi et al., 1997, Biotechnol. Bioeng. 56:473-484; Khoudi et al., 1999, Biotechnol. Bioeng. 135-143; Hood et al., 1999, Adv. Exp. Med. Biol. 464:127-147). In addition, peptide synthesis, secretion and post-translational modification are very similar in plants and animals, with only minor differences in plant glycosylation (see, Fischer et al., 2000, J. Biol. Regul. Homest. Agents 14: 83-92). Then, products from transgenic plants are also less likely to be contaminated by animal pathogens, microbial toxins and oncogenic sequences.

Methods in the art may be used for expression of recombinant peptides in plant cells. In addition to transgenic plants, peptides can also be produced in transgenic plant cell cultures (Lee et al., 1997, Mol. Cell. 7:783-787), and non-transgenic plants inoculated with recombinant plant viruses. Several books have been published that describe protocols for the genetic transformation of plant cells: Potrykus (1995, Gene transfer to plants, Springer, New York), Nickoloff (1995, Plant cell electroporation and electrofusion protocols, Humana Press, Totowa, N.Y.) and Draper (1988, Plant genetic transformation, Oxford Press, Boston).

Several methods are currently used to stably transform plant cells with recombinant genetic material. These methods include, but are not limited to, Agrobacterium transformation (Bechtold and Pelletier, 1998; Escudero and Hohn, 1997; Hansen and Chilton, 1999; Touraev et al., 1997), biolistics (microprojectiles) (Finer et al., 1999; Hansen and Chilton, 1999; Shilito, 1999), electroporation of protoplasts (Fromm et al., 1985, Ou-Lee et al., 1986; Rhodes et al., 1988; Saunders et al., 1989; Trick et al., 1997), polyethylene glycol treatment (Shilito, 1999; Trick et al., 1997), in planta mircroinjection (Leduc et al., 1996; Zhou et al., 1983), seed imbibition (Trick et al., 1997), laser beam (1996), and silicon carbide whiskers (Thompson et al., 1995; U.S. Patent Appln. No. 20020100077, incorporated by reference herein in its entirety).

Many kinds of plants are amenable to transformation and expression of exogenous peptides. Plants of particular interest to express the peptides include, but are not limited to, Arabidopsis thalliana, rapeseed (Brassica spp.; Ruiz and Blumwald, 2002, Planta 214:965-969)), soybean (Glycine max), sunflower (Helianthus unnuus), oil palm (Elaeis guineeis), groundnut (peanut, Arachis hypogaea; Deng et al., 2001, Cell. Res. 11:156-160), coconut (Cocus nucifera), castor (Ricinus communis), safflower (Carthamus tinctorius), mustard (Brassica spp. and Sinapis alba), coriander, (Coriandrum sativum), squash (Cucurbita maxima; Spencer and Snow, 2001, Heredity 86(Pt 6):694-702), linseed/flax (Linum usitatissimum; Lamblin et al., 2001, Physiol Plant 112:223-232), Brazil nut (Bertholletia excelsa), jojoba (Simmondsia chinensis), maize (Zea mays; Hood et al., 1999, Adv. Exp. Med. Biol. 464:127-147; Hood et al., 1997, Mol. Breed. 3:291-306; Petolino et al., 2000, Transgenic Research 9:1-9), alfalfa (Khoudi et al., 1999, Biotechnol. Bioeng. 64:135-143), tobacco (Nicotiana tabacum; Wright et al., Transgenic Res. 10:177-181; Frigerio et al., 2000, Plant Physiol. 123:1483-1493; Crameret al., 1996, Ann. New York Acad. Sci. 792:62-8-71; Cabanes-Macheteau et al., 1999, Glycobiology 9:365-372; Ruggiero et al., 2000, FEBS Lett. 469:132-136), canola (Bai et al., 2001, Biotechnol. Prog. 17:168-174; Zhang et al., 2000, J. Anim. Sci. 78:2868-2878)), potato (Tacket et al., 1998, J. Infect. Dis. 182:302-305; Richter et al., 2000, Nat. Biotechnol. 18:1167-1171; Chong et al., 2000, Transgenic Res. 9:71-78), alfalfa (Wigdorovitz et al., 1999, Virology 255:347-353), Pea (Pisum sativum; Perrin et al., 2000, Mol. Breed. 6:345-352), rice (Oryza sativa; Stoger et al., 2000, Plant Mol. Biol. 42:583-590), cotton (Gossypium hirsutum; Kornyeyev et al., 2001, Physiol Plant 113:323-331), barley (Hordeum vulgare; Petersen et al., 2002, Plant Mol Biol 49:45-58); wheat (Triticum spp.; Pellegrineschi et al., 2002, Genome 45:421-430) and bean (Vicia spp.; Saalbach et al., 1994, Mol Gen Genet 242:226-236).

If expression of the recombinant nucleic acid is desired in a whole plant rather than in cultured cells, plant cells are first transformed with DNA encoding the peptide, following which, the plant is regenerated. This involves tissue culture procedures that may be optimized for each plant species. Protocols in the art for many plant species may be used to regenerate plants. Furthermore, protocols for other species can be developed by one of skill in the art using routine experimentation. Numerous laboratory manuals are available that describe procedures for plant regeneration, including but not limited to, Smith (2000, Plant tissue culture: techniques and experiments, Academic Press, San Diego), Bhojwani and Razdan (1996, Plant tissue culture: theory and practice, Elsevier Science Pub., Amsterdam), Islam (1996, Plant tissue culture, Oxford & IBH Pub. Co., New Delhi, India), Dodds and. Roberts (1995, Experiments in plant tissue culture, New York: Cambridge University Press, Cambridge England), Bhojwani (Plant tissue culture: applications and limitations, Elsevier, Amsterdam, 1990), Trigiano and Gray (2000, Plant tissue culture concepts and laboratory exercises, CRC Press, Boca Raton, Fla.), and Lindsey (1991, Plant tissue culture manual fundamentals and applications, Kluwer Academic, Boston).

While purifying recombinant peptides from plants may potentially be costly, several systems have been developed to minimize these costs. One method directs the synthesized peptide to the seed endosperm from where it can be easily extracted (Wright et al., 2001, Transgenic Res. 10:177-181, Guda et a., 2000, Plant Cell Res. 19:257-262; and U.S. Pat. No. 5,767,379, which is incorporated by reference herein in its entirety). An alternative approach is the co-extraction of the recombinant peptide with plant products such as starch, meal or oil. In oil-seed rape, a fusion peptide of oleosin-hurudin when expressed in the plant, attaches to the oil body of the seed, and can be extracted from the plant seed along with the oil (Parmenter, 1995, Plant Mol. Biol. 29:1167-1180; U.S. Pat. Nos. 5,650,554, 5,792,922, 5,948,682 and 6,288,304, and U.S. application 2002/0037303, all of which are incorporated in their entirely by reference herein). In a variation on this approach, the oleosin is fused to a peptide having affinity for the exogenous co-expressed peptide of interest (U.S. Pat. No. 5,856,452, incorporated by reference herein in its entirety).

For a general review on the technology for plastid expression of exogenous peptides in higher plants, see Hager and Beck (2000, Appl. Microbiol. Biotechnol. 54:302-310, and references cited therein). Plastid expression has been particularly successful in tobacco (see, for example, Staub et al., 2000, Nat. Biotechnol. 18:333-338).

### F. Transgenic Animals

Introduction of a recombinant DNA into the fertilized egg of an animal (e.g., a mammal) may be accomplished using any number of standard techniques in transgenic animal technology. See, e.g., Hogan et al., Manipulating the Mouse Embryo: A Laboratory Manual, Cold Spring Harbor Laboratory Press, Cold Spring Harbor, N.Y., 1986; and U.S. Pat. No. 5,811,634, which is incorporated by reference herein in its entirety. Most commonly, the recombinant DNA is introduced into the embryo by way of pronuclear microinjection (Gordon et al., 1980, PNAS 77:7380-7384; Gordon and Ruddle, 1981, Science 214:1244-1246; Brinster et al., 1981, Cell 27:223-231; Costantini and Lacy, 1981, Nature 294:92-94). Microinjection has the advantage of being applicable to a wide variety of species. Preimplantation embryos may also be transformed with retroviruses (Jaenisch and Mintz, 1974, Proc. Natl. Acad. Sci. U.S.A. 71:1250-1254; Jaenisch et al., 1976, Hamatol Bluttransfus. 19:341-356; Stuhlmann et al., 1984, Proc. Natl. Acad. Sci. U.S.A. 81:7151-7155). Retroviral mediated transformation has the advantage of adding single copies of the recombinant nucleic acid to the cell, but it produces a high degree of mosaicism. Most recently, embryonic stem cell-mediated techniques have been used (Gossler et al., 1986, Proc. Natl. Acad. Sci. U.S.A. 83:9065-9069), transfer of entire chromosomal segments (Lavitrano et al., 1989, Cell 57:717-723), and gamete transfection in conjunction with in vitro fertilization (Lavitrano et al., 1989, Cell 57:717-723) have also been used. Several books of laboratory procedures have been published disclosing these techniques: Cid-Arregui and Garcia-Carranca (1998, Microinjection and Transgenesis: Strategies and Protocols, Springer, Berlin), Clarke (2002, Transgenesis Techniques: Principles and Protocols, Humana Press, Totowa, N.J.), and Pinkert (1994, Transgenic Animal Technology: A Laboratory Handbook, Academic Press, San Diego).

Once the recombinant DNA is introduced into the egg, the egg is incubated for a short period of time and is then transferred into a pseudopregnant animal of the same species from which the egg was obtained (Hogan et al., supra). In the case of mammals, about 125 eggs may be injected per experiment, approximately two-thirds of which will survive the procedure. Twenty viable eggs are transferred into a pseudopregnant mammal, four to ten of which will develop into live progeny. In some cases, 10-30% of the progeny (in the case of mice) carry the recombinant DNA.

While the entire animal can be used as an expression system for the peptides, in some cases the exogenous peptide accumulates in products of the animal, from which it can be harvested without injury to the animal. For example, the exogenous peptide accumulates in milk, eggs, hair, blood, and urine.

If the recombinant peptide is to be accumulated in the milk of the animal, suitable mammals are ruminants, ungulates, domesticated mammals, and dairy animals. Exemplary animals are goats, sheep, camels, cows, pigs, horses, oxen, and llamas. Methods for generating transgenic cows that accumulate a recombinant peptide in their milk are well known: see, Newton (1999, J. Immunol. Methods 231:159-167), Ebert et al. (1991, Biotechnology 9: 835-838), and U.S. Pat. Nos. 6,210,736, 5,849,992, 5,843,705, 5,827,690, 6,222,094, all of which are incorporated herein by reference in their entirety. The generation of transgenic mammals that produce a desired recombinant peptide is commercially available from GTC Biotherapeutics, Framingham, Mass.

If the recombinant peptide is to be accumulated in eggs, suitable birds include, but are not limited to, chickens, geese, and turkeys. Other animals of interest include, but are not limited to, other species of avians, fish, reptiles and amphibians. Methods in the art may be used for the introduction of recombinant DNA to a chicken by retroviral transformation: Thoraval et al. (1995, Transgenic Research 4:369-376), Bosselman et al., (1989, Science 243: 533-535), Petropoulos et al. (1992, J. Virol. 66: 3391-3397), U.S. Pat. No. 5,162,215, incorporated by reference herein in its entirety. Successful transformation of chickens with recombinant DNA also been achieved wherein DNA is introduced into blastodermal cells and blastodermal cells so transfected are introduced into the embryo: Brazolot et al. (1991, Mol. Reprod. Dev. 30: 304-312), Fraser, et al. (1993, Int. J. Dev. Biol. 37: 381-385), and Petitte et al. (1990, Development 108: 185-189). High throughput technology has been developed to assess whether a transgenic chicken expresses the desired peptide (Harvey et al., 2002, Poult. Sci. 81:202-212, U.S. Pat. No. 6,423,488, incorporated by reference herein in its entirety). Using retroviral transformation of chicken with a recombinant DNA, exogenous beta-lactamase was accumulated in the egg white of the chicken (Harvey et al., 2002, Nat. Biotechnol. 20(4):396-399). The production of chickens producing exogenous peptides in egg is commercially available from AviGenics, Inc., Athens Ga.

### G. Bacteria

Numerous bacterial expression systems in the art may be used herein. Exemplary bacterial species include, but are not limited to, E. coli. and Bacillus species. Methods in the art for the expression of recombinant peptides in E. coli may be used herein. Protocols for E. coli-based expression systems are found in U.S. Appln No. 20020064835, U.S. Pat. Nos. 6,245,539, 5,606,031, 5,420,027, 5,151,511, and RE33,653, among others. Methods to transform bacteria include, but are not limited to, calcium chloride (Cohen et al., 1972, Proc. Natl. Acad. Sci. U.S.A. 69:2110-2114; Hanahan, 1983, J. Mol. Biol. 166:557-580; Mandel and Higa, 1970, J. Mol. Biol. 53:159-162) and electroporation (Shigekawa and Dower, 1988, Biotechniques 6:742-751), and those described in Sambrook et al., 2001 (supra). For a review of laboratory protocols on microbial transformation and expression systems, see Saunders and Saunders (1987, Microbial Genetics Applied to Biotechnology: Principles and Techniques of Gene Transfer and Manipulation, Croom Helm, London), Puhler (1993, Genetic Engineering of Microorganisms, Weinheim, New York), Lee et al., (1999, Metabolic Engineering, Marcel Dekker, New York), Adolph (1996, Microbial Genome Methods, CRC Press, Boca Raton), and Birren and Lai (1996, Nonmammalian Genomic Analysis: A Practical Guide, Academic Press, San Diego),

For a general review on the literature for peptide expression in E. coli see Balbas (2001, Mol. Biotechnol. 19:251-267). Several companies now offer bacterial strains selected for the expression of mammalian peptides, such as the Rosetta™ strains of E. coli (Novagen, inc., Madison, Wis.; with enhanced expression of eukaryotic codons not normally used in bacteria cells, and enhanced disulfide bond formation),

### H. Cell Engineering

A uniform starting material produced by a cell yields efficient generation in vitro of large quantities of peptides. Thus, the genetic engineering of host cells to produce peptides as starting material for the in vitro enzymatic reactions disclosed herein, provides a significant advantage. In general, any eukaryotic cell type can be modified to become a host cell.

The cell may be any type of cell and may be a eukaryotic cell. The cell may be a mammalian cell such as human, mouse, rat, rabbit, hamster or other type of mammalian cell. When the cell is a mammalian cell, the mammalian cell may be derived from or contained within a non-human transgenic mammal. In addition, the cell may be a fungal cell, a yeast cell, or the cell may be an insect or a plant cell. Similarly, when the cell is a plant cell, the plant cell may be derived from or contained within a transgenic.

The method herein is contemplated to include any and all such cells for the production of the proteins described herein.

### Example 2

### Exemplary Specificity of Engineered Bont Lc/a Protease Domains and Proteins

A DNA library of BoNT/A light chain (LC) protease (designated hereinafter as LC/A protease or LC protease) gene variants (or mutants) were constructed with oligonucleotides encoding the exemplary inventive substitutions in LC/A protease shown in Tables 3 and 4 for generating novel modified proteases that contribute to improved substrate specificity and catalysis. Mutagenesis strategies and methods utilized for generating the library are described in the sections above. Briefly, the modified LC/A proteases were engineered starting with a quadruple mutant of LC/A with the E148Y, K166F, S254A, and G305D substitutions (designated herein as protease variant of SEQ ID NO: 27 (WO2019/145577)) by screening more than 16 libraries and over 600 variants for improved specificity of SNAP23 over SNAP25. FIGS. 1A and 1B demonstrates the improved specificity of exemplary modified LC/A proteases for SNAP23 over SNAP25. In particular, FIG. 1A demonstrates improved specificity of exemplary modified SNAP23-specific LC/A proteases for SNAP23 over SNAP25 in LC/A assay buffer (50 mM HEPES pH 7.4). Further, FIG. 1B demonstrates improved specificity of two exemplary SNAP23-specific modified LC/A proteases for SNAP23 over SNAP25 in salt-containing intracellular buffer (50 mM KH₂PO₄ pH 7.4).

### Example 3

### Assay to Confirm Successful Display of Lc/a Protease on Bacteriophage

To confirm that an exemplary, active WT LC/A was successfully displayed on the P8 protein of M13 bacteriophage (LC/A Φ), affinity of LC/A Φ was compared to a recombinantly expressed and purified WT LC/A (rLC/A). Concentration of rLC/A and SNAP25 depolarization after resonance energy transfer (DARET) substrate was determined spectroscopically by A_{280 nm} signal using a 1 cm quartz cuvette and predicted molar absorbtivity (46800 M⁻¹ cm⁻¹ and 44800 M⁻¹ cm⁻¹ respectively). Concentration of LC/A Φ was determined spectroscopically by A_{268 nm}, A_{320 nm} signal using a 1 cm quartz cuvette and the following equation:

[nM phage]=(320 nm−268 nm)*8.31
Final concentration of rLC/A: 16.6 nM.
Final concentration of LC/A Φ: 10 nM.
Final concentrations of SNAP25 DARET substrate: 10.3, 5.1, 2.6, 1.3, 0.64, 0.32, 0.16, and 0.08 μM.

Depletion of substrate was detected by a fluorescence resonance energy transfer (FRET) assay, exciting BFP at 387 nm and detecting emission of GFP at 509 nm fluorescence units (FU). Initial rates (Vo) and substrate concentrations were fit to a Michaelis-Menten curve and Vmax and Km values were calculated using GraphPad prism. FIGS. 2A and 2B demonstrate that the exemplary active WT LC/A protease was successfully displayed on LC/A Φ as determined by comparing activity of rLC/A (shown in FIG. 2A) to LC/A Φ (shown in FIG. 2B). Vo refers to the initial rate of SNAP25 cleavage at various substrate concentrations, determined by monitoring changes in Fluorescence Units (FU) over time. Vₘₐₓ indicates the maximum rate of cleavage for each enzyme and Kₘ indicates the Michaelis-Menten constant. The assay outlined in this example can be employed on any LC/A protease described herein.

### Example 4

### ELISA to Detect Substrate Binding by Lc/a Protease

ELISA is an assay used for assessing protein-protein binding interactions. Briefly, ELISA was used to detect SNAP23 and SNAP25 substrate binding by WT LC/A phage. STOP4 phage with no displayed protein was used as a negative control for the assay. Plates were coated with 20 μg/mL SNAP23 or SNAP25 in 100 mM HEPES pH 7.2, 100 μL per well of a 96-well flat bottom NUNC immunoplate. Plates were shaken at 150 rpm at 4° C. for 18 h. Plates were blocked using 1×PBS pH 8.0 with 2 mg/mL casein, 400 μL per well for 30 min at 150 rpm. WT LC/A displayed on the P8 protein of M13 bacteriophage was diluted in 1×PBS pH 8.0 plus 2 mg/mL casein 0.01% (v/v) Tween (PCT). Plates were washed with PCT, 300 μL per well for 3 times. Plates were then incubated with WT LC/A phage for 1 hour at 150 rpm. Plates were washed as before with PCT and then incubated with a 1:5000 dilution of anti-M13-HRP antibody in PCT for 30 min at 150 rpm. Plates with anti-M13-HRP antibody were washed as before with PCT and then washed a final time with 1×PBS pH 8.0. Presence of HRP was detected with a solution of 0.02 g o-phenylenediamine dihydrochloride (OPD) dissolved in 100 μL hydrogen peroxide, and 10 mL citric acid buffer pH 5.0, 100 per well. Plates were developed in the dark for 90 minutes before being read for absorbance at 450 nm. Final concentrations of WT LC/A Phage: 20, 6.7, 2.2, and 0.73 nM. All conditions were performed in triplicate. FIG. 3A demonstrates SNAP23 and SNAP25 substrate binding by WT LC/A phage as determined by ELISA. FIG. 3B provides a schematic illustration of the complex formed by the binding of an anti-M13-HRP antibody, WT LC/A phage, and substrate. The assay outlined in this example can be employed on any LC/A protease, either phage-displayed or not, described herein.

### Example 5

### Protein Overexpression

Methods of expressing the LC/A proteases are provided herein. An exemplary LC/A protease gene variant library was transformed into BL21 (DE3) E. coli (One Shot™ Star™, ThermoFisher) cells and then spread onto LB/agar plates supplemented with 40 μg/mL kanamycin (LBₖₐₙ) for overnight incubation at 37° C. Single colonies were selected and inoculated single 96-well deep-well plate (DWP) seed cultures (300 μL LBₖₐₙ per well), then incubated at 37° C. with shaking at 225 rpm for 18 h. The protease variant of SEQ ID NO: 27 and an uninoculated culture were included as single wells on each DWP as positive and negative controls, respectively. An expression culture (630 μL LBₖₐₙ per well) was subsequently inoculated with 20 μL of the seed culture and incubated for 3 h at 37° C. with shaking at 225 rpm. To generate glycerol stocks, the cells remaining from the seed culture DWP were harvested via centrifugation (2056×g, 4° C., 1 h), then resuspended in 50 to 100 μL ultrapure glycerol (50% in autoclaved nanopure water) and transferred to a 96-well plate for storage at −80° C. Expression DWP culture was chilled on ice for 10 min, then induced by adding IPTG (1 mM final concentration) before incubating for 22 to 24 h at 23° C. with shaking at 9v00 rpm. The cells were harvested via centrifugation (2056×g, 4° C., 1 h) then stored at −80° C. for 20 min or overnight. Cell pellets were chemically lysed and the insoluble fraction was removed by centrifugation. The cell lysate was diluted into an activity buffer (50 mM HEPES pH 7.4, 0.05% Tween 20) and mixed with substrate for determining rates of proteolytic cleavage with a sensitive, robust fluorescence-polarization assay (Gilmore, M. A. et al. Anal. Biochem. 413, 36-42 (2011)) adapted to the composition, systems, and methods described herein.

### Example 6

### Exemplary Improved Substrate Specificity of Novel Modified Lc/a Proteases

Exemplary substrate specificity of the modified LC/A proteases are provided herein. Briefly, initial rates for each substrate is divided and compared to a standard (for example, an unevolved LC/A protease) to determine which modified LC/A protease demonstrate an improved specificity over the unevolved LC/A protease. The modified LC/A protease with improved specificity are then expressed and screened once more in triplicate to confirm results from the prior screen. The modified LC/A protease with highest specificity for the target substrate (referred to as a “selectant”) from each screen is then sequenced and taken forward for further mutagenesis and screening.

FIG. 4A, FIG. 4B, and FIG. 4C provide specificity data of exemplary modified LC/A protease variants. The slopes of initial cleavage rates of SNAP25 (SEQ ID NO: 25) (FIG. 4A) and SNAP23 (SEQ ID NO: 24) (FIG. 4B) by the modified LC/A proteases were divided and normalized to the corresponding cleavage rates of the protease variant of SEQ ID NO: 27 (used as the reference protease) to determine improvements in SNAP23 specificity (FIG. 4C). The data demonstrates that exemplary modified LC/A proteases represented in FIG. 4C, specifically, the modified LC/A protease of SEQ ID NO: 11 (which includes a S254L substitution) was over 100-fold and the modified LC/A protease of SEQ ID NO: 23 (which includes a S254M substitution) was over 40-fold more specific for SNAP23 over SNAP25 than the protease variant of SEQ ID NO: 27. The “mP” on y-axis represents fluorescence polarization in millipolarization units (mP) with time in seconds (s) on the x-axis.

### Substrate Specificity Determination of the Modified LC/a Proteases:

When evolving a modified LC/A protease for substrate specificity, cells from Example 5 were thawed at ambient temperature, then lysed at 23° C. with shaking at 500 rpm for 30 min in deep-well plate (DWP) Lysis Buffer: 100 μL per well of SoluLyse (Genlantis) plus benzonase nuclease (75 U/mL, NEB). The insoluble debris was removed via centrifugation (2056×g, 4° C., 1 h), then an aliquot (75 μL) of each lysate was transferred to a 96-well plate (Celltreat). Lysates were diluted 1:100, 1:200, 1:400, or 1:600 in assay buffer (50 mM HEPES, 0.05% v/v Tween, pH 7.4) or Intracellular Buffer (50 mM KH₂PO₄, pH 7.4) before screening.

Recombinantly expressed and purified SNAP25 substrate was diluted to 3 μM in Assay Buffer or Salt Buffer, then 100 μL substrate added to each well of a 96-well flat, black, non-binding surface microtiter plate (Corning) for screening via a robust, sensitive fluorescence polarization assay (Gilmore, M. A. et al. Anal. Biochem. 413, 36-42 (2011)) adapted to the composition, systems, and methods described herein. From the diluted lysate plate, 50 μL of the blank was added to its corresponding well in the black plate and used to optimize the gain and Z position of a Spark fluorescence polarization plate reader (Tecan). The sample was excited with polarized light at 380(20) nm and the polarized emission detected at 535(25) nm. For the remaining 95 wells, 50 μL from each well of the lysate dilution plate was added to the black plate containing substrate and the entire plate screened kinetically for 50 min to 14 h at 28±1° C. The assay steps were then repeated for 3 μM SNAP23 using the same lysate dilution plate.

The changes in polarization over time were visualized using Prism (GraphPad) and initial rates (Vo) were derived from fitting trendlines to the initial, linear portion of the raw data. The rates of negative controls (no enzyme) for each substrate were subtracted from the rates of each variant to account for nonenzymatic changes in polarization. The specificity indices were calculated via the ratio of SNAP23 and SNAP25 rates for each clone according to the equation below.

Specificity index=V₀²³/V₀²⁵

The modified LC/A variants with specificity indices at least 1.5 times higher than the protease variant of SEQ ID NO: 27 (used as the reference protease) or the most specific variant from the previous round of directed evolution were subject to further screening in triplicate. From the glycerol stocks, the modified LC/A protease and controls (the protease variant of SEQ ID NO: 27 and the modified LC/A protease from the previous round with the highest SNAP23 specificity) were streaked onto LBₖₐₙ plates for overnight incubation at 37° C. Three colonies from these streaks were used to inoculate three wells of a seed culture DWP and then three wells of an expression culture DWP were used for modified LC/A protease production, harvesting, lysis, and screening as described for the single well screen. The specificity index of each well was calculated first, then indices averaged together for the same modified LC/A protease. The modified LC/A proteases demonstrating consistent, improved specificity over the most SNAP23-specific modified LC/A protease from the previous round (referred to as “selectant”) were selected as starting points for the next round of mutagenesis and screening.

Table 7 demonstrates initial rates of SNAP cleavage of exemplary modified LC/A proteases for SNAP23 vs. SNAP25 substrates. Initial rates in columns with an asterisk (*) were not normalized for concentration of the protease and should be treated as estimations. As shown in Table 7, an exemplary modified LC/A protease with the following amino acid substitutions relative to the wild type protease (SEQ ID NO: 1): N26S, Q29R, N53R, E55V, E148Y, K166F, N240A, and S254L (SEQ ID NO: 12) demonstrates an increase in specificity for SNAP23 of more than 100-fold over the reference protease variant of SEQ ID NO: 27 in assay conditions for LC/A (assay buffer: 50 mM HEPES, 0.05% Tween, pH 7.4). The addition of S254L mutation was shown to increase SNAP23 specificity of the modified protease by over 100-fold over the reference protease.

As shown in Table 7, another exemplary modified LC/A protease with substitutions at positions N26S, Q29R, N53H, E55V, E148Y, K166F, N240A, and S254L (identified herein as SEQ ID NO: 13) demonstrates an increase in specificity for SNAP23 of at least about 1300-fold or more over the reference protease variant of SEQ ID NO: 27 in physiologically relevant salt conditions (e.g., 50 mM KH₂PO₄ pH 7.4). Such physiologically relevant salt concentrations significantly reduce SNAP23 cleavage with the protease variant of SEQ ID NO: 27. The N53H substitution was shown to increase SNAP23 specificity of the modified LC/A protease by at least 1300-fold or more over the protease variant of SEQ ID NO: 27. Further, it was determined that the D305 substitution can be reverted to the wild-type residue (G) without affecting specificity, and the E148Y and K166F substitutions provided for SNAP23 specificity.

Table 8 lists some kinetic parameters of SNAP cleavage of exemplary modified LC/A proteases for SNAP23 vs. SNAP25 substrates. As shown in Table 8, an exemplary modified LC/A protease with the following amino acid substitutions relative to the wild type protease (SEQ ID NO: 1): N26S, Q29R, N53H, E55V, E148Y, K166F, N240A, and S254L (SEQ ID NO: 13) demonstrates an increase in catalytic efficiency for SNAP23 over SNAP25 of 120-fold or more over the protease variant of SEQ ID NO: 27 in intracellular salt conditions supplemented with zinc (50 mM KH₂PO₄, 0.2 nM ZnCl₂ pH 7.4).

The lower rates of substrate cleavage with neuronal SNAP25 represent an improvement in safety for the evolved modified LC/A proteases as therapeutics capable of limiting off-target cleavage events. To summarize, the LC/A proteases described herein account for both decrease in native substrate activity (e.g., decreased SNAP25 cleavage) and increase in target substrate activity (e.g., increased SNAP23 cleavage) resulting in an evolution of overall substrate specificity, and thereby provide for a new class of non-cytotoxic therapeutic agent.

TABLE 7
Comparison of initial rates of SNAP cleavage of exemplary LC/A protease variant clones

| | | | S23/S25 rates | | |
|---|---|---|---|---|---|
| | Modified residues relative to | | normalized to | S25 rate vs. | S23 rate vs. |
| SEQ | wild-type BoNT/A light | S23/S25 | protease variant of | protease variant of | protease variant of |
| ID NO: | chain (SEQ ID NO: 1) | rates | SEQ ID NO: 27 | SEQ ID NO: 27* | SEQ ID NO: 27* |
| 27 | E148Y, K166F, S254A, G305D | 0.005 ± 0.001 | 1 | 1 | 1 |
| 5 | E148Y, K166F, N240S, S254A, | 0.007 | >1 | 0.57 | 0.40 |
| | G305D | | | | |
| 6 | E148Y, K166F, N240A, S254A, | 0.011 ± 0.001 | 1.3 | 0.64 | 0.84 |
| | G305D | | | | |
| 7 | N26S, E148Y, K166F, N240A, | 0.22 ± 0.01 | 6 | 0.74 | 3.41 |
| | S254A, G305D | | | | |
| 8 | N26S, E55V, E148Y, K166F, | 0.50 ± 0.1 | 13 | 0.42 | 4.65 |
| | N240A, S254A, G305D | | | | |
| 9 | N26S, Q29R, E55V, E148Y, | 0.73 ± 0.09 | 18 | 0.15 | 3.33 |
| | K166F, N240A, S254A, G305D | | | | |
| 10 | N26S, Q29R, N53R, E55V, E148Y, | 1.2 ± 0.6 | 30 | 0.04 | 0.69 |
| | K166F, N240A, S254A, G305D | | | | |
| 11 | N26S, Q29R, N53R, E55V, E148Y, | 4 ± 2 | 100 | 0.14 | 16.58 |
| | K166F, N240A, S254L, G305D | | | | |
| 12 | N26S, Q29R, N53R, E55V, E148Y, | 3.2 ± 0.2 | 80 | 0.04 | 12.2 |
| | K166F, N240A, S254L | | | | |
| 13 | N26S, Q29R, N53H, E55V, E148Y, | 2.4 ± 0.3 | 1300 | 0.02 | 34.04 |
| | K166F, N240A, S254L | | | | |

Initial rates in columns with an asterisk (*) were not normalized for concentration of the protease and should be treated as estimations. All rates were obtained with 2 μM substrate. The modified LC/A proteases were assayed in Intracellular Buffer (50 mM KH₂PO₄ pH 7.4).

TABLE 8
Comparison of kinetic parameters for SNAP cleavage of the protease
variant of SEQ ID NO: 13, the wild type BoNT/A light chain
(SEQ ID NO: 1) and the protease variant of SEQ ID NO: 27
under physiological salt conditions supplemented with zinc
(50 mM KH₂PO₄, 0.2 nM ZnCl₂ pH 7.4). Und. = undetectable.

| | | | | | | | | | S23/S25 |
|---|---|---|---|---|---|---|---|---|---|
| | SNAP23 | | | | SNAP25 | | | | Fold |
| SEQ | k_{cat} | Kₘ | Vₘₐₓ | k_{cat}/Kₘ | k_{cat} | Kₘ | Vₘₐₓ | k_{cat}/Kₘ | change/ |
| ID NO: | (s⁻¹) | (μM) | (mP*μM*s⁻¹) | (μM⁻¹*s⁻¹) | (s⁻¹) | (μM) | (mP*μM*s⁻¹) | (μM⁻¹*s⁻¹) | k_{cat}/Kₘ |
| 1 | und. | und. | und. | und. | 52 ± 2 | 3.4 ± 0.3 | 0.52 ± 0.02 | 15 ± 1 | — |
| 27 | 3.3 ± 0.4 | 18 ± 4 | 0.033 ± 0.004 | 0.18 ± 0.05 | 140 ± 9 | 0.7 ± 0.2 | 1.40 ± 0.09 | 200 ± 60 | 1 |
| 13 | 8.1 ± 0.3 | 7.4 ± 0.6 | 0.081 ± 0.003 | 1.1 ± 0.1 | 3.2 ± 0.1 | 0.32 ± 0.09 | 0.032 ± 0.001 | 10 ± 3 | 120 |

### Example 7

### Exemplary Specificity Assay of Novel Modified Lc/a Proteases Generated with Error-Prone PCR Technique

LC/A protease gene variants were generated with error-prone PCR (epPCR) technique with the GeneMorph II kit (Agilent) for developing modified LC/A proteases with improved SNAP23 specificity. The resulting LC/A protease gene variants were cloned into a pET29b(+) vector and the presence of full-length LC/A protease gene variants and random mutagenesis were confirmed with Sanger sequencing.

Protein overexpression and purification of modified LC/A proteases for specificity assay: The resulting LC/A protease gene variants were transformed into BL21 (DE3) Star E. coli and plated on an LB/agar plate with kanamycin for overnight growth at 37° C. Single colonies were picked and used for inoculating a 96 deep well plate (DWP) seed cultures. The seed culture included 300 μL of LB with 40 μg/mL kanamycin (LB/kan) per well, grown at 37° C. with shaking at 225 rpm for 18 h. In addition to the LC/A protease gene variants, the protease variant of SEQ ID NO: 27 (used as the reference protease) and an uninoculated well were added as controls (positive and blank controls, respectively). Next, 20 μL of the seed culture was used to inoculate a 96 DWP expression culture (630 μL of LB/kan per well) and grown for 3 h at 37° C. with shaking at 225 rpm. The expression plate was cooled on ice for 10 min, then induced by adding 6.5 μL of 100 mM IPTG and incubated overnight for 22 to 24 h at 23° C. with shaking at 900 rpm. Cells were harvested via centrifugation (3000 rpm, 4° C., 1 h). Harvested cells were lysed by freezing at −80° C. for 20 min, thawed at room temperature, and then incubated at 23° C., 500 rpm for 30 min with 11 mL SoluLyse (Genlantis) and 3.3 μL Benzonase nuclease (NEB)/100 μL per well. Insoluble debris was separated from cytosol via centrifugation (3000 rpm, 4° C., 1 h). Lysates were transferred to a 96-well plate (Celltreat) and diluted to 1:200 in assay buffer (50 mM HEPES pH 7.4 0.05% v/v Tween).

Specificity assay: SNAP DARET assay substrates (SNAP25 and SNAP23) were diluted to 2 μM in assay buffer. 100 μL of SNAP25 was added to each well of a 96-well flat, black, non-binding surface plate (Corning), adding 50 μL of the blank well from the dilution plate to the black plate for optimizing the gain and Z position of the fluorescence polarization plate reader. Lastly, 50 μL of the remaining 95 wells were added from the dilution to the black plate for the assay. The assay steps were repeated for SNAP23 with the same dilution plate. The substrates were excited at 380(20) nm and emission was monitored at 535(25) nm. Specificity index was calculated by dividing initial cleavage rate of SNAP23 by initial rate of SNAP25 for any given modified LC/A protease as described in Example 6.

FIGS. 5A-5D provide specificity data of exemplary modified LC/A proteases generated with the error-prone PCR (epPCR) technique. Initial linear cleavage rates derived from the cleavage of SNAP25 (SEQ ID NO: 25) (shown in FIG. 5A) and SNAP23 (SEQ ID NO: 24) (shown in FIG. 5B) by the exemplary modified LC/A proteases were divided and normalized to the corresponding cleavage rates of the protease variant of SEQ ID NO: 27 to identify modified LC/A proteases with similar or improved SNAP23 specificity (shown in FIG. 5C). Selected modified LC/A proteases were screened again in triplicate to reveal that a modified LC/A protease with an N240S substitution (SEQ ID NO: 5) and a modified LC/A protease with the combination of an E201D and a D203V substitution (SEQ ID NO: 14) demonstrated improved SNAP23 specificity of 1.2- and 3.5-fold, respectively, over the protease variant of SEQ ID NO: 27 (shown in FIG. 5D). Abbreviations used in FIGS. 5A-5D: mP denotes Fluorescent Polarization; N240S denotes the modified LC/A protease of SEQ ID NO: 5; E201D/D203V denotes the modified LC/A protease of SEQ ID NO: 14; qmLC/A denotes the protease variant of SEQ ID NO: 27.

### Example 8

### Exemplary Specificity Assay of Novel Modified Lc/a Proteases Generated with DNA Shuffling

LC/A protease gene variants were generated with DNA shuffling technique for developing modified LC/A proteases with improved SNAP23 specificity. The resulting LC/A protease gene variants were grown and screened as described in Example 7. The process resulted in silent mutations on the E201D/D203V backbone (the modified LC/A protease of SEQ ID NO: 14) as shown in FIG. 6C.

FIGS. 6A-6C provide specificity data of exemplary modified LC/A proteases generated with DNA shuffling technique. Initial linear cleavage rates derived from cleavage of SNAP25 (SEQ ID NO: 25) (shown in FIG. 6A) and SNAP23 (SEQ ID NO: 24) (shown in FIG. 6B) by the exemplary modified LC/A proteases were divided and normalized to the corresponding cleavage rates of the protease variant of SEQ ID NO: 27 (used as the reference protease) to determine which modified LC/A proteases have similar or improved SNAP23 specificity over the modified LC/A protease with N240S substitution (SEQ ID NO: 5) and LC/A protease with the combination of E201D and D203V substitutions (data shown in FIG. 6C). Abbreviations used in FIGS. 6A-6C: mP denotes Fluorescent Polarization; N240S denotes the modified LC/A protease of SEQ ID NO: 5; E201D/D203V denotes the modified LC/A protease of SEQ ID NO: 14; qmLC/A denotes the protease variant of SEQ ID NO: 27.

### Example 9

### Modulating Specificity of Bont Proteases

Methods for modulating the substrate specificity of the BoNT proteases are provided. Purification of an exemplary modified LC/A protease (SEQ ID NO: 13) from cell lysate unexpectedly yielded a protease that cleaves SNAP25 approximately five (5) times faster than SNAP23 as assessed by a fluorescence-polarization assay (FIG. 8A). Purification of the modified LC/A protease (SEQ ID NO: 13) using a zinc-charged metal affinity chromatography resin instead of a nickel-charged resin in low-salt conditions (50 mM HEPES, 100 mM NaCl pH 8.0, with 0, 20, or 250 mM imidazole) improved the protease's SNAP23 cleavage rate approximately by 3.5 folds (FIG. 8B).

To examine the influence of zinc (Zn²⁺) on the LC/A protease specificity, the modified LC/A protease (SEQ ID NO: 13) was dialyzed into either the assay buffer (50 mM HEPES, pH 7.4) or a zinc buffer (50 mM HEPES, 0.2 mM ZnCl₂, pH 7.4). FIGS. 8C-8E demonstrate that dialysis of the modified LC/A protease (SEQ ID NO: 13) purified in low-salt conditions into zinc buffer (50 mM HEPES, 0.2 mM ZnCl₂, pH 7.4) yielded a LC/A protease that cleaves SNAP23 approximately four (4) times faster than SNAP25. Individual initial cleavage rates for SNAP23 and SNAP25 for the purified LC/A protease (SEQ ID NO: 13) dialyzed into the assay buffer or the zinc buffer are shown in FIG. 8C and FIG. 8D, respectively. Dividing the initial rates yielded the specificity index depicted in FIG. 8E. For each assay, the final concentration of the protease was 50 nM and the final concentration of each substrate was 2 μM. For data shown in FIGS. 8A-8E, the final concentration of the enzyme was 50 nM and the final concentration of each substrate was 2 μM. The diluent used to prepare the protease and substrate for each assay was 50 mM KH₂PO₄, 0.2 mM ZnCl₂, pH 7.4.

FIGS. 8A-8E provide exemplary data demonstrating that the modified LC/A protease (SEQ ID NO: 13) dialyzed in zinc buffer cleaved a SNAP23 substrate with a higher rate than a SNAP25 substrate. As shown in FIGS. 8A-8C, the modified LC/A protease (SEQ ID NO: 13) exhibits strong dependence on the presence of Zn²⁺ for its substrate specificity, but not its activity. In the absence of additional Zn²⁺ (e.g., in assay buffer (50 mM HEPES, pH 7.4) or intracellular buffer (50 mM KH₂PO₄, pH 7.4)), the modified LC/A protease (SEQ ID NO: 13) remains proteolytic, but has a higher rate of cleavage for SNAP25 than SNAP23. The addition of 0.2 mM Zn²⁺ to either assay or intracellular buffer reverses this specificity, and the modified LC/A protease's specificity for SNAP23 is restored. Thus, the zinc-mediated modification provides a novel control element and/or a co-factor for modulating substrate specificities of the BoNT proteases.

Further, this Zn²⁺-dependent substrate specificity suggests new possibilities for modulating the BoNT protease activity. Notably, the exemplary modified LC/A protease (SEQ ID NO: 13) has relatively weak affinity for Zn²⁺, as compared to the affinity for Zn²⁺ of the active site in the protease. The active site however remains intact as evidenced by proteolysis of SNAP23 by the modified LC/A protease (SEQ ID NO: 13) even without addition of Zn²⁺ to the assay or intracellular buffer. It is understood that cells with low concentrations of free Zn²⁺, for example, would be excluded from such zinc-mediated activity. This possibility illustrates a new mechanism for achieving cell specificity, based upon intracellular Zn²⁺ concentration. Alternatively, additional protein engineering, as described herein, could remove the Zn²⁺ sensitivity for the exemplary modified LC/A protease (SEQ ID NO: 13).

Modifying the proteases' substrate specificity with zinc revealed a new series of residues in the proteases that exert control over the substrate specificity of the protease. In particular, altering substrate specificity in the proteases described herein was found to involve modification of seven residues occupying two loops (loop one spanning residues 26-29 and loop two spanning 52-56 of the LC/A protease of SEQ ID NO: 13), which are referred to herein as “substrate control” loops.

Furthermore, the newly introduced Zn²⁺ binding site illustrates a novel method of ligand-based additional control over the protease's substrate specificity function. It is understood that such ligand-based control can be utilized to increase the new binding site's affinity for a zinc ion and also to select for other suitable ligands. Small molecules, for example, could be used in place of Zn²⁺ to complete and/or facilitate formation of the LC/A-SNAP23 complex necessary for improved protease activity. Other bivalent metal ions could also be used for binding to those sites to offer a new level of enzymatic control for the proteases. One could supplement a formulation with sufficient zinc to insure substrate specificity for therapeutic use.

### Example 10

### Exemplary Modified Lc/E Proteases and Substrate Co-Evolved to Cleave Snap29

Modified LC/E proteases that cleave a non-canonical substrate, e.g., SNAP29, are provided herein. For applying the LC/A protease library platform to LC/E and SNAP29, protease libraries using a substrate coevolution technique to engineer target cleavage (Chen, Z. & Zhao, H. J. Mol. Biol. 348, 1273-1282 (2005)) was generated. Briefly, first protease variants were screened against a native, target chimeric protein, then over successive rounds of evolution, native amino acids (e.g., SNAP25) are swapped for target ones (e.g., SNAP29) until the substrate is 100% target. This strategy is effective when the starting protease has no activity against the target substrate, as with LC/E protease and SNAP29. Using this technique, a SNAP25/29 chimeric substrate was created that is susceptible to LC/E protease cleavage. The chimeric substrate was screened against LC/E protease library variants (FIGS. 7A and 7B). The best modified LC/E proteases are further evolved for improved SNAP29 cleavage. FIGS. 7A-7B demonstrate modified LC/E proteases (generated using LC/E protease of SEQ ID NO: 28) that cleave SNAP29 (SEQ ID NO: 4) and SNAP25/29 chimeric substrate (SEQ ID NO: 29) on the LC/A protease library platform via coevolution. FIG. 7A demonstrates that an exemplary SNAP25/29 (SEQ ID NO: 29) chimeric substrate (19% SNAP25, 81% SNAP29) was cleaved by wild-type LC/E in a fluorescence-polarization assay. Trypsin was used as a positive control in the assay. FIG. 7B demonstrates that at least five exemplary modified LC/E proteases screened against both the SNAP25/29 chimeric substrate and SNAP25 showed potentially improved specificity for the chimeric substrate. Abbreviations used in FIG. 7A and FIG. 7B: S29/25 chimera and S29/25 denote SNAP25/29 chimeric substrate (SEQ ID NO: 29); S25 denotes SNAP25 substrate (SEQ ID NO: 25); S29 denotes SNAP29 substrate (SEQ ID NO: 4). The modified LC/E provide a new class of non-cytotoxic therapeutic agents.

### Example 11

### Modified Bont/a Proteases and Specificity and Functionality Assays Thereof

Botulinum neurotoxin proteins are prepared that contain a modified L-chain protease having one or more of the amino acid modifications set forth in Tables 3 and 4. In brief, full length BoNT/A proteases are engineered with oligonucleotides encoding LC/A protease variants having one or more of the exemplary amino acid substitutions as shown in Tables 3 and 4. Resulting modified BoNT/A proteases are recombinantly expressed in E. coli and purified. Substrate specificity is evaluated as described in above. For further evaluation, modified BoNT/A proteases are independently assayed for SNAP25 and SNAP23 cleavage in PC12 cell-based potency assay. PC-12 cells express both SNAP25 and SNAP23 and their cleavage in cells is an indirect measure of the four steps of BoNT functional activity: 1) Cell binding, 2) Internalization, 3) Translocation and 4) Proteolysis. Results demonstrate increased potency (enhanced SNAP23 cleavage) for modified BoNT/A proteases compared to the native BoNT/A in PC12 cells. In contrast, SNAP25 cleavage is reduced in PC12 cells treated with modified BoNT/A proteases. This demonstrates that the modified BoNT/A proteases specifically cleave SNAP23 and concomitantly have a reduced ability to cleave SNAP25.

A modified BoNT/A protease (“omBoNT/A”) comprising the modified LC/A protease of SEQ ID NO: 13 (referred in this experiment as “omLC/A”) was used as the exemplary modified BoNT/A protease in the following experiments:

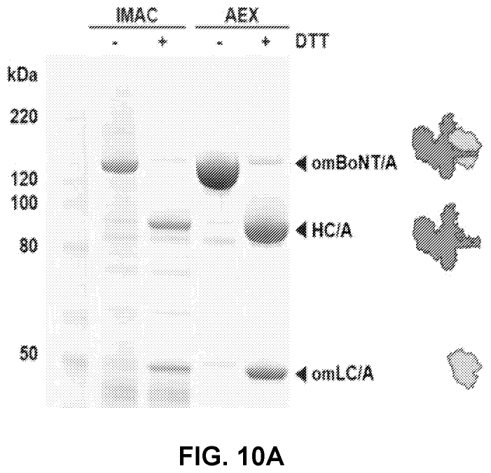

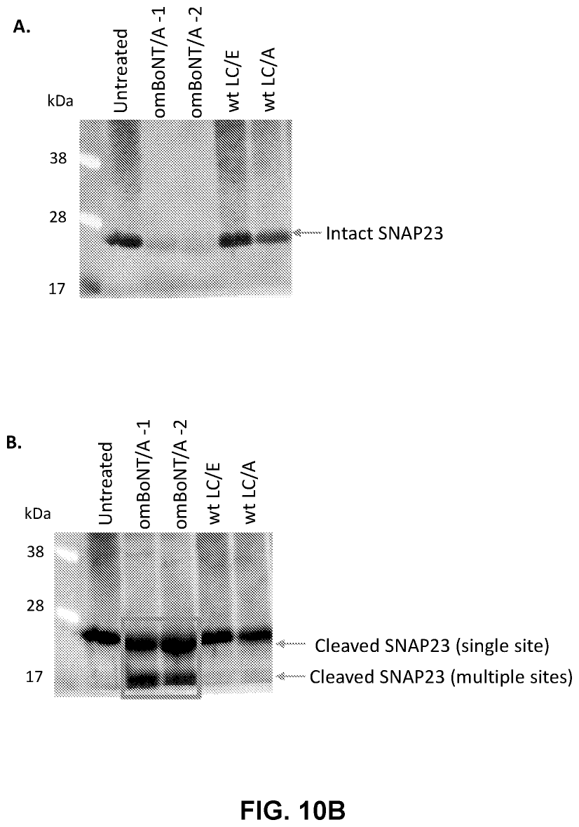

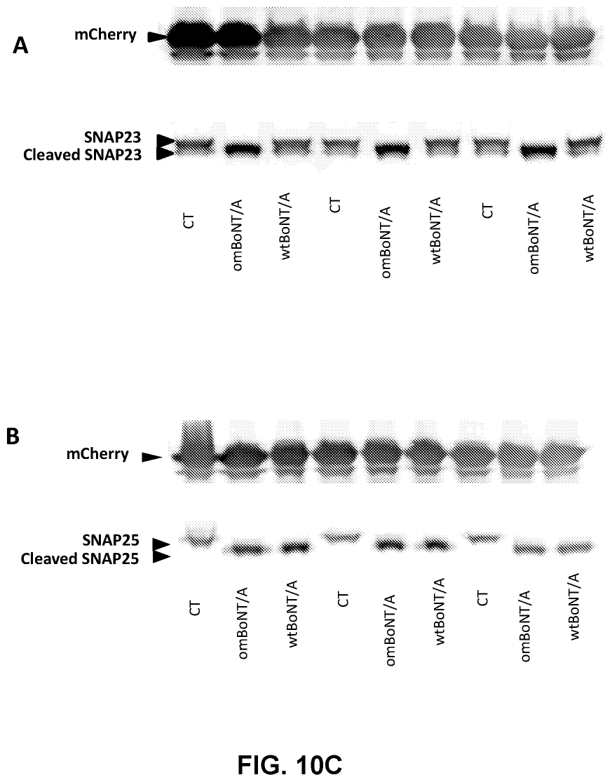

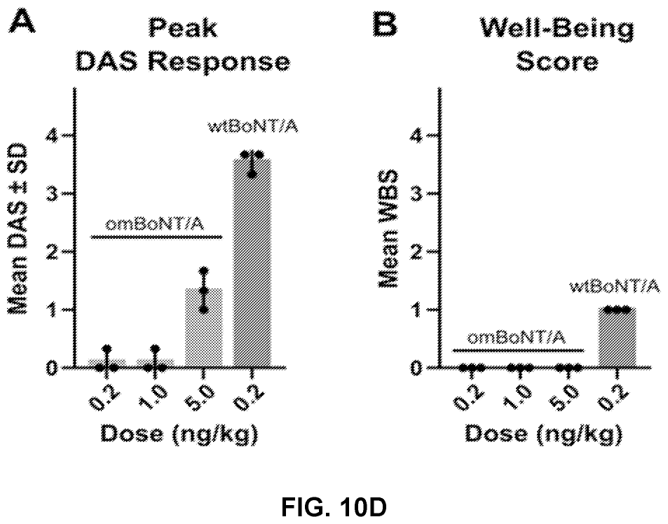

A. Construction, Expression, and Purification of omBoNT/A: The gene encoding omBoNT/A was constructed through overlap extension PCR using the gene encoding omLC/A (SEQ ID NO: 13, having the following amino acid substitutions: N26S/Q29R/N53H/E55V/E148Y/K166FN240A/S254L) as the template. The resulting PCR products were DpnI-digested, column purified and concentrated, then ligated and subcloned via ligation independent cloning (LIC) into a pET-29b(+) vector featuring full-length wild-type BoNT/A (DNA001462). Sanger sequencing confirmed construction of omBoNT/A-pET-29b(+) (DNA002109). omBoNT/A was expressed in 1.5 L of Terrific Broth supplemented with 1% glucose (w/v) until the culture reached an OD of 0.6. The culture was then induced with 0.2 mM IPTG and incubated at 16° C., 265 rpm for 20 hours. After cell harvest and chemical lysis (FastBreak™, Promega), omBoNT/A was purified by immobilized metal affinity chromatography (IMAC) on MagneHis™ resin followed by anion exchange chromatography on a Hitrap® Q HP column (Cytiva). omBoNT/A was eluted with a linear gradient of NaCl ranging from 80 mM to 1 M NaCl. Pooled anion exchanged fractions were exchanged into 50 mM Tris pH 8.0, 120 mM NaCl, 0.1 mM ZnCl₂ and 5% PEG (v/v) 400 (#8074850050, Sigma). Fractions were analyzed for nicking in the presence or absence of 100 mM DTT by SDS PAGE, staining with Spyro™ Ruby (Thermo). FIG. 10A shows the image of the SDS PAGE with omBoNT/A, HC/A, and omLC/A schematically represented on the right side.

B. In vitro Cleavage of Human rSNAP23 with omBoNT/A: Human SNAP23 in vitro cleavage was evaluated by incubating 30 μg of full-length human recombinant SNAP23 protein (23 kDa) with 400 nM of either wild type LC/A, wild type LC/E, or reduced omBoNT/A at 37° C. for 1 hour in PBS, pH 7. omBoNT/A was reduced by incubation with 2 mM TCEP (Tris-(2-carboxyethly) phosphine, hydrochloride) at 37° C. for 4 hours. The reaction was stopped by addition of NuPAGE™ LDS Sample Buffer and the amount of SNAP23 was assessed by Anti-SNAP23 Western blot analysis. Reaction samples (about 24 μg) were separated by SDS-PAGE (12% acrylamide) and transferred to nitrocellulose membrane, 0.45 μm pore size in 1× Transfer buffer containing 20% v/v methanol. Membranes were blocked in 2% ECL Prime™ Blocking Agent in TBST (Tris-Buffered Saline) with 0.1% Tween-20 for 1 hour at room temperature. Intact and cleaved SNAP-23 protein was detected with either anti-SNAP23 polyclonal antibody against C-terminus or N-terminus of SNAP23 diluted 1:1000 in 2% blocking buffer. Blots were incubated overnight with primary antibodies at 4° C. with gentle agitation. Blots were washed in TBST and the bound antibody was detected after 1-hour incubation at room temperature with HRP-Goat Anti-Rabbit IgG (H-L) diluted 1:4000 in 2% blocking buffer. After final washes in TBST, the membranes were reacted with Pierce™ ECL Plus Western Blotting Substrate and scanned using the Typhoon 9410 Imager.

As shown in FIG. 10B, treatment with two independent preparations of omBoNT/A toxin (1 and 2) resulted in in vitro cleavage of recombinant human SNAP23 (23 kDa) as visualized by disappearance of intact hSNAP23 band with a C-terminal anti-SNAP23 antibody (FIG. 10B, panel A) and detection of cleaved SNAP23 (two bands) with a N-terminal anti-SNAP23 antibody (FIG. 10B, panel B). The untreated, wtLC/A, and wtLC/E lanes provided negative controls.

C. SNAP23 Cleavage in Human Neuroblastoma SiMa Cells: Human neuroblastoma SiMa H1 cells were differentiated for 3 days in differentiation media (Neurobasal Media, 1×GlutaMAX™, 1×B27 supplemented with ganglioside GT1b trisodium at 25 μg/mL). Cells were then infected for 24 hours with an Adenovirus Human Type 5 (dE1/E3) co-expressing m-Cherry and human SNAP23 under two independent CMV promoters in differentiation media containing GT1b. In 1 mL, 75×10⁶ PFU/well was used for infection. 24 hours after infection, cells were treated or not with either 50 nM of omBoNT/A or native wtBoNT/A (Metabiologics). After 48 hours of incubation, cells were lysed in 120 μL of ice-cold lysis buffer containing (20 mM Tris, pH 7.5, 150 mM NaCl, 1 mM EDTA, 1 mM EGTA, 1% Triton X-100 and 1X of Halt™ Protease and Phosphatase Inhibitor Cocktail). After migration on 12% SDS-PAGE, proteins were transferred onto nitrocellulose membranes and saturated for 1 hour at room temperature with Intercept® Blocking Buffer. Blots were then incubated with mouse anti-mCherry antibody and either anti-SNAP23 or anti-SNAP25 rabbit polyclonal antibody to the N-terminus of SNAP23 or SNAP25, respectively, in Intercept® Antibody Diluent overnight at 4° C. After washing in TBS-Tween 0.05%, membranes were incubated with secondary antibodies, goat anti-mouse IRDye 680RD and goat anti-rabbit IRDye 800CW, for 1 hour at room temperature. Membranes were then scanned with the Odyssey® CLx imaging system.

FIG. 10C shows that treatment of SiMa cells, expressing both SNAP23 and SNAP25, with 50 nM omBoNT/A resulted in cleavage of SNAP23 (lower band in FIG. 10C, panel A). This effect was specific to omBoNT/A as wtBoNT/A (50 nM) did not cleave SNAP23 (FIG. 10C, panel A). At the same concentration (50 nM), both omBoNT/A and wtBoNT/A cleaved SNAP25 (FIG. 10C, panel B).

D. Mouse Digital Abduction (DAS) Assay: All procedures were approved by AACUC (approved protocol #225-100051-2019). The DAS assay was performed using methods in the art. Female CD-1 mice (Charles River), with an average weight of 30.2 g and age range of 6-10 weeks old, were used. The omBoNT/A and native wt BoNT/A neurotoxin (Metabiologics Inc.) were diluted in 0.5% human serum albumin in 0.9% saline (Fresenius Kabi, 918620). For the assay, 0.005 mL of each diluted toxin was injected in the right gastrocnemius muscle. Three mice per dose (n=3) were tested in triplicates (N=3). The DAS score, the Well-Being score, and weight were recorded daily for 4 days. The results were plotted using Prism (GraphPad). Briefly, in the DAS assay, mice were suspended briefly by the tail to elicit a characteristic startle response in which the mouse extends its hind limbs and abducts its hind digits. Following toxin injection, the varying degrees of digit abduction were scored on a five-point scale (0 to 4, where 0=normal, no digit abduction, and 4=maximal reduction in digit abduction and leg extension). The Well-Being of each subject was scored on a 4-point system (0=activity level normal; 1=slightly diminished activity level and/or slight weight loss (5-10%); 2=moderately diminished activity level and moderate weight loss (10-15%); 3=severely diminished activity level, little to no reaction to outside stimuli, inability to ambulate, agonal or labored respiration).

FIG. 10D shows that, in vivo, omBoNT/A provided at least 25-fold reduced muscle paralysis associated with SNAP25 cleavage compared to wtBoNT/A. The residual paralysis observed demonstrates successful cellular delivery of active omLC/A into motor nerve terminals. Furthermore, no systemic toxicity effects were observed for mice treated with 5 ng/kg omBoNT/A. Together with the in vitro findings, these data indicate that the amino acids substitutions changing selectivity towards SNAP23 do not significantly alter the delivery of the omBoNT/A to the presynaptic compartment of neurons compared to wtBoNT/A.

While a number of exemplary aspects and embodiments have been discussed above, those of skill in the art will recognize modifications, permutations, additions and sub-combinations thereof. It is therefore intended that the following appended claims and claims hereafter introduced are interpreted to include all such modifications, permutations, additions, and sub-combinations as are within their true spirit and scope.

1. A botulinum neurotoxin protein, comprising: an amino acid sequence with at least about 90% sequence identity to SEQ ID NO: 1 and two or more amino acid substitutions selected from the group consisting of:
N26X1, wherein X1 is S, T, M, or C;
A27X2, wherein X2 is L, R, I, V, M, K, or Q;
Q29X3, wherein X3 is R, S, K, M, I, or T;
N53X4, wherein X4 is H, R, Q, K, M, or I;
E55X5, wherein X5 is I, N, V, L, M, Q, H, or D;
E56X6, wherein X6 is I, L, V, or M;
Q162X7, wherein X7 is R, K, M, or I;
E201X8, wherein X8 is D, N, or Q;
D203X9, wherein X9 is V, I, L, or M;
N240X10, wherein X10 is A, S, G, C, T, or M;
5254X11, wherein X11 is A, L, M, I, V, G, or C;
K364X12, wherein X12 is R, Q, M, or I; and
Y387X13, wherein X13 is N, Q, H, E, or D,
or a fragment thereof.

2. The botulinum neurotoxin protein or fragment thereof according to claim 1,
(a) further comprising one or more additional amino acid substitutions selected from the group consisting of:
E148X14, wherein X14 is Y, W, F, or H;
K166X15, wherein X15 is F, M, L, Y, W, or H; and
G305X16, wherein X16 is G, D, E, N, or Q;
(b) wherein the two or more amino acid substitutions are selected from the group consisting of:
N26X1, wherein X1 is S;
A27X2, wherein X2 is L or R;
Q29X3, wherein X3 is R or S;
N53X4, wherein X4 is H or R;
E55X5, wherein X5 is I, N, or V;
E56X6, wherein X6 is I;
Q162X7, wherein X7 is R;
E201X8, wherein X8 is D;
D203X9, wherein X9 is V;
N240X10, wherein X10 is A or S;
5254X11, wherein X11 is A, L, or M;
K364X12, wherein X12 is R; and
Y387X13, wherein X13 is N; and/or
(c) wherein the one or more additional acid substitutions are selected from the group consisting of:
E148X14, wherein X14 is Y;
K166X15, wherein X15 is F; and
G305X16, wherein X16 is G or D.

3-4. (canceled)

5. The botulinum neurotoxin protein or fragment thereof according to claim 2,
wherein X14 is Y;
wherein X15 is F; and
wherein X16 is G or D,
with the proviso that the amino acid sequence is not SEQ ID NO: 27.

6. The botulinum neurotoxin protein or fragment thereof according to claim 1, wherein
(a) the amino acid sequence comprises the S254X11 amino acid substitution wherein X11 is L or M;
(b) the amino acid sequence comprises an N53H amino acid substitution;
(c) the amino acid sequence comprises E201D and D203V amino acid substitutions; and/or
(d) the amino acid sequence comprises one or more of the following amino acid substitutions: N26S, Q29R, E55V, E148Y, K166F, N240A, G305D.

7-9. (canceled)

10. The botulinum neurotoxin protein or fragment thereof according to claim 1, said amino acid sequence comprises:
(a) at least about 90% sequence identity to SEQ ID NO: 1 and wherein the amino acid sequence has at least two amino acid positions with an amino acid modification set forth in Table 1 and at least one amino acid position with one of the amino acid modifications set forth in Table 3;
(b) at least about 90% sequence identity to SEQ ID NO: 1 and wherein the amino acid sequence has (i) one set of amino acid modifications set forth in Table 2 and (ii) one or more amino acid positions with an amino acid modification set forth in Table 3;
(c) at least about 90% sequence identity to SEQ ID NO: 1 and wherein the amino acid sequence has one or more amino acid positions with one or more of the amino acid substitutions set forth in Table 3;
(d) at least about 90% sequence identity to SEQ ID NO: 1 and wherein the amino acid sequence has (i) at least two amino acid positions modified by an amino acid modification set forth in Table 1 and (ii) one or more of the amino acid modifications set forth in Table 4 or one set of amino acid modifications set forth in Table 4;
(e) at least about 90% sequence identity to SEQ ID NO: 1 and wherein the amino acid sequence has (i) at least one set of amino acid modifications set forth in Table 2 and (ii) one or more of the amino acid modifications set forth in Table 4 or one set of amino acid modifications set forth in Table 4;
(f) at least about 90% sequence identity to SEQ ID NO: 1 and wherein the amino acid sequence has (i) at least 2 of the following amino acid substitutions: E148Y, K166F, S254A, G305D, and (ii) one or more of the amino acid substitutions set forth in Table 3;
(g) at least about 90% sequence identity to SEQ ID NO: 1 and wherein the amino acid sequence has (i) at least 2 of the following amino acid substitutions: E148Y, K166F, S254A, G305D, and (ii) one or more of the amino acid substitutions set forth in Table 4 or one set of amino acid substitutions set forth in Table 4;
(h) at least about 90% sequence identity to SEQ ID NO: 1 and wherein the amino acid sequence has one set of amino acid substitutions set forth in Table 5;
(i) at least about 90% sequence identity to SEQ ID NO: 1 and wherein the amino acid sequence has one set of amino acid substitutions set forth in Table 6; or
(j) at least about 90% sequence identity to SEQ ID NO: 1 and wherein the amino acid sequence has one of the following amino acid substitutions: (i) N53H, (ii) E148Y, (iii) K166F, (iv) E148Y and K166F, (v) S254L, or (vi) S254M.

11-19. (canceled)

20. The botulinum neurotoxin protein or fragment thereof according to claim 1, wherein the protein or fragment cleaves human SNAP23.

21. The botulinum neurotoxin protein or fragment thereof according to claim 20, wherein
(a) the protein or fragment is at least about 1.5-fold more specific for SNAP23 than for SNAP25;
(b) the protein or fragment is at least about 5-fold more specific for SNAP23 than for SNAP25;
(c) the protein or fragment is at least about 10-fold more specific for SNAP23 than for SNAP25;
(d) the protein or fragment is at least about 10-fold more specific for cleaving SNAP23 than a botulinum neurotoxin protein with an amino acid sequence having these four modifications: E148Y, K166F, S254A, G305D;
(e) the protein or fragment is at least about 20-fold more specific for cleaving SNAP23 than a botulinum neurotoxin protein with an amino acid sequence having these four modifications: E148Y, K166F, S254A, G305D;
(f) the protein or fragment is at least about 40-fold more specific for cleaving SNAP23 than a botulinum neurotoxin protein with an amino acid sequence having these four modifications: E148Y, K166F, S254A, G305D;
(g) the protein or fragment is at least about 40-fold more specific for cleaving SNAP23 than a botulinum neurotoxin protein with an amino acid sequence having these four modifications: E148Y, K166F, S254A, G305D, and the protein or fragment thereof comprises the amino acid substitution of S254M; or
(h) the protein or fragment is at least about 100-fold more specific for cleaving SNAP23 than a botulinum neurotoxin protein with an amino acid sequence having these four modifications: E148Y, K166F, S254A, G305D.

22-28. (canceled)

29. The botulinum neurotoxin protein or fragment thereof according to claim 20, wherein the protein or fragment is at least about 100 fold more specific for cleaving SNAP23 than a botulinum neurotoxin protein with an amino acid sequence having these four modifications: E148Y, K166F, S254A, G305D, and the protein or fragment thereof comprises the amino acid substitution of S254L or the protein or fragment thereof comprises the amino acid substitution of N53H.

30. (canceled)

31. The botulinum neurotoxin protein or fragment thereof according to claim 20, wherein the protein or fragment thereof comprises the amino acid substitution of N53H; and
(a) the protein or fragment is at least about 1300-fold more specific for cleaving SNAP23 than a botulinum neurotoxin protein with an amino acid sequence having these four modifications: E148Y, K166F, S254A, G305D, and wherein the at least about 1300-fold more SNAP23 cleavage specificity is obtained under physiological salt conditions; or
(b) the protein or fragment is at least about 120-fold more specific for cleaving SNAP23 than a botulinum neurotoxin protein with an amino acid sequence having these four modifications: E148Y, K166F, S254A, G305D, and wherein the at least about 120-fold more SNAP23 cleavage specificity is obtained under physiological salt conditions supplemented with zinc.

32-34. (canceled)

35. The botulinum neurotoxin protein or fragment thereof according to claim 1,
and further comprising a heavy chain protein from a botulinum neurotoxin or fragment thereof.

36. (canceled)

37. The botulinum neurotoxin protein or fragment thereof according to claim 1, said amino acid sequence comprising SEQ ID NO: 28.

38-40. (canceled)

41. The botulinum neurotoxin protein or fragment thereof according to claim 1, wherein the protein or fragment thereof has improved specificity for a non-canonical substrate relative to its canonical substrate, and wherein.
(a) the canonical substrate is SNAP25 and the non-canonical substrate is SNAP23, SNAP29, or a SNAP25/29 chimeric substrate;
(b) the canonical SNAP25 substrate comprises the amino acid sequence of SEQ ID NO: 25;
(c) the non-canonical SNAP23 substrate comprises the amino acid sequence of SEQ ID NO: 24;
(d) the non-canonical SNAP29 substrate comprises the amino acid sequence of SEQ ID NO: 4; or
(e) the non-canonical SNAP25/29 chimeric substrate comprises the amino acid sequence of SEQ ID NO: 29.

42-47. (canceled)

48. A nucleic acid encoding the botulinum neurotoxin or fragment thereof of according to claim 1.

49. A plasmid or vector comprising the nucleic acid of claim 48.

50. (canceled)

51. A host cell comprising the vector of claim 49.

52. An expression system comprising the host cell of claim 51, wherein the expression system is selected from the group consisting of bacteria, yeast, baculovirus in insect cell, cell-free expression, mammalian cell lines, animals, and phage.

53. (canceled)

54. A method of generating the botulinum neurotoxin protein or fragment thereof according to claim 1, comprising culturing a host cell comprising a nucleic acid encoding the botulinum neurotoxin or fragment thereof under conditions sufficient for the expression of the botulinum neurotoxin protein or fragment thereof, and obtaining the botulinum neurotoxin protein or fragment thereof from the culture.

55-58. (canceled)

59. A method for engineering a protease domain of a botulinum neurotoxin protein, or fragment thereof, according to claim 1, the method comprising:
(i) identifying sites in a protease domain of a botulinum neurotoxin or fragment thereof involved in substrate binding and/or cleavage;
(ii) constructing a library of protease domain gene mutants of botulinum neurotoxin or fragment thereof for the identified sites;
(iii) transforming each gene mutant in the library into an expression system;
(iv) expressing protein from clonal populations of each expression system;
(v) testing the expressed protein for binding to or cleavage of a non-canonical substrate to identify expressed proteins with improved substrate binding or cleavage;
(vi) sequencing protein identified to have improved substrate cleavage; and
(vii) repeating steps (ii)-(vi) using the sequence identified in (vi).


SEQUENCE LISTING (32 sequences)

SEQ ID NO: 1 - protein, 448 aa, Artificial Sequence
  Wild-type botulinum neurotoxin serotype A (BoNT/A) light chain (amino acid residues 1 - 448 of UniProt P0DPI1)
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNK

SEQ ID NO: 2 - protein, 211 aa, Artificial Sequence
  Human SNAP23 of Uniprot O00161
MDNLSSEEIQQRAHQITDESLESTRRILGLAIESQDAGIKTITMLDEQKEQLNRIEEGLDQINKDMRETEKTLTELNKCCGLCVCPCNRTKNFESGKAYKTTWGDGGENSPCNVVSKQPGPVTNGQLQQPTTGAASGGYIKRITNDAREDEMEENLTQVGSILGNLKDMALNIGNEIDAQNPQIKRITDKADTNRDRIDIANARAKKLIDS

SEQ ID NO: 3 - protein, 206 aa, Artificial Sequence
  Human and rodent SNAP25 of UniProt P60880
MAEDADMRNELEEMQRRADQLADESLESTRRMLQLVEESKDAGIRTLVMLDEQGEQLERIEEGMDQINKDMKEAEKNLTDLGKFCGLCVCPCNKLKSSDAYKKAWGNNQDGVVASQPARVVDEREQMAISGGFIRRVTNDARENEMDENLEQVSGIIGNLRHMALDMGNEIDTQNRQIDRIMEKADSNKTRIDEANQRATKMLGSG

SEQ ID NO: 4 - protein, 258 aa, Artificial Sequence
  Human SNAP29
MSAYPKSYNPFDDDGEDEGARPAPWRDARDLPDGPDAPADRQQYLRQEVLRRAEATAASTSRSLALMYESEKVGVASSEELARQRGVLERTEKMVDKMDQDLKISQKHINSIKSVFGGLVNYFKSKPVETPPEQNGTLTSQPNNRLKEAISTSKEQEAKYQASHPNLRKLDDTDPVPRGAGSAMSTDAYPKNPHLRAYHQKIDSNLDELSMGLGRLKDIALGMQTEIEEQDDILDRLTTKVDKLDVNIKSTERKVRQL

SEQ ID NO: 5 - protein, 429 aa, Artificial Sequence
  Exemplary BoNT/A light chain variant with E148Y, K166F, N240S, S254A, and G305D substitutions
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEYLNLVIIGPSADIIQFECFSFGHEVLNLTRNGYGSTQYIRFSPDFTFGFEESLEVDTNPLLGAGKFATDPAVTLAHELIHAGHRLYGIAINPSRVFKVNTNAYYEMAGLEVSFEELRTFGGHDAKFIDSLQENEFRLYYYNKFKDIASTLNKAKSIVDTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLL

SEQ ID NO: 6 - protein, 429 aa, Artificial Sequence
  Exemplary BoNT/A light chain variant with E148Y, K166F, N240A, S254A and G305D substitutions
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEYLNLVIIGPSADIIQFECFSFGHEVLNLTRNGYGSTQYIRFSPDFTFGFEESLEVDTNPLLGAGKFATDPAVTLAHELIHAGHRLYGIAINPARVFKVNTNAYYEMAGLEVSFEELRTFGGHDAKFIDSLQENEFRLYYYNKFKDIASTLNKAKSIVDTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLL

SEQ ID NO: 7 - protein, 429 aa, Artificial Sequence
  Exemplary BoNT/A light chain variant with N26S, E148Y, K166F, N240A, S254A and G305D substitutions
MPFVNKQFNYKDPVNGVDIAYIKIPSAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEYLNLVIIGPSADIIQFECFSFGHEVLNLTRNGYGSTQYIRFSPDFTFGFEESLEVDTNPLLGAGKFATDPAVTLAHELIHAGHRLYGIAINPARVFKVNTNAYYEMAGLEVSFEELRTFGGHDAKFIDSLQENEFRLYYYNKFKDIASTLNKAKSIVDTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLL

SEQ ID NO: 8 - protein, 429 aa, Artificial Sequence
  Exemplary BoNT/A light chain variant with N26S, E55V, E148Y, K166F, N240A, S254A and G305D substitutions
MPFVNKQFNYKDPVNGVDIAYIKIPSAGQMQPVKAFKIHNKIWVIPERDTFTNPVEGDLNPPPEAKQVPVSYYDSTYLSTDNEKDNYLKGVTKLFERIYSTDLGRMLLTSIVRGIPFWGGSTIDTELKVIDTNCINVIQPDGSYRSEYLNLVIIGPSADIIQFECFSFGHEVLNLTRNGYGSTQYIRFSPDFTFGFEESLEVDTNPLLGAGKFATDPAVTLAHELIHAGHRLYGIAINPARVFKVNTNAYYEMAGLEVSFEELRTFGGHDAKFIDSLQENEFRLYYYNKFKDIASTLNKAKSIVDTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLL

SEQ ID NO: 9 - protein, 429 aa, Artificial Sequence
  Exemplary BoNT/A light chain variant with N26S, Q29R, E55V, E148Y, K166F, N240A, S254A and G305D substitutions
MPFVNKQFNYKDPVNGVDIAYIKIPSAGRMQPVKAFKIHNKIWVIPERDTFTNPVEGDLNPPPEAKQVPVSYYDSTYLSTDNEKDNYLKGVTKLFERIYSTDLGRMLLTSIVRGIPFWGGSTIDTELKVIDTNCINVIQPDGSYRSEYLNLVIIGPSADIIQFECFSFGHEVLNLTRNGYGSTQYIRFSPDFTFGFEESLEVDTNPLLGAGKFATDPAVTLAHELIHAGHRLYGIAINPARVFKVNTNAYYEMAGLEVSFEELRTFGGHDAKFIDSLQENEFRLYYYNKFKDIASTLNKAKSIVDTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLL

SEQ ID NO: 10 - protein, 429 aa, Artificial Sequence
  Exemplary BoNT/A light chain variant with N26S, Q29R, N53R, E55V, E148Y, K166F, N240A, S254A and G305D substitutions
MPFVNKQFNYKDPVNGVDIAYIKIPSAGRMQPVKAFKIHNKIWVIPERDTFTRPVEGDLNPPPEAKQVPVSYYDSTYLSTDNEKDNYLKGVTKLFERIYSTDLGRMLLTSIVRGIPFWGGSTIDTELKVIDTNCINVIQPDGSYRSEYLNLVIIGPSADIIQFECFSFGHEVLNLTRNGYGSTQYIRFSPDFTFGFEESLEVDTNPLLGAGKFATDPAVTLAHELIHAGHRLYGIAINPARVFKVNTNAYYEMAGLEVSFEELRTFGGHDAKFIDSLQENEFRLYYYNKFKDIASTLNKAKSIVDTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLL

SEQ ID NO: 11 - protein, 429 aa, Artificial Sequence
  Exemplary BoNT/A light chain variant with N26S, Q29R, N53R, E55V, E148Y, K166F, N240A, S254L and G305D substitutions
MPFVNKQFNYKDPVNGVDIAYIKIPSAGRMQPVKAFKIHNKIWVIPERDTFTRPVEGDLNPPPEAKQVPVSYYDSTYLSTDNEKDNYLKGVTKLFERIYSTDLGRMLLTSIVRGIPFWGGSTIDTELKVIDTNCINVIQPDGSYRSEYLNLVIIGPSADIIQFECFSFGHEVLNLTRNGYGSTQYIRFSPDFTFGFEESLEVDTNPLLGAGKFATDPAVTLAHELIHAGHRLYGIAINPARVFKVNTNAYYEMLGLEVSFEELRTFGGHDAKFIDSLQENEFRLYYYNKFKDIASTLNKAKSIVDTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLL

SEQ ID NO: 12 - protein, 429 aa, Artificial Sequence
  Exemplary BoNT/A light chain variant with N26S, Q29R, N53R, E55V, E148Y, K166F, N240A and S254L substitutions
MPFVNKQFNYKDPVNGVDIAYIKIPSAGRMQPVKAFKIHNKIWVIPERDTFTRPVEGDLNPPPEAKQVPVSYYDSTYLSTDNEKDNYLKGVTKLFERIYSTDLGRMLLTSIVRGIPFWGGSTIDTELKVIDTNCINVIQPDGSYRSEYLNLVIIGPSADIIQFECFSFGHEVLNLTRNGYGSTQYIRFSPDFTFGFEESLEVDTNPLLGAGKFATDPAVTLAHELIHAGHRLYGIAINPARVFKVNTNAYYEMLGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLL

SEQ ID NO: 13 - protein, 429 aa, Artificial Sequence
  Exemplary BoNT/A light chain variant with N26S, Q29R, N53H, E55V, E148Y, K166, N240A and S254L substitutions
MPFVNKQFNYKDPVNGVDIAYIKIPSAGRMQPVKAFKIHNKIWVIPERDTFTHPVEGDLNPPPEAKQVPVSYYDSTYLSTDNEKDNYLKGVTKLFERIYSTDLGRMLLTSIVRGIPFWGGSTIDTELKVIDTNCINVIQPDGSYRSEYLNLVIIGPSADIIQFECFSFGHEVLNLTRNGYGSTQYIRFSPDFTFGFEESLEVDTNPLLGAGKFATDPAVTLAHELIHAGHRLYGIAINPARVFKVNTNAYYEMLGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLL

SEQ ID NO: 14 - protein, 429 aa, Artificial Sequence
  Exemplary BoNT/A light chain variant with E148Y, K166F, E201D, D203V, S254A and G305D substitutions
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEYLNLVIIGPSADIIQFECFSFGHEVLNLTRNGYGSTQYIRFSPDFTFGFEESLDVVTNPLLGAGKFATDPAVTLAHELIHAGHRLYGIAINPNRVFKVNTNAYYEMAGLEVSFEELRTFGGHDAKFIDSLQENEFRLYYYNKFKDIASTLNKAKSIVDTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLL

SEQ ID NO: 15 - protein, 429 aa, Artificial Sequence
  Exemplary BoNT/A light chain variant with E148Y, K166F, S254A, G305D, K364R and Y387N substitutions
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEYLNLVIIGPSADIIQFECFSFGHEVLNLTRNGYGSTQYIRFSPDFTFGFEESLEVDTNPLLGAGKFATDPAVTLAHELIHAGHRLYGIAINPNRVFKVNTNAYYEMAGLEVSFEELRTFGGHDAKFIDSLQENEFRLYYYNKFKDIASTLNKAKSIVDTTASLQYMKNVFKEKYLLSEDTSGKFSVDKLKFDKLYKMLTEIYTEDNFVKFFKVLNRRTYLNFDKAVFKINIVPKVNYTINDGFNLRNTNLAANFNGQNTEINNMNFTKLKNFTGLFEFYKLL

SEQ ID NO: 16 - protein, 429 aa, Artificial Sequence
  Exemplary BoNT/A light chain variant with N26S, E148Y, Q162R, K166F, N240A, S254A and G305D substitutions
MPFVNKQFNYKDPVNGVDIAYIKIPSAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEYLNLVIIGPSADIIRFECFSFGHEVLNLTRNGYGSTQYIRFSPDFTFGFEESLEVDTNPLLGAGKFATDPAVTLAHELIHAGHRLYGIAINPARVFKVNTNAYYEMAGLEVSFEELRTFGGHDAKFIDSLQENEFRLYYYNKFKDIASTLNKAKSIVDTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLL

SEQ ID NO: 17 - protein, 429 aa, Artificial Sequence
  Exemplary BoNT/A light chain variant with N26S, E55N, E148Y, K166F, N240A, S254A and G305D substitutions
MPFVNKQFNYKDPVNGVDIAYIKIPSAGQMQPVKAFKIHNKIWVIPERDTFTNPNEGDLNPPPEAKQVPVSYYDSTYLSTDNEKDNYLKGVTKLFERIYSTDLGRMLLTSIVRGIPFWGGSTIDTELKVIDTNCINVIQPDGSYRSEYLNLVIIGPSADIIQFECFSFGHEVLNLTRNGYGSTQYIRFSPDFTFGFEESLEVDTNPLLGAGKFATDPAVTLAHELIHAGHRLYGIAINPARVFKVNTNAYYEMAGLEVSFEELRTFGGHDAKFIDSLQENEFRLYYYNKFKDIASTLNKAKSIVDTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLL

SEQ ID NO: 18 - protein, 429 aa, Artificial Sequence
  Exemplary BoNT/A light chain variant with N26S, E55I, E148Y, K166F, N240A, S254A and G305D substitutions
MPFVNKQFNYKDPVNGVDIAYIKIPSAGQMQPVKAFKIHNKIWVIPERDTFTNPIEGDLNPPPEAKQVPVSYYDSTYLSTDNEKDNYLKGVTKLFERIYSTDLGRMLLTSIVRGIPFWGGSTIDTELKVIDTNCINVIQPDGSYRSEYLNLVIIGPSADIIQFECFSFGHEVLNLTRNGYGSTQYIRFSPDFTFGFEESLEVDTNPLLGAGKFATDPAVTLAHELIHAGHRLYGIAINPARVFKVNTNAYYEMAGLEVSFEELRTFGGHDAKFIDSLQENEFRLYYYNKFKDIASTLNKAKSIVDTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLL

SEQ ID NO: 19 - protein, 429 aa, Artificial Sequence
  Exemplary BoNT/A light chain variant with N26S, Q29S, E55V, E148Y, K166F, N240A, S254A and G305D substitutions
MPFVNKQFNYKDPVNGVDIAYIKIPSAGSMQPVKAFKIHNKIWVIPERDTFTNPVEGDLNPPPEAKQVPVSYYDSTYLSTDNEKDNYLKGVTKLFERIYSTDLGRMLLTSIVRGIPFWGGSTIDTELKVIDTNCINVIQPDGSYRSEYLNLVIIGPSADIIQFECFSFGHEVLNLTRNGYGSTQYIRFSPDFTFGFEESLEVDTNPLLGAGKFATDPAVTLAHELIHAGHRLYGIAINPARVFKVNTNAYYEMAGLEVSFEELRTFGGHDAKFIDSLQENEFRLYYYNKFKDIASTLNKAKSIVDTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLL

SEQ ID NO: 20 - protein, 429 aa, Artificial Sequence
  Exemplary BoNT/A light chain variant with N26S, A27L, Q29R, N53R, E55V, E148Y, K166F, N240A, S254A and G305D substitutions
MPFVNKQFNYKDPVNGVDIAYIKIPSLGRMQPVKAFKIHNKIWVIPERDTFTRPVEGDLNPPPEAKQVPVSYYDSTYLSTDNEKDNYLKGVTKLFERIYSTDLGRMLLTSIVRGIPFWGGSTIDTELKVIDTNCINVIQPDGSYRSEYLNLVIIGPSADIIQFECFSFGHEVLNLTRNGYGSTQYIRFSPDFTFGFEESLEVDTNPLLGAGKFATDPAVTLAHELIHAGHRLYGIAINPARVFKVNTNAYYEMAGLEVSFEELRTFGGHDAKFIDSLQENEFRLYYYNKFKDIASTLNKAKSIVDTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLL

SEQ ID NO: 21 - protein, 429 aa, Artificial Sequence
  Exemplary BoNT/A light chain variant with N26S, A27R, Q29R, N53R, E55V, E148Y, K166F, N240A, S254A and G305D substitutions
MPFVNKQFNYKDPVNGVDIAYIKIPSRGRMQPVKAFKIHNKIWVIPERDTFTRPVEGDLNPPPEAKQVPVSYYDSTYLSTDNEKDNYLKGVTKLFERIYSTDLGRMLLTSIVRGIPFWGGSTIDTELKVIDTNCINVIQPDGSYRSEYLNLVIIGPSADIIQFECFSFGHEVLNLTRNGYGSTQYIRFSPDFTFGFEESLEVDTNPLLGAGKFATDPAVTLAHELIHAGHRLYGIAINPARVFKVNTNAYYEMAGLEVSFEELRTFGGHDAKFIDSLQENEFRLYYYNKFKDIASTLNKAKSIVDTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLL

SEQ ID NO: 22 - protein, 429 aa, Artificial Sequence
  Exemplary BoNT/A light chain variant with N26S, Q29R, N53R, E55V, E56I, E148Y, K166F, N240A, S254A and G305D substitutions
MPFVNKQFNYKDPVNGVDIAYIKIPSAGRMQPVKAFKIHNKIWVIPERDTFTRPVIGDLNPPPEAKQVPVSYYDSTYLSTDNEKDNYLKGVTKLFERIYSTDLGRMLLTSIVRGIPFWGGSTIDTELKVIDTNCINVIQPDGSYRSEYLNLVIIGPSADIIQFECFSFGHEVLNLTRNGYGSTQYIRFSPDFTFGFEESLEVDTNPLLGAGKFATDPAVTLAHELIHAGHRLYGIAINPARVFKVNTNAYYEMAGLEVSFEELRTFGGHDAKFIDSLQENEFRLYYYNKFKDIASTLNKAKSIVDTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLL

SEQ ID NO: 23 - protein, 429 aa, Artificial Sequence
  Exemplary BoNT/A light chain variant with N26S, Q29R, N53R, E55V, E148Y, K166F, N240A, S254M and G305D substitutions
MPFVNKQFNYKDPVNGVDIAYIKIPSAGRMQPVKAFKIHNKIWVIPERDTFTRPVEGDLNPPPEAKQVPVSYYDSTYLSTDNEKDNYLKGVTKLFERIYSTDLGRMLLTSIVRGIPFWGGSTIDTELKVIDTNCINVIQPDGSYRSEYLNLVIIGPSADIIQFECFSFGHEVLNLTRNGYGSTQYIRFSPDFTFGFEESLEVDTNPLLGAGKFATDPAVTLAHELIHAGHRLYGIAINPARVFKVNTNAYYEMMGLEVSFEELRTFGGHDAKFIDSLQENEFRLYYYNKFKDIASTLNKAKSIVDTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLL

SEQ ID NO: 24 - protein, 585 aa, Artificial Sequence
  SNAP23 substrate
MHHHHHHENLYFQGIFRAPMASKGEELFTGVVPILVELDGDVNGHKFSVSGEGEGDATYGKLTLKFICTTGKLPVPWPTLVTTLCYGVQCFSRYPDHMKRHDFFKSAMPEGYVQERTIFFKDDGNYKTRAEVKFEGDTLVNRIELKGIDFKEDGNILGHKLEYNYNSHNVYIMADKQKNGIKVNFKTRHNIEDGSVQLADHYQQNTPIGDGPVLLPDNHYLSTQSALSKDPNEKRDHMVLLEFVTAAGITHGMDELYNGGAGSGAGGGGYIKRITNDAREDEMEENLTQVGSILGNLKDMALNIGNEIDAQNPQIKRITDKADTNRDRIDIANARAKKLIDSGGGSSASKGEELFTGVVPILVELDGDVNGHKFSVSGEGGGDATYGKLTLKFICTTGKLPVPWPTLVTTLSHGVQCFSRYPDHMKRHDFFKSAMPEGYVQERTIFFKDDGNYKTRAEVKFEGDTLVNRIELKGIDFKEDGNILGHKLEYNYNSHNVYIMADKQKNGIKANFKIRHNIEDGSVQLADHYQQNTPIGDGPVLLPDNHYLSTQSALSKDPNEKRDHMVLLEFVTAAGITHGMDELYK

SEQ ID NO: 25 - protein, 585 aa, Artificial Sequence
  SNAP25 substrate
MHHHHHHENLYFQGIFRAPMASKGEELFTGVVPILVELDGDVNGHKFSVSGEGEGDATYGKLTLKFICTTGKLPVPWPTLVTTLCYGVQCFSRYPDHMKRHDFFKSAMPEGYVQERTIFFKDDGNYKTRAEVKFEGDTLVNRIELKGIDFKEDGNILGHKLEYNYNSHNVYIMADKQKNGIKVNFKTRHNIEDGSVQLADHYQQNTPIGDGPVLLPDNHYLSTQSALSKDPNEKRDHMVLLEFVTAAGITHGMDELYNGGAGSGAGGGGIRRVTNDARENEMDENLEQVSGIIGNLRHMALDMGNEIDTQNRQIDRIMEKADSNKTRIDEANQRATKMLGSGGGGGTASKGEELFTGVVPILVELDGDVNGHKFSVSGEGEGDATYGKLTLKFICTTGKLPVPWPTLVTTLSHGVQCFSRYPDHMKRHDFFKSAMPEGYVQERTIFFKDDGNYKTRAEVKFEGDTLVNRIELKGIDFKEDGNILGHKLEYNYNSHNVYIMADKQKNGIKANFKIRHNIEDGSVQLADHYQQNTPIGDGPVLLPDNHYLSTQSALSKDPNEKRDHMVLLEFVTAAGITHGMDELYK

SEQ ID NO: 26 - protein, 584 aa, Artificial Sequence
  SNAP29 substrate
MHHHHHHENLYFQGIFRAPMASKGEELFTGVVPILVELDGDVNGHKFSVSGEGEGDATYGKLTLKFICTTGKLPVPWPTLVTTLCYGVQCFSRYPDHMKRHDFFKSAMPEGYVQERTIFFKDDGNYKTRAEVKFEGDTLVNRIELKGIDFKEDGNILGHKLEYNYNSHNVYIMADKQKNGIKVNFKTRHNIEDGSVQLADHYQQNTPIGDGPVLLPDNHYLSTQSALSKDPNEKRDHMVLLEFVTAAGITHGMDELYNGGAGSGAGGSTDAYPKNPHLRAYHQKIDSNLDELSMGLGRLKDIALGMQTEIEEQDDILDRLTTKVDKLDVNIKSTERKVRQLGGGSSVSKGEELFTGVVPILVELDGDVNGHKFSVSGEGGGDATYGKLTLKFICTTGKLPVPWPTLVTTLSHGVQCFSRYPDHMKRHDFFKSAMPEGYVQERTIFFKDDGNYKTRAEVKFEGDTLVNRIELKGIDFKEDGNILGHKLEYNYNSHNVYIMADKQKNGIKANFKIRHNIEDGSVQLADHYQQNTPIGDGPVLLPDNHYLSTQSALSKDPNEKRDHMVLLEFVTAAGITHGMDELYK

SEQ ID NO: 27 - protein, 429 aa, Artificial Sequence
  BoNT/A light chain quadruple mutant (qmLC/A) with E148Y, K166F, S254A, and G305D substitutions
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEYLNLVIIGPSADIIQFECFSFGHEVLNLTRNGYGSTQYIRFSPDFTFGFEESLEVDTNPLLGAGKFATDPAVTLAHELIHAGHRLYGIAINPNRVFKVNTNAYYEMAGLEVSFEELRTFGGHDAKFIDSLQENEFRLYYYNKFKDIASTLNKAKSIVDTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLL

SEQ ID NO: 28 - protein, 411 aa, Artificial Sequence
  BoNT/E light chain with three (3) amino acid differences from the amino acid residues 1 - 411 of UniProt Q00496
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHGFGSIAIVTFSPEYSFRFNDNSMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRF

SEQ ID NO: 29 - protein, 584 aa, Artificial Sequence
  SNAP25/29 chimeric substrate
MHHHHHHENLYFQGIFRAPMASKGEELFTGVVPILVELDGDVNGHKFSVSGEGEGDATYGKLTLKFICTTGKLPVPWPTLVTTLCYGVQCFSRYPDHMKRHDFFKSAMPEGYVQERTIFFKDDGNYKTRAEVKFEGDTLVNRIELKGIDFKEDGNILGHKLEYNYNSHNVYIMADKQKNGIKVNFKTRHNIEDGSVQLADHYQQNTPIGDGPVLLPDNHYLSTQSALSKDPNEKRDHMVLLEFVTAAGITHGMDELYNGGAGSGAGGSTDAYPKNPHLRAYHQKIDSNLDELSMGLGRLKDIALGMGNEIDTQNRQIDRIMEKADKLDVNIKSTERKVRQLGGGSSVSKGEELFTGVVPILVELDGDVNGHKFSVSGEGGGDATYGKLTLKFICTTGKLPVPWPTLVTTLSHGVQCFSRYPDHMKRHDFFKSAMPEGYVQERTIFFKDDGNYKTRAEVKFEGDTLVNRIELKGIDFKEDGNILGHKLEYNYNSHNVYIMADKQKNGIKANFKIRHNIEDGSVQLADHYQQNTPIGDGPVLLPDNHYLSTQSALSKDPNEKRDHMVLLEFVTAAGITHGMDELYK

SEQ ID NO: 30 - protein, 1296 aa, Artificial Sequence
  Full-length wild-type BoNT/A (amino acid residues 1 - 1296 of UniProt P0DPI1)
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 31 - protein, 411 aa, Artificial Sequence
  Amino acid residues 1 - 411 of wild-type BoNT/E light chain from UniProt Q00496
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHRFGSIAIVTFSPEYSFRFNDNCMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLRTKFQVKCRQTYIGQYKYFKLSNLLNDSIYNISEGYNINNLKVNFRGQNANLNPRIITPITGRGLVKKIIRF

SEQ ID NO: 32 - protein, 25 aa, Artificial Sequence
  Activation loop of BoNT/A protease
CVRGIITSKTKSLDKGYNKALNDLC